(19) United States
(12) Patent Application Publication
(10) Pub. No.: US 2020/0370063 A1
(43) Pub. Date: Nov. 26, 2020

(54) GENETICALLY ENGINEERED LAND PLANTS THAT EXPRESS LCID/E PROTEIN AND OPTIONALLY A CCP1 MITOCHONDRIAL TRANSPORTER PROTEIN AND/OR PYRUVATE CARBOXYLASE

(71) Applicant: YIELD10 BIOSCIENCE, INC., Woburn, MA (US)

(72) Inventors: Frank Anthony SKRALY, Woburn, MA (US); Kristi D. SNELL, Woburn, MA (US)

(21) Appl. No.: 16/766,789

(22) PCT Filed: Nov. 26, 2018
(86) PCT No.: PCT/US2018/062468

§ 371 Date: May 26, 2020

Related U.S. Application Data
(60) Provisional application No. 62/590,793, filed on Nov. 27, 2017.
(60) Provisional application No. 62/690,148, filed on Jun. 26, 2018.

(51) Int. Cl. C12N 15/82; C07K 14/415; C07K 14/405

(52) U.S. Cl. CPC C12N 15/8261; C07K 14/405; C07K 14/415

(57) ABSTRACT

A genetically engineered land plant that expresses an LCID/E protein is provided. The plant comprises a modified gene for the LCID/E protein. The LCID/E protein comprises (i) LCD of Chlamydomonas reinhardtii of SEQ ID NO: 4, (ii) LCIE of Chlamydomonas reinhardtii of SEQ ID NO: 5, or (iii) an algal or plant ortholog of LCID/E. The LCID/E protein is localized to chloroplasts of the plant based on a plastidial targeting signal. The modified gene for the LCID/E protein comprises (i) a promoter and (ii) a nucleic acid sequence encoding the LCID/E protein. The promoter is non-cognate with respect to the nucleic acid sequence encoding the LCID/E protein. The modified gene for the LCID/E protein is configured such that transcription of the nucleic acid sequence is initiated from the promoter and results in expression of the LCID/E protein. Optionally, the plant also expresses a CCP1 mitochondrial transporter protein and/or pyruvate carboxylase.

### FIELD OF THE INVENTION

The present invention relates generally to genetically engineered land plants that express an LCID/E protein, and more particularly, to such genetically engineered land plants comprising a modified gene for the LCID/E protein, and, optionally, that express a CCP1 mitochondrial transporter protein and/or pyruvate carboxylase.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population. Increased demand for improved diet, concomitant land use changes for new living space and infrastructure, alternative uses for crops and changing weather patterns will add to the challenge.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. Crop productivity is also limited by the availability of water. Achieving step changes in crop yield requires new approaches.

One potential approach for achieving step changes in crop yield involves metabolic engineering of crop plants to express carbon-concentrating mechanisms of cyanobacteria or eukaryotic algae. Cyanobacteria and eukaryotic algae have evolved carbon-concentrating mechanisms to increase intracellular concentrations of dissolved inorganic carbon, particularly to increase concentrations of CO₂ at the active site of ribulose-1,5-bisphosphate carboxylase/oxygenase (also termed RuBisCO). A family of low carbon inducible proteins has been identified in the algal species Chlamydomonas reinhardtii, with the family including CCP1, CCP2, LCIA, LCIB, LCIC, LCD, and LCIE, among other proteins.

It has recently been shown by Schnell et al., WO 2015/103074, that Camelina plants transformed to express CCP1 of the eukaryotic algal species Chlamydomonas reinhardtii have reduced transpiration rates, increased CO₂ assimilation rates and higher yield than control plants which do not express the CCP1 gene. More recently, Atkinson et al., (2015) Plant Biotechnol. J., doi: 10.1111/pbi.12497, discloses that CCP1 and its homolog CCP2, which were previously characterized as Ci transporters, previously reported to be in the chloroplast envelope, localized to mitochondria in both Chlamydomonas reinhardtii, as expressed naturally, and tobacco, when expressed heterologously, suggesting that the model for the carbon-concentrating mechanism of eukaryotic algae needs to be expanded to include a role for mitochondria. Atkinson et al. (2015) disclosed that expression of individual Ci (bicarbonate) transporters did not enhance growth of the plant Arabidopsis.

In co-pending Patent Application PCT/US2017/016421, to Yield10 Bioscience, a number of orthologs of CCP1 from algal species that share common protein sequence domains including mitochondrial membrane domains and transporter protein domains were shown to increase seed yield and reduce seed size when expressed constitutively in Camelina plants. Schnell et al., WO 2015/103074, also reported a decrease in seed size in higher yielding Camelina lines expressing CCP1.

In U.S. Provisional Application 62/462,074, to Yield10 Bioscience, CCP1 and its orthologs from other algae are referred to as mitochondrial transporter proteins. The inventors tested the impact of expressing CCP1 or its algal orthologs using seed-specific promoters with the unexpected outcome that both seed yield and seed size increased. These inventors also recognized the benefits of combining constitutive expression and seed specific expression of CCP1 or any of its orthologs in the same plant.

In U.S. Provisional Application 62/520,785, to Yield10 Bioscience, genetically engineered land plants that express a plant CCP1-like mitochondrial transporter protein are disclosed. The genetically engineered land plants include a modified gene for the plant CCP1-like mitochondrial transporter protein. The modified gene includes a promoter and a nucleic acid sequence encoding the plant CCP1-like mitochondrial transporter protein. The promoter is non-cognate with respect to the nucleic acid sequence.

Of the other low carbon inducible proteins of Chlamydomonas reinhardtii, the functions of LCID and LCIE have not yet been determined. Sequence alignments of LCIA, LCIB, LCIC, LCD, and LCIE indicate that LCIA is distinct from LCIB, LCIC, LCD, and LCIE, in that LCIA contains additional amino acid residues at its N-terminus, and lacks amino acids at its C-terminus, in comparison to LCIB, LCIC, LCD, and LCIE. Alignments of LCIB, LCIC, LCD, and LCIE indicate that LCID and LCIE differ from LCIB and LCIC with respect to corresponding N-terminal domains of about 100 amino acids. A recent review of Wang et al., 2015, Plant J. 82:429-448, indicates that LCIB and LCIC are located in chloroplast stroma, whereas the locations of LCID and LCIE are unknown, and that neither LCID nor LCIE has been confirmed to function in Ci uptake. A reference of Wang et al., 2011, Photosynth. Res. 109:115-122, indicates that LCIB and LCIC are among the most abundant transcripts upon induction due to carbon limitation, whereas LCID and especially LCIE have much lower transcript abundances under conditions tested. Spalding, WO2013/006361, reports overexpression of LCIA and LCIB in algae and show that algae had higher biomass production at elevated carbon dioxide levels. Spalding mentions LCID and LCIE, but provides no data regarding expression of these proteins. Nölke, W02016/087314, express LCIA and LCIB, among other proteins, in tobacco. Nölke also mentions LCID and LCIE, but also provides no data regarding expression of these proteins. Accordingly, it is not apparent whether or to what extent LCID and/or LCIE may play roles in carbon-concentrating mechanisms to increase intracellular concentrations of dissolved inorganic carbon.

Another potential approach for achieving step changes in crop yield involves transforming plants with transgenic polynucleotides encoding one or more metabolic enzymes. For example, Malik et al., WO 2016/164810, reports methods of using novel metabolic pathways having enzymes catalyzing carboxylation reactions and/or enzymes using NADPH or NADH as a cofactor to enhance the yield of desirable crop traits. In one embodiment, the transgenic plant comprises one or more transgenes encoding two, three, four, five, six, seven, eight or more enzymes selected from the group: an oxygen tolerant pyruvate oxidoreductase, pyruvate carboxylase (also termed PYC), malate synthase, malate dehydrogenase, malate thiokinase, malyl-CoA lyase, and isocitrate lyase, wherein the transgenic plant is selected on the basis of having a higher yield in comparison with a corresponding plant that is not expressing the heterologous enzyme(s).

Regarding pyruvate carboxylase, Hanke et al., U.S. Pat. No. 6,965,021 discloses that in bacteria such as Corynebacterium glutamicum, pyruvate carboxylase is utilized during carbohydrate metabolism to form oxaloacetate, which is in turn used in the biosynthesis of amino acids, particularly L-lysine and L-glutamate. Hanke et al. also discloses that in response to a cell's metabolic needs and internal environment, the activity of pyruvate carboxylase is subject to both positive and negative feedback mechanisms, where the enzyme is activated by acetyl-CoA, and inhibited by aspartic acid. Hanke et al. discloses a nucleic acid molecule comprising a nucleotide sequence that codes for a pyruvate carboxylase that contains at least one mutation that desensitizes the pyruvate carboxylase to feedback inhibition by aspartic acid.

Unfortunately, “transgenic plants,” “GMO crops,” and/or “biotech traits” are not widely accepted in some regions and countries and are subject to regulatory approval processes that are very time consuming and prohibitively expensive. The current regulatory framework for transgenic plants results in significant costs (˜$136 million per trait; McDougall, P. 2011, “The cost and time involved in the discovery, development, and authorization of a new plant biotechnology derived trait.” Crop Life International) and lengthy product development timelines that limit the number of technologies that are brought to market. This has severely impaired private investment and the adoption of innovation in this crucial sector. Recent advances in genome editing technologies provide an opportunity to precisely remove genes or edit control sequences to significantly improve plant productivity (Belhaj, K. 2013, Plant Methods, 9, 39; Khandagale & Nadal, 2016, Plant Biotechnol Rep, 10, 327) and open the way to produce plants that may benefit from an expedited regulatory path, or possibly unregulated status.

Given the costs and challenges associated with obtaining regulatory approval and societal acceptance of transgenic crops there is a need to identify, where possible, plant transporter proteins, ideally derived from crops or other land plants, that can be genetically engineered to enable enhanced carbon capture systems to improve crop yield and/or seed yield, particularly without relying on genes, control sequences, or proteins derived from non-land plants to the extent possible.

### BRIEF SUMMARY OF THE INVENTION

A genetically engineered land plant that expresses an LCID/E protein is provided. The plant comprises a modified gene for the LCID/E protein. The LCID/E protein comprises (i) LCID of Chlamydomonas reinhardtii of SEQ ID NO: 4, (ii) LCIE of Chlamydomonas reinhardtii of SEQ ID NO: 5, or (iii) an algal or plant ortholog of LCID/E. The LCID/E protein is localized to chloroplasts of the genetically engineered land plant based on a plastidial targeting signal. The modified gene for the LCID/E protein comprises (i) a promoter and (ii) a nucleic acid sequence encoding the LCID/E protein. The promoter is non-cognate with respect to the nucleic acid sequence encoding the LCID/E protein. The modified gene for the LCID/E protein is configured such that transcription of the nucleic acid sequence encoding the LCID/E protein is initiated from the promoter and results in expression of the LCID/E protein.

In some embodiments the genetically engineered land plant further expresses a CCP1 mitochondrial transporter protein. In accordance with these embodiments, the genetically engineered land plant comprises a modified gene for the CCP1 mitochondrial transporter protein. The CCP1 mitochondrial transporter protein comprises: (i) CCP1 of Chlamydomonas reinhardtii of SEQ ID NO: 9 or (ii) an ortholog of CCP1. The CCP1 mitochondrial transporter protein is localized to mitochondria of the genetically engineered land plant based on a mitochondrial targeting signal. The modified gene for the CCP1 mitochondrial transporter protein comprises (i) another promoter and (ii) a nucleic acid sequence encoding the CCP1 mitochondrial transporter protein. The other promoter is non-cognate with respect to the nucleic acid sequence. The modified gene for the CCP1 mitochondrial transporter protein is configured such that transcription of the nucleic acid sequence encoding the CCP1 mitochondrial transporter protein is initiated from the other promoter and results in expression of the CCP1 mitochondrial transporter protein.

Also in some embodiments, the genetically engineered land plant further expresses a pyruvate carboxylase. In accordance with these embodiments, the genetically engineered land plant comprises a modified gene for the pyruvate carboxylase. The modified gene for the pyruvate carboxylase comprises (i) a further promoter and (ii) a nucleic acid sequence encoding the pyruvate carboxylase. The further promoter is non-cognate with respect to the nucleic acid sequence encoding the pyruvate carboxylase. The modified gene for the pyruvate carboxylase is configured such that transcription of the nucleic acid sequence encoding the pyruvate carboxylase is initiated from the further promoter and results in expression of the pyruvate carboxylase.

Exemplary embodiments include the following.

Embodiment 1: A genetically engineered land plant that expresses an LCID/E protein, the genetically engineered land plant comprising a modified gene for the LCID/E protein, wherein:

the LCID/E protein comprises (i) LCD of Chlamydomonas reinhardtii of SEQ ID NO: 4, (ii) LCIE of Chlamydomonas reinhardtii of SEQ ID NO: 5, or (iii) an algal or plant ortholog of LCID/E;

the LCID/E protein is localized to chloroplasts of the genetically engineered land plant based on a plastidial targeting signal;

the modified gene for the LCID/E protein comprises (i) a promoter and (ii) a nucleic acid sequence encoding the LCID/E protein;

the promoter is non-cognate with respect to the nucleic acid sequence encoding the LCID/E protein; and

the modified gene for the LCID/E protein is configured such that transcription of the nucleic acid sequence encoding the LCID/E protein is initiated from the promoter and results in expression of the LCID/E protein.

Embodiment 2: The genetically engineered land plant of Embodiment 1, wherein the LCID/E protein comprises the algal or plant ortholog of LCID/E based on comprising: (i) (a) a glutamate residue at position 161, (b) a cysteine residue at position 189, (c) a cysteine residue at position 241, (d) an aspartate residue at position 310, and (e) a glutamate residue at position 312, with numbering of positions relative to LCID of Chlamydomonas reinhardtii of SEQ ID NO: 4, and (ii) an overall identity of at least 15%.

Embodiment 3: The genetically engineered land plant of Embodiment 1 or 2, wherein the LCID/E protein comprises the algal or plant ortholog of LCID/E based on comprising: (i) (a) an asparagine residue at position 233, (b) a lysine residue at position 322, and (c) a glutamine residue at position 405, with numbering of positions relative to LCID of Chlamydomonas reinhardtii of SEQ ID NO: 4, and (ii) an overall identity of at least 15%.

Embodiment 4: The genetically engineered land plant of any one of Embodiments 1-3, wherein the LCID/E protein comprises the algal or plant ortholog of LCID/E based on comprising: (i) one or more LCID/E signature sequences of (a) FSFPHI (SEQ ID NO: 13) at position 213-218, (b) ACGAL (SEQ ID NO: 14) at position 240-244, (c) ADYAV (SEQ ID NO: 15) at position 324-328, or (d) TGVQIHNW (SEQ ID NO: 16) at position 330-337, with numbering of positions relative to LCID of Chlamydomonas reinhardtii of SEQ ID NO: 4, and (ii) an overall identity of at least 60%.

Embodiment 5: The genetically engineered land plant of any one of Embodiments 1-4, wherein the LCID/E protein comprises at least one of (a) an LCID/E protein of Zea nicaraguensis, (b) an LCID/E protein of Cosmos bipinnatus, or (c) an LCID/E protein of Nymphoides peltata.

Embodiment 6: The genetically engineered land plant of Embodiment 5, wherein the LCID/E protein comprises an LCID/E protein of Zea nicaraguensis.

Embodiment 7: The genetically engineered land plant of any one of Embodiments 1-4, wherein the LCID/E protein comprises at least one of (a) an LCID/E protein of Zea nicaraguensis of SEQ ID NO: 6, (b) an LCID/E protein of Cosmos bipinnatus of SEQ ID NO: 7, or (c) an LCID/E protein of Nymphoides peltata of SEQ ID NO: 8.

Embodiment 8: The genetically engineered land plant of Embodiment 7, wherein the LCID/E protein comprises an LCID/E protein of Zea nicaraguensis of SEQ ID NO: 6.

Embodiment 9: The genetically engineered land plant of any one of Embodiments 1-8, wherein the LCID/E protein consists essentially of an amino acid sequence that is identical to that of a wild-type LCID/E protein.

Embodiment 10: The genetically engineered land plant of any one of Embodiments 1-9, wherein the LCID/E protein is heterologous with respect to the genetically engineered land plant.

Embodiment 11: The genetically engineered land plant of any one of Embodiments 1-9, wherein the LCID/E protein is homologous with respect to the genetically engineered land plant.

Embodiment 12: The genetically engineered land plant of any one of Embodiments 1-11, wherein the promoter is a constitutive promoter.

Embodiment 13: The genetically engineered land plant of any one of Embodiments 1-12, wherein the promoter is a seed-specific promoter.

Embodiment 14: The genetically engineered land plant of any one of Embodiments 1-13, wherein the modified gene for the LCID/E protein is integrated into genomic DNA of the genetically engineered land plant.

Embodiment 15: The genetically engineered land plant of any one of Embodiments 1-14, wherein the modified gene for the LCID/E protein is stably expressed in the genetically engineered land plant.

Embodiment 16: The genetically engineered land plant of any of Embodiments 1-15, wherein the genetically engineered land plant has a CO₂ assimilation rate that is at least 5% higher, at least 10% higher, at least 20% higher, or at least 40% higher, than for a corresponding reference land plant that does not comprise the modified gene for the LCID/E protein.

Embodiment 17: The genetically engineered land plant of any of Embodiments 1-16, wherein the genetically engineered land plant has a transpiration rate that is at least 5% lower, at least 10% lower, at least 20% lower, or at least 40% lower, than for a corresponding reference land plant that does not comprise the modified gene for the LCID/E protein.

Embodiment 18: The genetically engineered land plant of any of Embodiments 1-17, wherein the genetically engineered land plant has a seed yield that is at least 5% higher, at least 10% higher, at least 20% higher, at least 40% higher, at least 60% higher, or at least 80% higher, than for a corresponding reference land plant that does not comprise the modified gene for the LCID/E protein.

Embodiment 19: The genetically engineered land plant of any of Embodiments 1-18, wherein the genetically engineered land plant is a C3 plant.

Embodiment 20: The genetically engineered land plant of any of Embodiments 1-19, wherein the genetically engineered land plant is a C4 plant.

Embodiment 21: The genetically engineered land plant of any of Embodiments 1-20, wherein the genetically engineered land plant is a food crop plant selected from the group consisting of maize, wheat, oat, barley, soybean, millet, sorghum, potato, pulse, bean, tomato, and rice.

Embodiment 22: The genetically engineered land plant of Embodiment 21, wherein the genetically engineered land plant is maize.

Embodiment 23: The genetically engineered land plant of any of Embodiments 1-20, wherein the genetically engineered land plant is a forage crop plant selected from the group consisting of silage corn, hay, and alfalfa.

Embodiment 24: The genetically engineered land plant of Embodiment 23, wherein the genetically engineered land plant is silage corn.

Embodiment 25: The genetically engineered land plant of any of Embodiments 1-20, wherein the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

Embodiment 26: The genetically engineered land plant of any one of Embodiments 1-25, wherein the genetically engineered land plant further expresses a CCP1 mitochondrial transporter protein, the genetically engineered land plant comprising a modified gene for the CCP1 mitochondrial transporter protein, further wherein:

the CCP1 mitochondrial transporter protein comprises: (i) CCP1 of Chlamydomonas reinhardtii of SEQ ID NO: 9 or (ii) an ortholog of CCP1;

the CCP1 mitochondrial transporter protein is localized to mitochondria of the genetically engineered land plant based on a mitochondrial targeting signal;

the modified gene for the CCP1 mitochondrial transporter protein comprises (i) another promoter and (ii) a nucleic acid sequence encoding the CCP1 mitochondrial transporter protein;

the other promoter is non-cognate with respect to the nucleic acid sequence encoding the CCP1 mitochondrial transporter protein; and

the modified gene for the CCP1 mitochondrial transporter protein is configured such that transcription of the nucleic acid sequence encoding the CCP1 mitochondrial transporter protein is initiated from the other promoter and results in expression of the CCP1 mitochondrial transporter protein.

Embodiment 27: The genetically engineered land plant of Embodiment 26, wherein the ortholog of CCP1 comprises an algal CCP1 ortholog.

Embodiment 28: The genetically engineered land plant of Embodiment 27, wherein the algal CCP1 ortholog comprises a CCP1 ortholog of Gonium pectorals of SEQ ID NO: 44 or SEQ ID NO: 45, Volvox carteri f. nagariensis of SEQ ID NO: 46, Volvox carteri of SEQ ID NO: 47, Ettlia oleoabundans of SEQ ID NO: 48, Chlorella sorokiniana of SEQ ID NO: 49, Chlorella variabilis of SEQ ID NO: 50, SEQ ID NO: 51, SEQ ID NO: 52, or SEQ ID NO: 54, or Chondrus crispus of SEQ ID NO: 53, SEQ ID NO: 55, or SEQ ID NO: 56.

Embodiment 29: The genetically engineered land plant of Embodiment 26, wherein the ortholog of CCP1 comprises a plant CCP1 ortholog.

Embodiment 30: The genetically engineered land plant of Embodiment 29, wherein the plant CCP1 ortholog comprises a CCP1 ortholog of Erigeron breviscapus of SEQ ID NO: 57, Zea nicaraguensis of SEQ ID NO: 58, Poa pratensis of SEQ ID NO: 59, Cosmos bipinnatus of SEQ ID NO: 60, Glycine max of SEQ ID NO: 61, Zea mays of SEQ ID NO: 62, Oryza sativa of SEQ ID NO: 63, Triticum aestivum of SEQ ID NO: 64, Sorghum bicolor of SEQ ID NO: 65, or Solanum tuberosum of SEQ ID NO: 66.

Embodiment 31: The genetically engineered land plant of any one of Embodiments 1-30, wherein the genetically engineered land plant further expresses a pyruvate carboxylase, the genetically engineered land plant comprising a modified gene for the pyruvate carboxylase, further wherein:

the modified gene for the pyruvate carboxylase comprises (i) a further promoter and (ii) a nucleic acid sequence encoding the pyruvate carboxylase;

the further promoter is non-cognate with respect to the nucleic acid sequence encoding the pyruvate carboxylase; and

the modified gene for the pyruvate carboxylase is configured such that transcription of the nucleic acid sequence encoding the pyruvate carboxylase is initiated from the further promoter and results in expression of the pyruvate carboxylase.

Embodiment 32: The genetically engineered land plant of Embodiment 31, wherein the pyruvate carboxylase comprises a bacterial pyruvate carboxylase.

Embodiment 33: The genetically engineered land plant of Embodiment 32, wherein the bacterial pyruvate carboxylase comprises a pyruvate carboxylase of Corynebacterium glutamicum of SEQ ID NO. 78 or Bacillus subtilus of SEQ ID NO: 80.

Embodiment 34: The genetically engineered land plant of Embodiment 31, wherein the pyruvate carboxylase comprises an algal pyruvate carboxylase.

Embodiment 35: The genetically engineered land plant of Embodiment 34, wherein the algal pyruvate carboxylase comprises a pyruvate carboxylase of Chlamydomonas reinhardtii of SEQ ID NO: 72, Chlorella variabilis of SEQ ID NO: 74, or Chlorella sorokiniana of SEQ ID NO: 76 or SEQ ID NO: 77.

Embodiment 36: The genetically engineered land plant of Embodiment 31, wherein the pyruvate carboxylase comprises a pyruvate carboxylase that is desensitized to feedback inhibition from aspartic acid.

Embodiment 37: The genetically engineered land plant of Embodiment 36, wherein the pyruvate carboxylase that is desensitized to feedback inhibition from aspartic acid is desensitized based on comprising one or more of: (a) an aspartate residue at position 153, (b) a serine residue at position 182, (c) a serine residue at position 206, (d) an arginine residue at position 227, (e) a glycine residue at position 455, or (f) a glutamate residue at position 1120, with numbering of positions relative to pyruvate carboxylase of Corynebacterium glutamicum of SEQ ID NO. 78.

Embodiment 38: The genetically engineered land plant of Embodiment 36, wherein the pyruvate carboxylase that is desensitized to feedback inhibition from aspartic acid comprises a mutated pyruvate carboxylase of Corynebacterium glutamicum of SEQ ID NO. 79.

Embodiment 39: The genetically engineered land plant of any one of Embodiments 31-38, wherein the pyruvate carboxylase is heterologous with respect to the genetically engineered land plant.

Embodiment 40: The genetically engineered land plant of any one of Embodiments 31-39, wherein the further promoter is a constitutive promoter.

Embodiment 41: The genetically engineered land plant of any one of Embodiments 31-39, wherein the further promoter is a leaf-specific promoter.

Embodiment 42: The genetically engineered land plant of any one of Embodiments 31-39, wherein the further promoter is a seed-specific promoter.

Embodiment 43: The genetically engineered land plant of Embodiment 42, wherein the pyruvate carboxylase is expressed in cytosol and/or targeted to plastid.

### BRIEF DESCRIPTION OF THE DRAWINGS

FIG. 1 shows the genomic arrangement of (A) CCP1/LCIE and CCP2/LCID in Chlamydomonas reinhardtii and (B) CCP1/LCIE homologs in Gonium pectorals.

FIG. 2A-B shows a multiple sequence alignment of the Chlamydomonas reinhardtii LCIB (SEQ ID NO: 2), LCIC (SEQ ID NO: 3), LCD (SEQ ID NO: 4), and LCIE (SEQ ID NO: 5) proteins according to CLUSTAL 0(1.2.4).

FIG. 3A-B shows plasmid maps of plant transformation vectors pYTEN1 (SEQ ID NO: 67) and pYTEN2 (SEQ ID NO: 68). Plasmid pYTEN1 contains a constitutive expression cassette, driven by the CaMV35S promoter, for expression of the LCIE gene from Chlamydomonas reinhardtii. The LCIE gene has been codon optimized for expression in Arabidopsis. Plasmid pYTEN2 contains a seed-specific expression cassette, driven by the promoter from the soya bean oleosin isoform A gene, for expression of the LCIE gene from Chlamydomonas reinhardtii. The LCIE gene has been codon optimized for expression in Arabidopsis. For both plasmids, an expression cassette for the bar gene, driven by the CaMV35S promoter, imparts transgenic plants resistance to the herbicide bialophos.

FIG. 4A-B shows plasmid maps of plant transformation vectors pYTEN3 (SEQ ID NO: 69) and pYTEN4 (SEQ ID NO: 70). Plasmid pYTEN3 contains constitutive expression cassettes, driven by the CaMV35S promoter, for expression of the CCP1 and LCIE genes from Chlamydomonas reinhardtii. The LCIE gene has been codon optimized for expression in Arabidopsis. Plasmid pYTEN4 contains seed-specific expression cassettes, driven by the promoter from the soybean oleosin isoform A gene, for expression of the CCP1 and LCIE genes from Chlamydomonas reinhardtii. The LCIE gene has been codon optimized for expression in Arabidopsis. For both plasmids, an expression cassette for the bar gene, driven by the CaMV35S promoter, imparts transgenic plants resistance to the herbicide bialaphos.

FIG. 5A-D shows a multiple sequence alignment of the Chlamydomonas reinhardtii LCIA (SEQ ID NO: 1), LCIB (SEQ ID NO: 2), LCIC (SEQ ID NO: 3), LCID (SEQ ID NO: 4), and LCIE (SEQ ID NO: 5) proteins and LCID/E orthologs of Zea nicaraguensis (SEQ ID NO: 6), Cosmos bipinnatus (SEQ ID NO: 7), and Nymphoides peltata (SEQ ID NO: 8) according to CLUSTAL 0(1.2.4).

FIG. 6A-C shows a multiple sequence alignment of the Chlamydomonas reinhardtii LCIE protein (SEQ ID NO: 5), LCID/E orthologs of Ettlia oleoabundans (SEQ ID NO: 10, SEQ ID NO: 11, and SEQ ID NO: 12), and the LCID/E ortholog of Zea nicaraguensis (SEQ ID NO: 6) according to CLUSTAL 0(1.2.4).

FIG. 7A-B shows a pairwise alignment of wild-type pyruvate carboxylase of Corynebacterium glutamicum (SEQ ID NO. 78) and a mutated pyruvate carboxylase of Corynebacterium glutamicum that is desensitized to feedback inhibition from aspartic acid (SEQ ID NO. 79) according to CLUSTAL 0(1.2.4), specifically showing the complete sequence of the wild-type pyruvate carboxylase and differences between the mutated pyruvate carboxylase and the wild-type pyruvate carboxylase.

FIG. 8A-I shows a multiple sequence alignment of pyruvate carboxylase of Corynebacterium glutamicum (SEQ ID NO. 78), Bacillus subtilus (SEQ ID NO: 80), Chlamydomonas reinhardtii (SEQ ID NO: 72), Chlorella variabilis (SEQ ID NO: 74), Chlorella sorokiniana (isoform A) (SEQ ID NO: 76), and Chlorella sorokiniana (isoform B) (SEQ ID NO: 77) according to CLUSTAL 0(1.2.4), and also shows positions of mutations of the mutated pyruvate carboxylase of Corynebacterium glutamicum that is desensitized to feedback inhibition from aspartic acid (SEQ ID NO: 79) relative to the other pyruvate carboxylase sequences.

### DETAILED DESCRIPTION OF THE INVENTION

A genetically engineered land plant that expresses an LCID/E protein is disclosed. The genetically engineered land plant comprises a modified gene for the LCID/E protein. The LCID/E protein comprises (i) LCID of Chlamydomonas reinhardtii of SEQ ID NO: 4, (ii) LCIE of Chlamydomonas reinhardtii of SEQ ID NO: 5, or (iii) an algal or plant ortholog of LCID/E. The LCID/E protein is localized to chloroplasts of the genetically engineered land plant based on a plastidial targeting signal. The modified gene for the LCID/E protein comprises (i) a promoter and (ii) a nucleic acid sequence encoding the LCID/E protein. The promoter is non-cognate with respect to the nucleic acid sequence encoding the LCID/E protein. The modified gene for the LCID/E protein is configured such that transcription of the nucleic acid sequence encoding the LCID/E protein is initiated from the promoter and results in expression of the LCID/E protein.

Surprisingly, it has been determined that certain land plants encode orthologs of Chlamydomonas reinhardtii LCID/E. This was surprising because, among other reasons, it is not apparent whether or to what extent LCID and/or LCIE may play roles in carbon-concentrating mechanisms to increase intracellular concentrations of dissolved inorganic carbon. This also was surprising because initial results suggest that only a small number of species of land plants encode LCID/E orthologs, the various species of land plants that encode the LCID/E orthologs appear to be phylogenetically distant from each other, not closely related, and yet the LCID/E orthologs encoded by the various species of land plants appear to be highly similar to LCID/E orthologs of a particular algal species, Ettlia oleoabundans, suggesting the intriguing possibility that the LCID/E orthologs encoded by the various species of land plants may share a common ancestor with the LCID/E orthologs of Ettlia oleoabundans, and/or may have been derived been horizontal gene transfer from Ettlia oleoabundans or a related alga. The result is particularly intriguing because one of the species of land plant is Zea nicaraguensis (also termed teosinte), which is a wild progenitor of the crop plant Zea mays (also termed maize). The result also is intriguing because of various crop plants tested thus far, including Zea mays, none appear to include LCID/E orthologs. To the extent that LCID/E orthologs may play a positive role in carbon-concentrating mechanisms to increase intracellular concentrations of dissolved inorganic carbon, addition of the LCID/E orthologs to crop plants may be a particularly promising approach for enhancing yields.

Without wishing to be bound by theory, it is believed that by genetically engineering a land plant to comprise a modified gene for an LCID/E protein, with the LCID/E protein comprising (i) LCID of Chlamydomonas reinhardtii of SEQ ID NO: 4, (ii) LCIE of Chlamydomonas reinhardtii of SEQ ID NO: 5, or (iii) an algal or plant ortholog of LCID/E, the LCID/E protein being localized to chloroplasts of the genetically engineered land plant based on a plastidial targeting signal, the modified gene for the LCID/E protein comprising (i) a promoter and (ii) a nucleic acid sequence encoding the LCID/E protein, the promoter being non-cognate with respect to the nucleic acid sequence encoding the LCID/E protein, and the modified gene for the LCID/E protein being configured such that transcription of the nucleic acid sequence encoding the LCID/E protein is initiated from the promoter and results in expression of the LCID/E protein, will result in enhanced yield, based for example on an increased CO₂ assimilation rate and/or a decreased transpiration rate of the genetically engineered land plant, in comparison to a reference land plant that does not comprise the modified gene. For example, it is believed that an LCID/E protein may enhance transport of small molecules from or into the chloroplast and/or otherwise alter chloroplast metabolism with respect to small molecules, thereby enhancing rates of carbon fixation. Moreover, it is believed that an LCID/E protein will enhance positive impact of algal and plant CCP1 orthologs with respect to transporting bicarbonate from or into the mitochondria and/or otherwise altering mitochondrial metabolism, thereby enhancing rates of carbon fixation by increasing CO₂ recovery from photorespiration and respiration, or alternatively, increasing transport of small molecules and thereby preventing the accumulation of photorespiratory intermediates that may inhibit photosynthesis. In addition, it is believed that by genetically engineering the land plant to express an LCID/E protein that is localized to chloroplasts in particular, it will be possible to stack expression of the LCID/E protein with expression of other proteins in deliberate and complementary approaches to further enhance yield. In addition, it is believed that by modifying the land plant to express an LCID/E protein of a land plant in particular, it will be possible to generate genetically engineered crops that include only genes, control sequences, and proteins that are proximally derived from land plants, and thus are already generally recognized as safe for human consumption.

As noted, a genetically engineered land plant that expresses an LCID/E protein is disclosed. A land plant is a plant belonging to the plant subkingdom Embryophyta, including higher plants, also termed vascular plants, and mosses, liverworts, and hornworts.

The term “land plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Land plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Elaeis, Saccharum, Avena, Hordeum, Secale, Triticum, Sorghum, Picea, Populus, Camelina, Beta, Solanum, and Carthamus. Preferred land plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, Umbelliferae.

The land plant can be a monocotyledonous land plant or a dicotyledonous land plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Of particular interest are oilseed plants. In oilseed plants of interest the oil is accumulated in the seed and can account for greater than 10%, greater than 15%, greater than 18%, greater than 25%, greater than 35%, greater than 50% by weight of the weight of dry seed. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. In addition to being useful as an industrial oilseed crop, Camelina is a very useful model system for developing new tools and genetically engineered approaches to enhancing the yield of crops in general and for enhancing the yield of seed and seed oil in particular. Demonstrated transgene improvements in Camelina caa then be deployed in major oilseed crops including Brassica species including B. napus (canola), B. rapa, B. juncea, B. carinata, crambe, soybean, sunflower, safflower, oil palm, flax, and cotton.

As will be apparent, the land plant can be a C3 photosynthesis plant, i.e. a plant in which RuBisCO catalyzes carboxylation of ribulose-1,5-bisphosphate by use of CO₂ drawn directly from the atmosphere, such as for example, wheat, oat, and barley, among others. The land plant also can be a C4 plant, i.e. a plant in which RuBisCO catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some examples the genetically engineered land plant is a C3 plant. Also, in some examples the genetically engineered land plant is a C4 plant. Also, in some examples the genetically engineered land plant is a major food crop plant selected from the group consisting of maize, wheat, oat, barley, soybean, millet, sorghum, potato, pulse, bean, tomato, and rice. In some of these examples, the genetically engineered land plant is maize. Also, in some examples the genetically engineered land plant is a forage crop plant selected from the group consisting of silage corn, hay, and alfalfa. In some of these examples, the genetically engineered land plant is silage corn. Also, in some examples the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

The genetically engineered land plant comprises a modified gene for the LCID/E protein. The term “LCID/E protein” means a protein that corresponds to LCD, LCIE, an ortholog of LCD, and/or an ortholog of LCIE.

The LCID/E protein comprises (i) LCD of Chlamydomonas reinhardtii of SEQ ID NO: 4, (ii) LCIE of Chlamydomonas reinhardtii of SEQ ID NO: 5, or (iii) an algal or plant ortholog of LCID/E.

The term “ortholog” means a polynucleotide sequence or polypeptide sequence possessing a high degree of homology, i.e. sequence relatedness, to a subject sequence and being a functional equivalent of the subject sequence, wherein the sequence that is orthologous is from a species that is different than that of the subject sequence. Homology may be quantified by determining the degree of identity and/or similarity between the sequences being compared.

As used herein, “percent homology” of two polypeptide sequences is the percent identity over the length of the entire sequence determined using EMBOSS Needle Pairwise Sequence Alignment (PROTEIN) tool using default settings (matrix: BLOSUM62; gap open: 10; gap extend: 0.5; output format: pair; end gap penalty: false; end gap open: 10; end gap extend: 0.5) (website: ebi.ac.uk/Tools/psa/emboss_needle/). The percentage of sequence identity between two polynucleotide sequences or two polypeptide sequences can also be determined by using various software packages, such as the ALIGNX alignment function of the Vector NTI software package (Vector NTI Advance, Version 11.5.3, ThermoFisher), which uses the Clustal W algorithm.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Many other polypeptides will meet the same criteria.

For reference, as discussed above LCID and LCIE are members of a family of low carbon inducible proteins that have been identified in the algal species Chlamydomonas reinhardtii. LCID of Chlamydomonas reinhardtii has an amino acid sequence in accordance with SEQ ID NO: 4. LCIE of Chlamydomonas reinhardtii has an amino acid sequence in accordance with SEQ ID NO: 5. Accordingly, an algal or plant ortholog of LCID/E is a polypeptide sequence possessing a high degree of sequence relatedness to LCID of Chlamydomonas reinhardtii of SEQ ID NO: 4 and/or LCIE of Chlamydomonas reinhardtii of SEQ ID NO: 5 and being a functional equivalent thereof, in the case of an algal ortholog of LCID/E being derived from a eukaryotic alga, and in the case of a plant ortholog of LCID/E being derived from a land plant.

Accordingly, the LCID/E protein can be derived, for example, from a eukaryotic alga. For reference, Chlamydomonas reinhardtii is a eukaryotic alga. In contrast to a land plant, a eukaryotic alga is an aquatic plant, ranging from a microscopic unicellular form, e.g. a single-cell alga, to a macroscopic multicellular form, e.g. a seaweed, that includes chlorophyll a and, if multicellular, a thallus not differentiated into roots, stem, and leaves, and that is classified as chlorophyta (also termed green algae), rhodophyta (also termed red algae), or phaeophyta (also termed brown algae). Eukaryotic algae include, for example, single-cell algae, including the chlorophyta Chlamydomonas reinhardtii, Chlorella sorokiniana, and Chlorella variabilis. Eukaryotic algae also include, for example, seaweed, including the chlorophyta Ulva lactuca (also termed sea lettuce) and Enteromorpha (Ulva) intenstinalis (also termed sea grass), the rhodophyta Chondrus crispus (also termed Irish moss or carrigeen), Porphyra umbilicalis (also termed nori), and Palmaria palmata (also termed dulse or dillisk), and the phaeophyta Ascophyllum nodosum (also termed egg wrack), Laminaria digitata (also termed kombu/konbu), Laminaria saccharina (also termed royal or sweet kombu), Himanthalia elongata (also termed sea spaghetti), and Undaria pinnatifida (also termed wakame). Eukaryotic algae also include, for example, additional chlorophyta such as Gonium pectorals, Volvox carteri f. nagariensis, and Ettlia oleoabundans. The source eukaryotic alga from which the LCID/E protein is derived can be a eukaryotic alga as described above, i.e. a eukaryotic alga that includes an LCID/E protein. Examples of eukaryotic alga that include an LCID/E protein include Chlamydomonas reinhardtii and Ettlia oleoabundans, among others.

Also accordingly, the LCID/E protein also can be derived, for example, from a land plant. The source land plant from which the LCID/E protein is derived can be a land plant as described above, i.e. a plant belonging to the plant subkingdom Embryophyta, that includes an LCID/E protein. Examples of land plants that appear to include an LCID/E protein, based on TBLASTN searches and the presence of at least partial sequences, include Zea nicaraguensis, Cosmos bipinnatus, Arachis hypogaea var. vulgaris, Solanum prinophyllum, Colobanthus quitensis, Poa pratensis, Nymphoides peltata, Camellia sinensis, Picea glauca, Triticum polonicum, Araucaria cunninghamii, Pohlia nutans, and Elodea nuttallii. Examples of land plants that appear to include an LCID/E protein, based on TBLASTN searches and the presence of apparently complete sequences, include Zea nicaraguensis, Cosmos bipinnatus, and Nymphoides peltata.

In some examples the source land plant is a different type of land plant than the genetically engineered land plant. In accordance with these examples, the LCID/E protein can be heterologous with respect to the genetically engineered land plant. By this it is meant that the particular LCID/E protein derived from the source land plant is not normally encoded, expressed, or otherwise present in land plants of the type from which the genetically engineered land plant is derived. This can be because land plants of the type from which the genetically engineered land plant is derived do not normally encode, express, or otherwise include the particular LCID/E protein, and this can be so whether or not the land plants normally express a different, endogenous LCID/E protein. The genetically engineered land plant expresses the particular LCID/E protein based on comprising the modified gene for the LCID/E protein. Accordingly, the modified gene can be used to accomplish modified expression of the LCID/E protein, and particularly increased expression of ortholog(s) of LCID/E, including the LCID/E protein and any endogenous LCID/E proteins.

Also in some examples the source land plant is the same type of land plant as the genetically engineered land plant. In accordance with these examples, the LCID/E protein can be homologous with respect to the genetically engineered land plant. By this it is meant that the particular LCID/E protein is normally encoded, and may normally be expressed, in land plants of the type from which the genetically engineered land plant is derived. In accordance with these examples, the land plant can be genetically engineered to include additional copies of a gene for the LCID/E protein and/or to express an endogenous copy a gene for the LCID/E protein at higher levels and/or in a tissue-preferred manner based on modification and/or replacement of a promoter for the endogenous copy of the gene. Again, the genetically engineered land plant expresses the particular LCID/E protein based on comprising the modified gene for the LCID/E protein, resulting in modified expression of the LCID/E protein, and particularly increased expression of ortholog(s) of LCID/E.

As discussed above, it is believed that an LCID/E protein may enhance transport of small molecules from or into the chloroplast and/or otherwise alter chloroplast metabolism with respect to small molecules, thereby enhancing rates of carbon fixation. Accordingly, the LCID/E protein may be a protein that transports small molecules by any transport mechanism. Classes of small molecule transport proteins include anion exchangers and Na⁺/HCO₃⁻¹ symporters. The LCID/E protein also may be a protein that otherwise alters chloroplast metabolism with respect to small molecules. Increased transport and/or alteration of metabolism of small molecules may prevent their buildup which might otherwise inhibit photosynthesis. An additional possibility is that the LCID/E protein serves as a guide by binding to other proteins such as CCP1 and directing them to the chloroplast. In this way, LCID/E could facilitate the simultaneous localization of proteins such as CCP1 to both the mitochondrion and chloroplast so that complementary transport functions could occur at both organelles.

The LCID/E protein is localized to chloroplasts of the genetically engineered land plant based on a plastidial targeting signal. The LCID/E protein can be localized to chloroplast for example based on being encoded by DNA present in the nucleus of a plant cell, synthesized in the cytosol of the plant cell, targeted to the chloroplast of the plant cell, and inserted into outer membranes and/or inner membranes of the chloroplast. A plastidial targeting signal is a portion of a polypeptide sequence that targets the polypeptide sequence to chloroplasts. In some examples, the plastidial targeting signal is intrinsic to the LCID/E protein. A plastidial targeting signal that is intrinsic to the LCID/E protein is a plastidial targeting signal that is integral to the LCID/E protein, e.g. based on occurring naturally at the N-terminal end of the LCID/E protein or in discrete segments along the LCID/E protein. Also in some examples, the plastidial targeting signal is heterologous with respect to the LCID/E protein.

Suitable LCID/E proteins can be identified, for example, based on searching databases of polynucleotide sequences or polypeptide sequences for orthologs of LCD of Chlamydomonas reinhardtii of SEQ ID NO: 4 and/or LCIE of Chlamydomonas reinhardtii of SEQ ID NO: 5, wherein the polynucleotide sequences or polypeptide sequences are derived from eukaryotic algae and/or land plants, in view of the disclosure herein, as discussed below. Such searches can be carried out, for example, by use of BLAST, e.g. tblastn, and databases including translated polynucleotides, whole genome shotgun sequences, and/or transcriptome assembly sequences, among other sequences and databases. Potential orthologs of LCID/E may be identified, for example, based on percentage of identity and/or percentage of similarity, with respect to polypeptide sequence, of individual sequences in the databases in comparison to LCID and/or LCIE of Chlamydomonas reinhardtii. For example, potential orthologs of LCID/E may be identified based on percentage of identity of an individual sequence in a database and LCID of Chlamydomonas reinhardtii of SEQ ID NO: 4 and/or LCIE of Chlamydomonas reinhardtii of SEQ ID NO: 5 of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from a eukaryotic alga or a land plant. Also for example, potential orthologs of LCID/E may be identified based on percentage of similarity of an individual sequence in a database and LCID of Chlamydomonas reinhardtii of SEQ ID NO: 4 and/or LCIE of Chlamydomonas reinhardtii of SEQ ID NO: 5 of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from a eukaryotic alga or a land plant. Also for example, potential orthologs of LCID/E may be identified based on both percentage of identity of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95%, and percentage of similarity of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from a eukaryotic alga or a land plant.

Following identification of an LCID/E protein, genetic engineering of a land plant to express the LCID/E protein can be carried out by methods that are known in the art, as discussed in detail below.

The genetically engineered land plant can be a genetically engineered land plant that includes no heterologous proteins, e.g. wherein the LCID/E protein is homologous with respect to the genetically engineered land plant, or only one heterologous protein, e.g. wherein the only heterologous plant protein that the genetically engineered land plant comprises is the LCID/E protein.

Considering the LCID/E protein in more detail, the LCID/E protein can correspond, for example, to an LCID/E protein selected from among specific polypeptide sequences of source eukaryotic algae and/or source land plants. As noted above, the LCID/E protein can be identified based on homology to LCID of Chlamydomonas reinhardtii of SEQ ID NO: 4 and/or LCIE of Chlamydomonas reinhardtii of SEQ ID NO: 5. Regarding source land plants in particular, exemplary LCID/E proteins identified this way include (a) an LCID/E protein of Zea nicaraguensis, (b) an LCID/E protein of Cosmos bipinnatus, or (c) an LCID/E protein of Nymphoides peltata. Thus, for example, the LCID/E protein can comprise an LCID/E protein of Zea nicaraguensis. Also for example, LCID/E proteins identified this way include (a) an LCID/E protein of Zea nicaraguensis of SEQ ID NO: 6, (b) an LCID/E protein of Cosmos bipinnatus of SEQ ID NO: 7, or (c) an LCID/E protein of Nymphoides peltata of SEQ ID NO: 8. Thus, for example, the LCID/E protein can comprise an LCID/E protein of Zea nicaraguensis of SEQ ID NO: 6.

The LCID/E protein also can correspond to an LCID/E protein including specific structural features and characteristics shared among various orthologs of LCID/E. Such structural features and characteristics shared among the various orthologs of LCID/E, namely the LCID/E protein of Zea nicaraguensis of SEQ ID NO: 6, the LCID/E protein of Cosmos bipinnatus of SEQ ID NO: 7, and the LCID/E protein of Nymphoides peltata of SEQ ID NO: 8, include (i) (a) a glutamate residue at position 161, (b) a cysteine residue at position 189, (c) a cysteine residue at position 241, (d) an aspartate residue at position 310, and (e) a glutamate residue at position 312, with numbering of positions relative to LCID of Chlamydomonas reinhardtii of SEQ ID NO: 4, and (ii) an overall identity of at least 15%. These noted amino acid residues are conserved among LCID of Chlamydomonas reinhardtii of SEQ ID NO: 4, LCIE of Chlamydomonas reinhardtii of SEQ ID NO: 5, LCID/E protein of Ettlia oleoabundans of SEQ ID NO: 10 and SEQ ID NO: 12, LCID/E protein of Zea nicaraguensis of SEQ ID NO: 6, LCID/E protein of Cosmos bipinnatus of SEQ ID NO: 7, and LCID/E protein of Nymphoides peltata of SEQ ID NO: 8. Conservation of the noted amino acid residues, in combination with an overall identity of at least 15%, suggests a structure/function relationship shared among such LCID/E proteins. Thus, for example, the LCID/E protein can be an ortholog of LCID/E of Chlamydomonas reinhardtii based on comprising: (i) (a) a glutamate residue at position 161, (b) a cysteine residue at position 189, (c) a cysteine residue at position 241, (d) an aspartate residue at position 310, and (e) a glutamate residue at position 312, with numbering of positions relative to LCID of Chlamydomonas reinhardtii of SEQ ID NO: 4, and (ii) an overall identity of at least 15%.

The LCID/E protein also can correspond to an LCID/E protein including additional specific structural features and characteristics shared among orthologs of LCID/E. For example, the LCID/E protein can be an ortholog of LCID/E based on comprising: (i) (a) an asparagine residue at position 233, (b) a lysine residue at position 322, and (c) a glutamine residue at position 405, with numbering of positions relative to LCID of Chlamydomonas reinhardtii of SEQ ID NO: 4, and (ii) an overall identity of at least 15%. These noted amino acid residues are conserved among LCID of Chlamydomonas reinhardtii of SEQ ID NO: 4, LCIE of Chlamydomonas reinhardtii of SEQ ID NO: 5, LCID/E protein of Ettlia oleoabundans of SEQ ID NO: 11, and LCID/E protein of Zea nicaraguensis of SEQ ID NO: 6.

The LCID/E protein also can correspond to an LCID/E protein including LCID/E signature sequences shared specifically among an algal LCID/E protein of Ettlia oleoabundans and an LCID/E protein of Zea nicaraguensis. For example, the LCID/E protein can be an ortholog of LCID/E based on comprising: (i) one or more LCID/E signature sequences of (a) FSFPHI (SEQ ID NO: 13) at position 213-218, (b) ACGAL (SEQ ID NO: 14) at position 240-244, (c) ADYAV (SEQ ID NO: 15) at position 324-328, or (d) TGVQIHNW (SEQ ID NO: 16) at position 330-337, with numbering of positions relative to LCID of Chlamydomonas reinhardtii of SEQ ID NO: 4, and (ii) an overall identity of at least 60%. These noted LCID/E signature sequences also are conserved specifically among algal LCID/E protein of Ettlia oleoabundans of SEQ ID NO: 10, SEQ ID NO: 11, and SEQ ID NO: 12, and an LCID/E protein of Zea nicaraguensis of SEQ ID NO: 6.

The LCID/E protein also can correspond to an LCID/E protein that does not differ in any biologically significant way from a wild-type LCID/E protein. As noted above, the LCID/E protein is localized to chloroplasts of the genetically engineered land plant based on a plastidial targeting signal. In some examples, the plastidial targeting signal is intrinsic to the LCID/E protein. Also in some examples, the LCID/E protein is heterologous with respect to the genetically engineered land plant. In some examples, the LCID/E protein also does not include any other modifications that might result in the LCID/E protein differing in a biologically significant way from a wild-type LCID/E protein. Thus, for example the LCID/E protein can consist essentially of an amino acid sequence that is identical to that of a wild-type LCID/E protein. The corresponding genetically engineered land plant will provide advantages, e.g. again in terms of lower risk of harmful effects with respect to use of the genetically engineered land plant as a food crop, a forage crop, or an oilseed crop.

The modified gene for the LCID/E protein comprises (i) a promoter and (ii) a nucleic acid sequence encoding the LCID/E protein.

The promoter is non-cognate with respect to the nucleic acid sequence encoding the LCID/E protein. A promoter that is non-cognate with respect to a nucleic acid sequence means that the promoter is not naturally paired with the nucleic acid sequence in organisms from which the promoter and/or the nucleic acid sequence are derived. Instead, the promoter has been paired with the nucleic acid sequence based on use of recombinant DNA techniques to create a modified gene. Accordingly, for example in this case, the promoter is not naturally paired with the nucleic acid sequence in a source eukaryotic alga or a source land plant, i.e. a eukaryotic alga or land plant from which the nucleic acid sequence encoding the LCID/E protein had been derived, nor in the organism from which the promoter has been derived, whether that organism is the source eukaryotic alga, source land plant, or another organism. Instead, the promoter has been paired with the nucleic acid sequence based on use of recombinant DNA techniques to create the modified gene.

The modified gene for the LCID/E protein is configured such that transcription of the nucleic acid sequence encoding the LCID/E protein is initiated from the promoter and results in expression of the LCID/E protein. Accordingly, in the context of the modified gene, the promoter functions as a promoter of transcription of the nucleic acid sequence, and thus of expression of the LCID/E protein. In some examples, the expression of the LCID/E protein is higher in the genetically engineered land plant than in a corresponding plant that does not include the modified gene.

In some examples, the promoter is a constitutive promoter. In some examples, the promoter is a seed-specific promoter. In some examples, the modified gene is integrated into genomic DNA of the genetically engineered land plant. In some examples, the modified gene is stably expressed in the genetically engineered land plant. In some examples the nucleic acid sequence encodes a wild-type LCID/E protein. In some examples, the nucleic acid sequence encodes a variant, modified, mutant, or otherwise non-wild-type LCID/E protein. These exemplary features, and others, of the promoter, the nucleic acid sequence, and the modified gene are discussed in detail below.

The genetically engineered land plant also can be a genetically engineered land plant that expresses nucleic acid sequences encoding LCID/E proteins in both a seed-specific and a constitutive manner, wherein the nucleic acid sequences encoding the LCID/E proteins may be the same or different nucleic acid sequences, e.g. from the same source land plant or from different source land plants. In some examples the genetically engineered land plant (i) expresses the LCID/E protein in a seed-specific manner, and (ii) expresses another LCID/E protein constitutively, the other LCID/E protein also corresponding to an ortholog of LCID/E derived from a source eukaryotic alga or source land plant.

The genetically engineered land plant can have a CO₂ assimilation rate that is higher than for a corresponding reference land plant not comprising the modified gene. For example, the genetically engineered land plant can have a CO₂ assimilation rate that is at least 5% higher, at least 10% higher, at least 20% higher, or at least 40% higher, than for a corresponding reference land plant that does not comprise the modified gene.

The genetically engineered land plant also can have a transpiration rate that is lower than for a corresponding reference land plant not comprising the modified gene. For example, the genetically engineered land plant can have a transpiration rate that is at least 5% lower, at least 10% lower, at least 20% lower, or at least 40% lower, than for a corresponding reference land plant that does not comprise the modified gene.

The genetically engineered land plant also can have a seed yield that is higher than for a corresponding reference land plant not comprising the modified gene. For example, the genetically engineered land plant can have a seed yield that is at least 5% higher, at least 10% higher, at least 20% higher, at least 40% higher, at least 60% higher, or at least 80% higher, than for a corresponding reference land plant that does not comprise the modified gene.

As noted above, following identification of an LCID/E protein of a source land plant, genetic engineering of a land plant to express the LCID/E protein can be carried out by methods that are known in the art, for example as follows.

DNA constructs useful in the methods described herein include transformation vectors capable of introducing transgenes or other modified nucleic acid sequences into land plants. As used herein, “genetically engineered” refers to an organism in which a nucleic acid fragment containing a heterologous nucleotide sequence has been introduced, or in which the expression of a homologous gene has been modified, for example by genome editing. Transgenes in the genetically engineered organism are preferably stable and inheritable. Heterologous nucleic acid fragments may or may not be integrated into the host genome.

Several plant transformation vector options are available, including those described in Gene Transfer to Plants, 1995, Potrykus et al., eds., Springer-Verlag Berlin Heidelberg New York, Genetically engineered Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof. See, for example, U.S. Pat. No 5,639,949.

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. See, for example, U.S. Pat. No. 5,639,949. Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online March 2, 2014; doi;10.1038/nbt.2842) is particularly useful for editing plant genomes to modulate the expression of homologous genes encoding enzymes. All that is required to achieve a CRISPR/Cas edit is a Cas enzyme, or other CRISPR nuclease (Murugan et al. Mol Cell 2017, 68:15), and a single guide RNA (sgRNA) as reviewed extensively by others (Belhag et al. Curr Opin Biotech 2015, 32: 76; Khandagale and Nadaf, Plant Biotechnol Rep 2016, 10:327). Several examples of the use of this technology to edit the genomes of plants have now been reported (Belhaj et al. Plant Methods 2013, 9:39); Zhang et al. Journal of Genetics and Genomics 2016, 43: 251).

TALENs (transcriptional activator-like effector nucleases) or meganucleases can also be used for plant genome editing (Malzahn et al., Cell Biosci, 2017, 7:21).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG) , electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described [Evans et al., in Handbook of Plant Cell Culture, Vol 1, (Macmillan Publishing Co., New York, 1983); Vasil, IK in Cell Culture and Somatic Cell Genetics (Academic, Orlando, 1984)].

Recombinase technologies which are useful for producing the disclosed genetically engineered plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain genetically engineered plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant 1 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Genetically engineered Res. 9: 471-476). Other plants that have successfully been transformed by in planta methods include rapeseed and radish (vacuum infiltration, Ian and Hong, 2001, Genetically engineered Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Genetically engineered plants can be produced using conventional techniques to express any genes of interest in plants or plant cells (Methods in Molecular Biology, 2005, vol. 286, Genetically engineered Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant 12: 255-265; Kawamata et al., 1997, Plant Cell Physiol. 38: 792-803; Hansen et al., 1997, Mol. Gen. Genet. 254: 337-343; Russell et al., 1997, Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Chemical-regulated promoters can be used to modulate the expression of a gene in a plant through the application of an exogenous chemical regulator.

Specific exemplary promoters useful for expression of genes in dicots and monocots are provided in TABLE 1 and TABLE 2, respectively.

TABLE 1
Promoters useful for expression of genes in dicots.

| | | Native | |
|---|---|---|---|
| | | organism | |
| Gene/Promoter | Expression | of promoter | Gene ID* |
| Hsp70 | Constitutive | Glycine max | Glyma.02G093200 |
| | | | (SEQ ID NO: 34) |
| Chlorophyll A/B | Constitutive | Glycine max | Glyma.08G082900 |
| Binding Protein | | | (SEQ ID NO: 35) |
| (Cab5) | | | |
| Pyruvate | Constitutive | Glycine max | Glyma.06G252400 |
| phosphate dikinase | | | (SEQ ID NO: 36) |
| (PPDK) | | | |
| Actin | Constitutive | Glycine max | Glyma.19G147900 |
| | | | (SEQ ID NO: 37) |
| ADP-glucose | Seed specific | Glycine max | Glyma.04G011900 |
| pyrophosphorylase | | | (SEQ ID NO: 38) |
| (AGPase) | | | |
| Glutelin C | Seed specific | Glycine max | Glyma.03G163500 |
| (GluC) | | | (SEQ ID NO: 39) |
| β-fructofuranosidase | Seed specific | Glycine max | Glyma.17G227800 |
| insoluble isoenzyme 1 | | | (SEQ ID NO: 40) |
| (CIN1) | | | |
| MADS-Box | Cob specific | Glycine max | Glyma.04G257100 |
| | | | (SEQ ID NO: 41) |
| Glycinin | Seed specific | Glycine max | Glyma.03G163500 |
| (subunit G1) | | | (SEQ ID NO: 42) |
| oleosin isoform A | Seed specific | Glycine max | Glyma.16G071800 |
| | | | (SEQ ID NO: 43) |
| Hsp70 | Constitutive | Brassica napus | BnaA09g05860D |
| Chlorophyll A/B | Constitutive | Brassica napus | BnaA04g20150D |
| Binding Protein | | | |
| (Cab5) | | | |
| Pyruvate | Constitutive | Brassica napus | BnaA01g18440D |
| phosphate dikinase | | | |
| (PPDK) | | | |
| Actin | Constitutive | Brassica napus | BnaA03g34950D |
| ADP-glucose | Seed specific | Brassica napus | BnaA06g40730D |
| pyrophosphorylase | | | |
| (AGPase) | | | |
| Glutelin C | Seed specific | Brassica napus | BnaA09g50780D |
| (GluC) | | | |
| β-fructofuranosidase | Seed specific | Brassica napus | BnaA04g05320D |
| insoluble isoenzyme 1 | | | |
| (CIN1) | | | |
| MADS-Box | Cob specific | Brassica napus | BnaA05g02990D |
| Glycinin | Seed specific | Brassica napus | BnaA01g08350D |
| (subunit G1) | | | |
| oleosin isoform A | Seed specific | Brassica napus | BnaC06g12930D |
| 1.7S napin | Seed specific | Brassica napus | BnaA01g17200D |
| (napA) | | | |

*Gene ID includes sequence information for coding regions as well as associated promoters, 5′ UTRs, and 3′ UTRs and are available at Phytozome (see JGI website phytozome.jgi.doe.gov/pz/portal.html).

TABLE 2
Promoters useful for expression of genes in monocots,
including maize and rice.

| Gene/Promoter | Expression | Rice* | Maize* |
|---|---|---|---|
| Hsp70 | Constitutive | LOC_Os05g38530 | GRMZM2G |
| | | (SEQ ID NO: 26) | 310431 |
| | | | (SEQ ID |
| | | | NO: 17) |
| Chlorophyll A/B | Constitutive | LOC_Os01g41710 | AC207722.2_FG009 |
| Binding Protein | | (SEQ ID NO: 27) | (SEQ ID |
| (Cab5) | | | NO: 18) |
| | | | GRMZM2G |
| | | | 351977 |
| | | | (SEQ ID |
| | | | NO: 19) |
| Pyruvate phosphate | Constitutive | LOC_Os05g33570 | GRMZM2G |
| dikinase | | (SEQ ID NO: 28) | 306345 |
| (PPDK) | | | (SEQ ID |
| | | | NO: 20) |
| Actin | Constitutive | LOC_Os03g50885 | GRMZM2G |
| | | (SEQ ID NO: 29) | 047055 |
| | | | (SEQ ID |
| | | | NO: 21) |
| Hybrid cab5/hsp70 | Constitutive | N/A | SEQ ID NO: |
| intron promoter | | | 22 |
| ADP-glucose | Seed | LOC_Os01g44220 | GRMZM2G |
| pyrophos-phorylase | specific | (SEQ ID NO: 30) | 429899 |
| (AGPase) | | | (SEQ ID |
| | | | NO: 23) |
| Glutelin C (GluC) | Seed | LOC_Os02g25640 | N/A |
| | specific | (SEQ ID NO: 31) | |
| β-fructofuranosidase | Seed | LOC_Os02g33110 | GRMZM2G |
| insoluble | specific | (SEQ ID NO: 32) | 139300 |
| isoenzyme 1 | | | (SEQ ID |
| (CIN1) | | | NO: 24) |
| MADS-Box | Cob specific | LOC_Os12g10540 | GRMZM2G |
| | | (SEQ ID NO: 33) | 160687 |
| | | | (SEQ ID |
| | | | NO: 25 |

*Gene ID includes sequence information for coding regions as well as associated promoters., 5′ UTRs, and 3′ UTRs and are available at Phytozome (see JGI website phytozome.jgi.doe.gov/pz/portal.html).

Certain embodiments use genetically engineered plants or plant cells having multi-gene expression constructs harboring more than one transgene and promoter. The promoters can be the same or different.

Any of the described promoters can be used to control the expression of one or more of genes, their homologs and/or orthologs as well as any other genes of interest in a defined spatiotemporal manner.

Nucleic acid sequences intended for expression in genetically engineered plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of genetically engineered plants that express a recombinant gene(s) may have different levels of gene expression. The variable gene expression is due to multiple factors including multiple copies of the recombinant gene, chromatin effects, and gene suppression. Accordingly, a phenotype of the genetically engineered plant may be measured as a percentage of individual plants within a population. The yield of a plant can be measured simply by weighing. The yield of seed from a plant can also be determined by weighing. The increase in seed weight from a plant can be due to a number of factors, including an increase in the number or size of the seed pods, an increase in the number of seed and/or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A genetically engineered plant can be produced by selection of transformed seeds or by selection of transformed plant cells and subsequent regeneration.

In some embodiments, the genetically engineered plants are grown (e.g., on soil) and harvested. In some embodiments, above ground tissue is harvested separately from below ground tissue. Suitable above ground tissues include shoots, stems, leaves, flowers, grain, and seed. Exemplary below ground tissues include roots and root hairs. In some embodiments, whole plants are harvested and the above ground tissue is subsequently separated from the below ground tissue.

Genetic constructs may encode a selectable marker to enable selection of transformation events. There are many methods that have been described for the selection of transformed plants (for review see (Miki et al., Journal of Biotechnology, 2004, 107, 193-232) and references incorporated within). Selectable marker genes that have been used extensively in plants include the neomycin phosphotransferase gene nptll (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 Al describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of genetically engineered plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296). Improved versions of many of the fluorescent proteins have been made for various applications. It will be apparent to those skilled in the art how to use the improved versions of these proteins, including combinations, for selection of transformants.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162).

The genetically engineered land plant that expresses an LCID/E protein, as disclosed, can be further modified for further enhanced yield too.

For example, the genetically engineered land plant can express one or more mitochondrial transporter proteins that are also expressed as members of carbon-concentrating mechanisms of eukaryotic algae, as well as expressing an LCID/E protein. In some examples, the mitochondrial transporter protein is a CCP1 mitochondrial transporter protein. In some examples the mitochondrial transporter protein is expressed under the control of plant promoters which may be constitutive, tissue specific, or seed specific. Such genetically engineered plants are expected to have further enhanced yield as compared to plants not expressing the mitochondrial transporter protein, the LCID/E protein, or both. For example, such genetically engineered plants may have improved performance, such as increased CO₂ fixation rates, reduced transpiration, and/or increased biomass and/or seed yield.

Thus, in some examples the genetically engineered land plant further expresses a CCP1 mitochondrial transporter protein. In these examples, the genetically engineered land plant comprises a modified gene for the CCP1 mitochondrial transporter protein. The CCP1 mitochondrial transporter protein comprises: (i) CCP1 of Chlamydomonas reinhardtii of SEQ ID NO: 9 or (ii) an ortholog of CCP1. The ortholog of CCP1 can be, for example, an algal CCP1 ortholog, such as a CCP1 ortholog of Gonium pectorals (e.g. SEQ ID NO: 44 or SEQ ID NO: 45), Volvox carteri f. nagariensis (e.g. SEQ ID NO: 46), Volvox carteri (e.g. SEQ ID NO: 47), Ettlia oleoabundans (e.g. SEQ ID NO: 48), Chlorella sorokiniana (e.g. SEQ ID NO: 49), Chlorella variabilis (e.g. SEQ ID NO: 50, SEQ ID NO: 51, SEQ ID NO: 52, or SEQ ID NO: 54), or Chondrus crispus (e.g. SEQ ID NO: 53, SEQ ID NO: 55, or SEQ ID NO: 56). The ortholog of CCP1 also can be, for example, a plant CCP1 ortholog, such as a CCP1 ortholog of Erigeron breviscapus (e.g. SEQ ID NO: 57), Zea nicaraguensis (e.g. SEQ ID NO: 58), Poa pratensis (e.g. SEQ ID NO: 59), Cosmos bipinnatus (e.g. SEQ ID NO: 60), Glycine max (e.g. SEQ ID NO: 61), Zea mays (e.g. SEQ ID NO: 62), Oryza sativa (e.g. SEQ ID NO: 63), Triticum aestivum (e.g. SEQ ID NO: 64), Sorghum bicolor (e.g. SEQ ID NO: 65), or Solanum tuberosum (e.g. SEQ ID NO: 66).

The CCP1 mitochondrial transporter protein is localized to mitochondria of the genetically engineered land plant based on a mitochondrial targeting signal. The modified gene for the CCP1 mitochondrial transporter protein comprises (i) another promoter and (ii) a nucleic acid sequence encoding the CCP1 mitochondrial transporter protein. The other promoter is non-cognate with respect to the nucleic acid sequence. The modified gene for the CCP1 mitochondrial transporter protein is configured such that transcription of the nucleic acid sequence encoding the CCP1 mitochondrial transporter protein is initiated from the other promoter and results in expression of the CCP1 mitochondrial transporter protein.

Also for example, the genetically engineered land plant can be transformed with transgenic polynucleotides encoding one or more metabolic enzymes. In some examples, the metabolic enzyme includes pyruvate carboxylase. In some examples the metabolic enzyme is expressed under the control of plant promoters which may be constitutive, tissue specific, or seed specific. Such genetically engineered plants also are expected to have further enhanced yield as compared to plants not expressing the metabolic enzyme. For example, such genetically engineered plants also may have improved performance, such as increased CO₂ fixation rates, reduced transpiration, and/or increased biomass and/or seed yield.

Thus, in some examples the genetically engineered land plant further expresses a pyruvate carboxylase. In these examples, the genetically engineered land plant comprises a modified gene for the pyruvate carboxylase. The pyruvate carboxylase can comprise, for example, a bacterial pyruvate carboxylase, such as a pyruvate carboxylase of Corynebacterium glutamicum of SEQ ID NO. 78 or Bacillus subtilus of SEQ ID NO: 80, among others. Also, the pyruvate carboxylase can comprise, for example, an algal pyruvate carboxylase, such as a pyruvate carboxylase of Chlamydomonas reinhardtii of SEQ ID NO: 72, Chlorella variabilis of SEQ ID NO: 74, or Chlorella sorokiniana of SEQ ID NO: 76 or SEQ ID NO: 77, among others. Also, the pyruvate carboxylase can comprise, for example, a pyruvate carboxylase that is desensitized to feedback inhibition from aspartic acid. The pyruvate carboxylase that is desensitized to feedback inhibition from aspartic acid can be desensitized, for example, based on comprising one or more of, i.e. one, two, three, four, five, or six of: (a) an aspartate residue at position 153, (b) a serine residue at position 182, (c) a serine residue at position 206, (d) an arginine residue at position 227, (e) a glycine residue at position 455, or (f) a glutamate residue at position 1120, with numbering of positions relative to pyruvate carboxylase of Corynebacterium glutamicum of SEQ ID NO. 78. Thus, for example, the pyruvate carboxylase that is desensitized to feedback inhibition from aspartic acid can be desensitized based on making substitutions in a bacterial pyruvate carboxylase, e.g. of Corynebacterium glutamicum or Bacillus subtilus, among others, or an algal pyruvate carboxylase, e.g. of Chlamydomonas reinhardtii, Chlorella variabilis, or Chlorella sorokiniana, among others. Also, the pyruvate carboxylase that is desensitized to feedback inhibition from aspartic acid can comprise, for example, a mutated pyruvate carboxylase of Corynebacterium glutamicum of SEQ ID NO. 79.

The modified gene for the pyruvate carboxylase comprises (i) a further promoter and (ii) a nucleic acid sequence encoding the pyruvate carboxylase. The further promoter is non-cognate with respect to the nucleic acid sequence encoding the pyruvate carboxylase. The modified gene for the pyruvate carboxylase is configured such that transcription of the nucleic acid sequence encoding the pyruvate carboxylase is initiated from the further promoter and results in expression of the pyruvate carboxylase.

The pyruvate carboxylase can be, for example, heterologous with respect to the genetically engineered land plant. The further promoter can be, for example, a constitutive promoter, a leaf-specific promoter, or a seed-specific promoter, among other promoters. With respect to a seed-specific promoter, the pyruvate carboxylase can be, for example, expressed in cytosol and/or targeted to plastid.

### EXAMPLES

### Example 1. Identification of LCID and LCIE Genes and Orthologs from Eukaryotic Algae to Improve Crop Performance or Increase Crop Yield

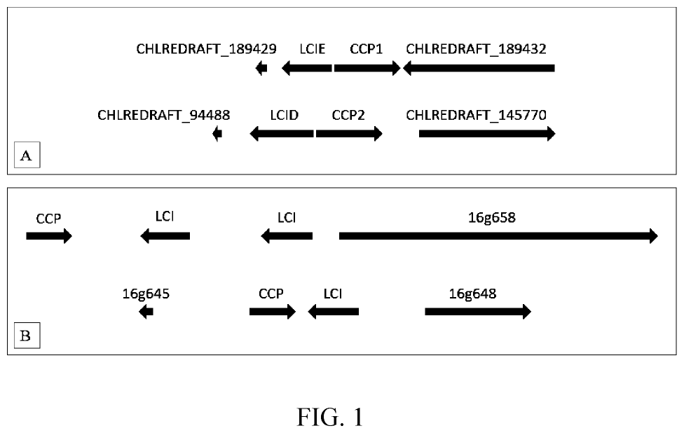

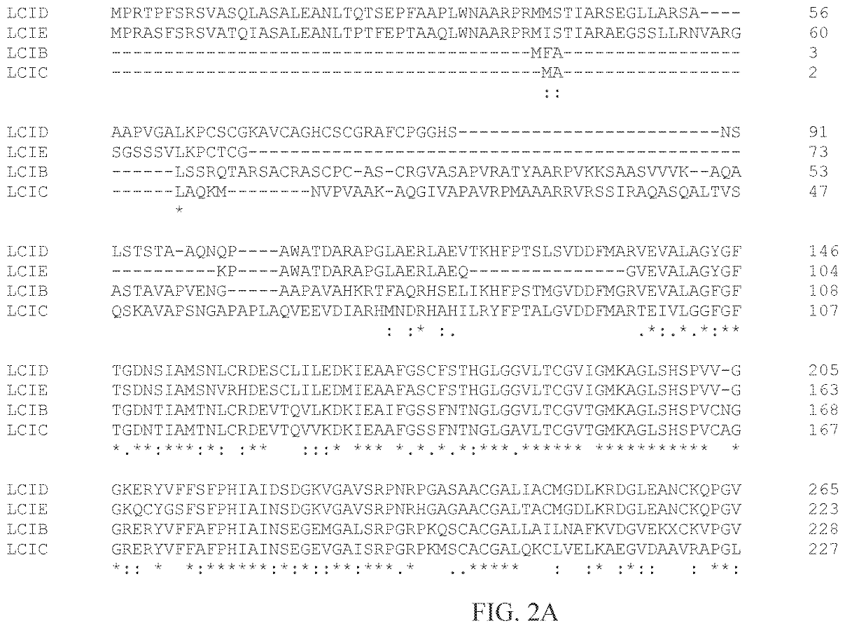

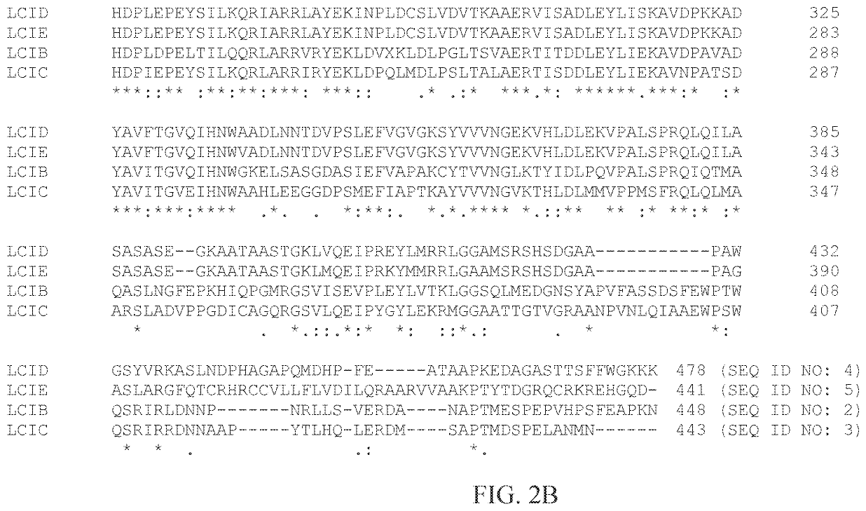

In Chlamydomonas reinhardtii, the CCP1 and CCP2 genes are each adjacent to a gene whose function remains unknown, namely the LCIE and LCID genes, respectively (FIG. 1A). This type of arrangement also occurs in Gonium pectorals (FIG. 1B). Furthermore, other algae that contain CCP1-like genes, such as Volvox carteri f. nagariensis and Chlorella sorokiniana, also contain LCID/E genes. In V. carteri the two are not adjacent, while in C. sorokiniana the relative locations are currently unclear, as the genomic sequence information was gathered from RNA sequencing. The gene pairs CCP1-LCIE and CCP2-LCID in C. reinhardtii may be co-regulated, and their expression profiles are indeed similar (Spalding, 2009, J. Exp. Bot. 59(7):1463-73; Yamano et al., 2008, Plant Physiology 147(1):340-54). The proximity and co-regulation of CCP1/CCP2 and LCIE/LCID may have a biological significance, for example that LCIE or LCID may complement or enhance the function of CCP1 or CCP2 or their orthologs. Standard BLAST searches using C. reinhardtii LCIE or LCID as a query sequence reveal a large number of highly similar proteins, including the LCIB and LCIC proteins within C. reinhardtii itself. A multiple sequence alignment of the C. reinhardtii LCIB, LCIC, LCID, and LCIE proteins is shown in FIG. 2. The function(s) of these genes are not currently known, although they are all believed to be localized to the chloroplast, as predicted by ChloroP, a neural network-based method for predicting chloroplast transit peptides and their cleavage sites (Emanuelsson et al., 1999, Protein Sci. 8(5):978-84). LCIE appears to be soluble, as the Phobius tool (http://www.ebi.ac.uk/Tools/pfa/phobius/) predicts no transmembrane domains. Neither the NCBI (https://www.ncbi.nlm.nih.gov/Structure/cdd/wrpsb.cgi) nor the KEGG (www.genome.jp/tools/motif/) web-based motif searches identify any recognizable conserved protein domains, but the ProSite motif search tool (http://prosite.expasy.org/) predicts a putative AMP-binding domain spanning residues 57-68. Because CCP1 appears to be a mitochondrial transporter, it could affect not only the metabolism of the mitochondrion but also the metabolite profile of other compartments of the cell, such as the chloroplast. The fact that CCP1-like and LCID/E genes are found together in many organisms could indicate that they act in concert. For example, LCID and LCIE may alter chloroplast metabolism in a manner amenable to CCP1 function in the mitochondrion. Because of the high degree of similarity among LCIB, LCIC, LCID, and LCIE, a yield advantage could be derived by combining in a crop species the expression of C. reinhardtii CCP1 or CCP2 with expression of LCIE and/or LCID genes from C. reinhardtii.

### Example 2. Orthologs of C. Reinhardtii LCID/E from Eukaryotic Algae

A similar advantage could also be obtained by co-expressing in a crop species CCP1 orthologs and LCID/E orthologs from other species. Gene or protein orthologs of the C. reinhardtii LCID/E protein were identified using results derived from a BLASTP 2.6.0+ search (Altschul et al., 1997, Nucleic Acids Res. 25:3389-3402) using C. reinhardtii LCIE as the query sequence. TABLE 3 provides a listing of the proteins that have E values of less than le-45, excluding those of C. reinhardtii. This serves as an example and not as an exhaustive list. Those skilled in the art will find many more such examples in the course of homology searches. A common feature of these genes is that they all encode proteins having chloroplast targeting signal as predicted by ChloroP, as discussed above.

TABLE 3
Protein orthologs of C. reinhardtii LCIE protein that have E values
of less than 1e−45 (excluding those of C. reinhardtii).

| GenBank | Protein | E |
|---|---|---|
| Accession | [Organism] | value |
| KXZ50484.1 | hypothetical protein GPECTOR_16g659 | 0.0 |
| | [Gonium pectorale] | |
| KXZ50473.1 | hypothetical protein GPECTOR_16g647 | 0.0 |
| | [Gonium pectorale] | |
| KXZ50485.1 | hypothetical protein GPECTOR_16g660 | 0.0 |
| | [Gonium pectorale] | |
| XP_002954408.1 | low-CO2 inducible protein | 0.0 |
| | [Volvox carted f. nagariensis] | |
| ABM47323.1 | low-CO2 inducible protein | 1e−168 |
| | [Volvox carted f. nagariensis] | |
| GEEU01047164.1 | Ettlia oleoabundans 8472_c0_seq1 | 2e−141 |
| | transcribed RNA sequence | |
| GAPD01006726.1 | Chlorella sorokiniana comp7492_c0_seq1 | 9e−134 |
| | transcribed RNA sequence | |
| XP_002951506.1 | GON30 protein | 5e−105 |
| | [Volvox carti f. nagariensis] | |
| KXZ54572.1 | hypothetical protein GPECTOR_4g637 | 4e−105 |
| | [Gonium pectorale] | |
| XP_002957342.1 | hypothetical protein VOLCADRAFT_84075 | 3e−104 |
| | [Volvox carteri f. nagariensis] | |
| KXZ44952.1 | hypothetical protein GPECTOR_60g729 | 5e−103 |
| | [Gonium pectorale] | |
| XP_005845813.1 | hypothetical protein CHLNCDRAFT_58491 | 4e−91 |
| | [Chlorella variabilis] | |
| XP_005845923.1 | hypothetical protein CHLNCDRAFT_53648 | 2e−89 |
| | [Chlorella variabilis] | |
| XP_005845814.1 | hypothetical protein CHLNCDRAFT_8957 | 6e−86 |
| | [Chlorella variabilis] | |
| XP_011401950.1 | hypothetical protein F751_4065 | 3e-85 |
| | [Auxenochlorella protothecoides] | |
| XP_005647048.1 | hypothetical protein COCSUDRAFT_16853 | 1e−83 |
| | [Coccomyxa subellipsoidea C-169] | |
| XP_005848573.1 | hypothetical protein CHLNCDRAFT_145145 | 8e−83 |
| | [Chlorella variabilis] | |
| XP_005846604.1 | hypothetical protein CHLNCDRAFT8980 | 1e−81 |
| | [Chlorella variabilis] | |
| XP_013893257.1 | low-CO2-inducible protein | 5e−69 |
| | [Monoraphidiurn neglectum] | |
| XP_005851773.1 | hypothetical protein CHLNCDRAFT_33527 | 6e−69 |
| | [Chlorella variabilis] | |
| KDD75410.1 | hypothetical protein H632_c697p1 | 2e−66 |
| | [Helicosporidium sp. ATCC 50920] | |
| XP_013893360.1 | low-co2 inducible protein lcib | 2e−63 |
| | [Monoraphidium neglectum] | |
| XP_001419219.1 | Conserved hypothetical protein | 6e−51 |
| | [Ostreococcus lucimarinus CCE9901] | |
| XP_007515175.1 | predicted protein | 6e−49 |
| | [Bathycoccus prasinos] | |
| CEG01597.1 | unnamed product | 3e−46 |
| | [Ostreococcus tauri] | |

### Example 3. Algal and Plant CCP1 Mitochondrial Transporter Genes and Orthologs for Use in Combination with the LCID/E Genes and Orthologs

Eukaryotic algal and plant mitochondrial transporter genes useful for increasing photosynthesis, reducing transpiration rates, and/or increasing biomass and/or seed yield have been identified, as discussed above. For context, U.S. Provisional Appl. No. 62/520,785 describes that plant CCP1-like mitochondrial transporter proteins appear to cluster into two distinct groups, termed Tier 1 CCP1 orthologs and Tier 2 CCP1 orthologs, based on similarities of predicted amino acid sequence and structure with respect to CCP1 of Chlamydomonas reinhardtii. The plant Tier 1 CCP1 orthologs exhibit about 60% sequence identity with respect to CCP1 of Chlamydomonas reinhardtii, cluster narrowly based on the degree of their sequence similarity, and have been identified thus far only in four plant species, Zea nicaraguensis, Erigeron breviscapus, Cosmos bipinnatus, and Poa pratensis, none of which are particularly closely related phylogenetically. The plant Tier 2 CCP1 orthologs exhibit about 30% sequence identity with respect to CCP1 of Chlamydomonas reinhardtii, substantially lower than for Tier 1, also cluster narrowly based on the degree of their sequence similarity, and would appear to be more common, having been identified thus far in six major crop species, Zea mays (also termed maize), Triticum aestivum, Solanum tuberosum, Glycine max, Oryza sativa, and Sorghum bicolor. This was surprising because, among other reasons, Zea nicaraguensis, again teosinte, is a wild progenitor of Zea mays, again maize, and thus the two are closely related phylogenetically, yet Zea nicaraguensis includes a Tier 1 CCP1, whereas Zea mays includes a Tier 2 CCP1.

In general, the algal and plant CCP1 mitochondrial transporter genes useful for co-expression with an LCID/E protein should encode proteins that have the specific structural motifs, and should be localized to the mitochondrial membrane. Lists of useful genes encoding such algal and plant CCP1 mitochondrial transporter proteins, and the corresponding structural motifs, are shown in TABLE 4, TABLE 5, and TABLE 6.

TABLE 4
Proteins of eukaryotic algae with homology to Chlamydomonas reinhardtii CCP1.

| | Nucleotide | | | | | | | |
|---|---|---|---|---|---|---|---|---|
| | Accession | Number | Homology to CCP1 | | | | | |
| | (and SEQ ID NO | of | Consensus | Identity | | | | |
| | of corresponding | amino | positions | Positions | Program | | | |
| Organism | protein) | acids | (%) | (%) | Motif Finder^{b} | | ProSite^{c} | |
| Chlamydomonas | XM_001692145.1 | 358 | 100 | 100 | Mitochondrial | 3 predicted | Solute | 3 predicted |
| reinhardtii | (SEQ ID NO: 9) | | | | carrier protein | motifs spanning | carrier | motifs spanning |
| | | | | | | amino acids 28- | protein^{d} | amino acids 22- |
| | | | | | | 119; 129-235; | | 118; 131-231; |
| | | | | | | & 245-334 | | & 246-333 |
| Gonium | KXZ50472.1 | 356 | 93.9 | 84.4 | Mitochondrial | 3 predicted | Solute | 3 predicted |
| pectorale | (SEQ ID NO: 44) | | | | carrier protein | motifs spanning | carrier | motifs spanning |
| | | | | | | amino acids 27- | protein^{d} | amino acids 22- |
| | | | | | | 119; 129-234; | | 118; 128-230; |
| | | | | | | & 244-333 | | & 245-332 |
| Gonium | KXZ50486.1 | 354 | 91.4 | 83.9 | Mitochondrial | 3 predicted | Solute | 3 predicted |
| pectorale | (SEQ ID NO: 45) | | | | carrier protein | motifs spanning | carrier | motifs spanning |
| | | | | | | amino acids 27- | protein^{d} | amino acids 22- |
| | | | | | | 119; 129-234; | | 118; 128-230; |
| | | | | | | & 244-333 | | & 245-332 |
| Volvox carteri | XP_002951243.1 | 339 | 89.7 | 80.4 | Mitochondrial | 3 predicted | Solute | 3 predicted |
| f. nagariensis | (SEQ ID NO: 46) | | | | carrier protein | motifs spanning | carrier | motifs spanning |
| | | | | | | amino acids 21- | protein^{d} | amino acids 15- |
| | | | | | | 112, 122-215, | | 111, 121-212, |
| | | | | | | 227-315 | | 227-314 |
| Volvox carteri | XM_002951197.1 | 339 | 89.7 | 80.4 | Mitochondrial | 3 predicted | Solute | 3 predicted |
| | (SEQ ID NO: 47) | | | | carrier protein | motifs spanning | carrier | motifs spanning |
| | | | | | | amino acids 21- | protein^{d} | amino acids 15- |
| | | | | | | 112; 122-215; | | 111; 121-212; |
| | | | | | | & 227-315 | | & 227-314 |
| Ettlia | GEEU01047164.1 | 353^{a} | 76.0 | 61.5 | Mitochondrial | 3 predicted | Solute | 3 predicted |
| oleoabundans | (SEQ ID NO: 48) | | | | carrier protein | motifs spanning | carrier | motifs spanning |
| | | | | | | amino acids 28- | protein^{d} | amino acids 22- |
| | | | | | | 119, 128-233, 243- | | 118, 131-231, 242- |
| | | | | | | 331 | | 329 |
| Chlorella | GAPD01006726.1 | 354^{a} | 74.7 | 62.6 | Mitochondrial | 3 predicted | Solute | 3 predicted |
| sorokiniana | (SEQ ID NO: 49) | | | | carrier protein | motifs spanning | carrier | motifs spanning |
| | | | | | | amino acids 25- | protein^{d} | amino acids 20- |
| | | | | | | 117; 128-228; | | 116; 131-227; |
| | | | | | | & 243-329 | | & 238-325 |
| Chlorella | XM_005846489.1 | 303 | 40.9 | 26.9 | Mitochondrial | 3 predicted | Solute | 3 predicted |
| variabilis | (SEQ ID NO: 50) | | | | carrier protein | motifs spanning | carrier | motifs spanning |
| | | | | | | amino acids 4- | protein^{d} | amino acids 3- |
| | | | | | | 88; 97-199; & | | 86; 96-200; & |
| | | | | | | 212-301 | | 212-300 |
| Chlorella | XM_005852157.1 | 323 | 40.2 | 26.5 | Mitochondrial | 3 predicted | Solute | 3 predicted |
| variabilis | (SEQ ID NO: 51) | | | | carrier protein | motifs spanning | carrier | motifs spanning |
| | | | | | | amino acids 26- | protein^{d} | amino acids 25- |
| | | | | | | 115; 125-221; | | 112; 124-218; |
| | | | | | | & 229-322 | | & 230-319 |
| Chlorella | XM_005843001.1 | 323 | 42.7 | 27.2 | Mitochondrial | 3 predicted | Solute | 3 predicted |
| variabilis | (SEQ ID NO: 52) | | | | carrier protein | motifs spanning | carrier | motifs spanning |
| | | | | | | amino acids 9- | protein^{d} | amino acids 8- |
| | | | | | | 90; 108-187; & | | 92; 101-189; & |
| | | | | | | 225-307 | | 221-308 |
| Chondrus | XM_005712871.1 | 328 | 39.1 | 22.0 | Mitochondrial | 3 predicted | Solute | 3 predicted |
| crispus | (SEQ ID NO: 53) | | | | carrier protein | motifs spanning | carrier | motifs spanning |
| | | | | | | amino acids 40- | protein^{d} | amino acids 39- |
| | | | | | | 127; 137-230; | | 128;135-227; & |
| | | | | | | & 239-326 | | 238-325 |
| Chlorella | XM_005851446.1 | 306 | 38.9 | 25.4 | Mitochondrial | 3 predicted | Solute | 3 predicted |
| variabihs | (SEQ ID NO: 54) | | | | carrier protein | motifs spanning | carrier | motifs spanning |
| | | | | | | amino acids 11- | protein^{d} | amino acids 11- |
| | | | | | | 101; 112-206; | | 100; 112-203; |
| | | | | | | & 213-299 | | & 212-298 |
| Chondrus | XM_005715654.1 | 233 | 35.7 | 24.5 | Mitochondrial | 3 predicted | Solute | 3 predicted |
| crispus | (SEQ ID NO: 55) | | | | carrier protein | motifs spanning | carrier | motifs spanning |
| | | | | | | amino acids 3- | protein^{d} | amino acids 1- |
| | | | | | | 40; 47-132; & | | 37; 47-131; & |
| | | | | | | 141-231 | | 142-229 |
| Chondrus | XM_005713259.1 | 194 | 31.7 | 20.6 | Mitochondrial | 2 predicted | Solute | 2 predicted |
| crispus | (SEQ ID NO: 56) | | | | carrier protein | motifs spanning | carrier | motifs spanning |
| | | | | | | amino acids 7- | protein^{d} | amino acids 8- |
| | | | | | | 93 & 102-191 | | 92 & 103-190 |

^{a}sequence from first methionine of deposited transcribed mRNA sequence to first stop codon
^{b}http://www.genome.jp/tools/motif/
^{c}http://prosite.expasy.org/
^{d}predicted as one of several substrate carrier proteins involved in energy transfer in the inner mitochondrial membrane (http://prosite.expasy.org/cgi-bin/prosite/nicedoc.pl?PS50920)

TABLE 5
CCP1 of Chlamydomonas reinhardtii and “Tier 1” orthologs from Ettlia oleoabundans and various land plants.

| | | | | | | Program | |
|---|---|---|---|---|---|---|---|
| | | | | Homology to CCP1 | | | ProSite^{c} |
| | | | Number | Consensus | Identity | Motif Finder^{b} | SOLCAR domains |
| | | | of Amino | Positions | Positions | Mito_carr domains predicted | predicted |
| Organism | Type | GenBank Accession | Acids | (%) | (%) | (residues) | (residues) |
| Chlamydomonas | Algae | XM_001692145.1 | 358 | 100 | 100 | 28-119, 129-235, 245-334 | 22-118, 131-231, 246-333 |
| reinhardtii | | (SEQ ID NO: 9) | | | | | |
| Ettlia | Algae | GEEU01047164.1 | 353^{a} | 76.0 | 61.5 | 28-119, 128-233, 243-331 | 22-118, 131-231, 242-329 |
| oleoabundans | | (SEQ ID NO: 48) | | | | | |
| Erigeron | Land | GDQF01162509.1 | 352^{a} | 74.9 | 62.6 | 28-120, 128-233, 242-331 | 22-118, 128-231, 242-329 |
| breviscapus | plants | (SEQ ID NO: 57) | | | | | |
| Zea | Land | GBZQ01039302.1 | 354^{a} | 74.9 | 62.1 | 29-121, 129-233, 241-331 | 23-119, 132-231, 242-329 |
| nicaraguensis | plants | (SEQ ID NO: 58) | | | | | |
| Poa pratensis | Land | GEBH01135677.1 | 141^{d} | 26.3 | 22.6 | 5-51, 59-139 | 1-48, 60-141 |
| | plants | (SEQ ID NO: 59) | | | | | |
| Cosmos | Land | GEZQ01046902.1 | 354 | 76.3 | 63.0 | 29-121, 130-233, 241-331 | 23-119, 132-231, 242-329 |
| bipinnatus | plants | (SEQ ID NO: 60) | | | | | |

^{a}Sequence from first methionine of deposited transcribed mRNA sequence to first stop codon.
^{b}Website: genome.jp/tools/motif
^{c}Website: prosite.expasy.org
^{d}Partial protein sequence

TABLE 6
CCP1 of Chlamydomonas reinhardtii and CCP1 orthologs
from land plants (Tier 2) corresponding to major crops.

| | | Number | Homology to CCP1 | |
|---|---|---|---|---|
| | | of | Consensus | Identity |
| | GenBank | Amino | Positions | Positions |
| Organism | Accession | Acids | (%) | (%) |
| Chlamydomonas | XM_001692145.1 | 358 | 100 | 100 |
| reinhardtii | (SEQ ID NO: 9) | | | |
| Glycine max | KRH74426.1 | 297 | 45.9 | 30.9 |
| | (SEQ ID NO: 61) | | | |
| Zea mays | NP_001141073.1 | 296 | 46.9 | 31.2 |
| | (SEQ ID NO: 62) | | | |
| Oryza sativa | XP_015614184.1 | 296 | 47.1 | 30.2 |
| Japonica Group | (SEQ ID NO: 63) | | | |
| Triticum | CDM80555.1 | 324 | 43.1 | 27.5 |
| aestivum | (SEQ ID NO: 64) | | | |
| Sorghum | XP_002464891.1 | 296 | 47.5 | 31.1 |
| bicolor | (SEQ ID NO: 65) | | | |
| Solanum | XP_006361187.1 | 323 | 48.3 | 32.6 |
| tuberosum | (SEQ ID NO: 66) | | | |

### Example 4. Expression of LCID/E Genes in Camelina and Canola

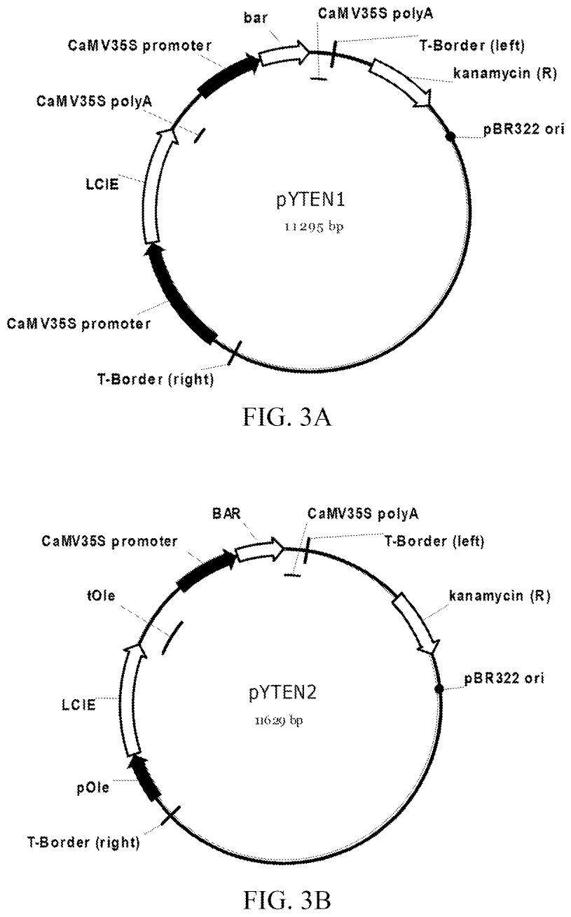

In order to express an LCID/E gene in a crop species, it can be placed downstream of a suitable promoter and upstream of a suitable terminator sequence within a transformable plant vector. Vectors suitable for transformation of Camelina and canola are shown in FIG. 3A-B, and include pYTEN1 (SEQ ID NO: 67), containing the LCIE gene flanked by a constitutive CaMV35S promoter and terminator from the cauliflower mosaic virus, and pYTEN2 (SEQ ID NO: 68), containing the LCIE gene flanked by the seed specific promoter and terminator from the soya bean oleosin isoform A gene (Glyma.16G071800, TABLE 1, above). Both vectors also contain an expression cassette for the bar gene, driven by the CaMV35S promoter, which allows selection of transgenic plants by providing them resistance to the herbicide bialophos. Additional promoters useful for expressing LCIE in dicot crops such as canola or camelina are listed in TABLE 1. It will be apparent to those skilled in the art that a wide range of promoters are available for dicots that can selected based on their tissue specificity for expression of LCIE. The list of promoters in TABLE 1 is not exhaustive and it will be apparent to those skilled in the art that other constitutive promoters, as well as tissue specific promoters, including seed specific and leaf or green biomass specific promoters, can be used.

Camelina is transformed as follows.

In preparation for plant transformation experiments, seeds of Camelina sativa germplasm 10CS0043 (abbreviated WT43, obtained from Agriculture and Agri-Food Canada) are sown directly into 4 inch (10 cm) pots filled with soil in the greenhouse. Growth conditions are maintained at 24° C. during the day and 18° C. during the night. Plants are grown until flowering. Plants with a number of unopened flower buds are used in ‘floral dip’ transformations.

Agrobacterium strain GV3101 (pMP90) is transformed with either pYTEN1 or pYTEN2 using electroporation. A single colony of GV3101 (pMP90) containing the construct of interest is obtained from a freshly streaked plate and is inoculated into 5 mL LB medium. After overnight growth at 28° C., 2 mL of culture is transferred to a 500-mL flask containing 300 mL of LB and incubated overnight at 28 ° C. Cells are pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants are transformed by “floral dip” using the pYTEN1 or pYTEN2 transformation constructs as follows. Pots containing plants at the flowering stage are placed inside a 460 mm height vacuum desiccator (Bel-Art, Pequannock, N.J., USA). Inflorescences are immersed into the Agrobacterium inoculum contained in a 500-ml beaker. A vacuum (85 kPa) is applied and held for 5 min. Plants are removed from the desiccator and are covered with plastic bags in the dark for 24 h at room temperature. Plants are removed from the bags and returned to normal growth conditions within the greenhouse for seed formation (Ti generation of seed).

T1 seeds are planted in soil and transgenic plants are selected by spraying a solution of 400 mg/L of the herbicide Liberty (active ingredient 15% glufosinate-ammonium). This allows identification of transgenic plants containing the bar gene on the T-DNA in the plasmid vectors pYTEN1 and pYTEN2 (FIG. 3). Transgenic plant lines are further confirmed using PCR with primers specific to the gene of interest. PCR positive lines are grown in a greenhouse to produce the next generation of seed (T2 seed). Seeds are isolated from each plant and are dried in an oven with mechanical convection set at 22° C. for two days. The weight of the entire harvested seed obtained from individual plants is measured and recorded.

Canola is transformed as follows.

In preparation for plant transformation experiments, seeds of Brassica napus cv DH12075 (obtained from Agriculture and Agri-Food Canada) are surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution is decanted and 0.025% mercuric chloride with a drop of Tween 20 is added and the seeds are sterilized for another 10 minutes. The seeds are then rinsed three times with sterile distilled water. The sterilized seeds are plated on half strength hormone-free Murashige and Skoog (MS) media (Murashige T, Skoog F (1962). Physiol Plant 15:473-498) with 1% sucrose in 15×60 mm petri dishes that are then placed, with the lid removed, into a larger sterile vessel (Majenta GA7 jars). The cultures are kept at 25° C., with 16 h light/8 h dark, under approx. 70-80 μE of light intensity in a tissue culture cabinet. Seedlings that are 4-5 days old are used to excise fully unfolded cotyledons along with a small segment of the hypocotyl. Excisions are made so as to ensure that no part of the apical meristem is included.

The Agrobacterium strain GV3101 (pMP90) carrying pYTEN1 or pYTEN2 (FIG. 3A-B) transformation vectors are grown overnight in 5 ml of LB media with 50 mg/L kanamycin, gentamycin, and rifampicin. The culture is centrifuged at 2000 g for 10 min, the supernatant is discarded and the pellet is suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons are collected in Petri dishes with ˜1 ml of sterile water to keep them from wilting. The water is removed prior to inoculation and explants are inoculated in mixture of 1 part Agrobacterium suspension and 9 parts inoculation medium in a final volume sufficient to bathe the explants. After explants are well exposed to the Agrobacterium solution and inoculated, a pipet is used to remove any extra liquid from the petri dishes.

The Petri plates containing the explants incubated in the inoculation media are sealed and kept in the dark in a tissue culture cabinet set at 25° C. After 2 days the cultures are transferred to 4° C. and incubated in the dark for 3 days. The cotyledons, in batches of 10, are then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures are kept in a tissue culture cabinet set at 25° C., 16 h/8 h, with a light intensity of about 125 μmol m⁻² s⁻¹. The cotyledons are transferred to fresh selection every 3 weeks until shoots are obtained. The shoots are excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that formed at the base of shoots with normal morphology is cut off and shoots are transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots are hardened and transferred to 6″ pots in the greenhouse to collect T1 transgenic seeds. T1 seed is grown in a greenhouse to produce T2 seed. The mass of the total seed per plant is collected to compare seed yield of transgenics to wild-type control plants.

### Example 5. Expression of LCID/E Genes in Soybean

For transformation of soybean, a biolistic method is employed. The transformation, selection, and plant regeneration protocol for soybean is adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and requires an expression cassette for the LCIE gene, containing a suitable promoter, the LCIE gene, and a suitable polyadenylation sequence, and an expression cassette for a selectable marker, such as the hygromycin resistance marker. The LCIE and hygromycin resistance cassette can be co-localized on one plasmid or isolated DNA fragment, or alternatively, two separate plasmids or isolated DNA fragments containing the expression cassettes can be co-bombarded.

Vectors pYTEN1 and pYTEN2 (FIG. 3) can be optimized for transformation into soybean by replacing the bar expression cassette with an expression cassette encoding the hygromycin gene. A DNA fragment(s) containing the CCP1, LCIE, and hygromycin resistance gene expression cassettes can be excised and introduced into soybean using the biolistics method described below. In some cases, it may be desirable to optimize the promoter's expression of CCP1 and LCIE. Promoters for expression of these transgenes can be selected from those listed in TABLE 1, depending on the desired tissue specificity for expression, or any other promoter that has provide goods expression in dicots.

For soybean transformation, the purified DNA fragment(s) are introduced into embryogenic cultures of soybean Glycine max cultivars X5 and Westag97 via biolistics, to obtain transgenic plants. The transformation, selection, and plant regeneration of soybean is performed as follows.

Induction and Maintenance of Proliferative Embryogenic Cultures: Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis is excised and explants are cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/1 2,4-D, pH 5.7]. The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/12,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1 005 p.m pore size, Band SH Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/∥l of insert DNA (of pYTEN1 or pYTEN2) and 2.5 μl of 100 ng/μl selectable marker DNA (cassette for hygromycin selection) are added to 3 mg gold particles suspended in 50 μl sterile dH₂0 and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA is resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection: The bombarded tissue is cultured on embryo proliferation medium described above for 12 days prior to subculture to selection medium (embryo proliferation medium containing 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approx. 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant regeneration: Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/1 MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, Wash., USA), and covered with a transparent plastic lid to maintain high humidity. The flats are placed in a controlled growth cabinet at 26/24° C. (day/night), 18 h photoperiod at a light intensity of 150 μmol m⁻² s⁻¹. At the 2-3 trifoliate stage (2-3 weeks), the plantlets with strong roots are transplanted to pots containing a 3:1:1:1 mix of ASB Original Grower Mix (a peat-based mix from Greenworld, ON, Canada):soil: sand: perlite and grown at 18-h photoperiod at a light intensity of 300-400 μmolm⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

There are also Agrobacterium-mediated transformation methods for soybean that can be used to generate similar transgenic plants expressing the LCIE gene.

### Example 6. Expression of LCID/E Genes in Rice and Maize

For transformation of rice, a binary vector containing an expression cassette with a promoter, the LCIE gene, and a polyadenylation sequence, as well as an expression cassette for a selectable marker, such as the hygromycin resistance marker, is prepared. The LCIE and hygromycin resistance cassette can be co-localized on one binary vector or, alternatively, positioned on two separate binary vectors that can be co-bombarded.

Several promoters were chosen for expression of the LCIE gene in rice based on their experimental or in silico predicted expression profiles in rice seed and are shown in TABLE 2, above. It will be apparent to those skilled in the art that TABLE 2 is not an exhaustive list of promoters that can be used for expression in rice and that there are many additional promoters that would work to practice the invention, depending on the tissue specificity desired for expression of the transgene. The promoter from the rice ADP-glucose pyrophosphorylase (AGPase) gene (GenBank: AY427566.1, LOC_Os01g44220) was chosen since it has been shown to be expressed in the seed as well as well as the phloem of vegetative tissues in rice (Qu, L. Q. and Takaiwa, F., 2004, Plant Biotechnology Journal, 2, 113-125). The promoter from the rice glutelin C (GluC) gene (GenBank: EU264107.1, LOC_Os02g25640) has been shown to be expressed in the whole endosperm of rice seed (Qu, L. Q. et al., 2008, Journal of Experimental Biology, 59, 2417-2424). The promoter from the rice beta-fructofuranosidase insoluble isoenzyme 1 (CIN1) gene was chosen based on in silico expression data showing expression throughout various developmental stages but with highest expression in the inflorescence and seeds (Rice Genome Annotation Project; http://rice.plantbiology.msu.edu/cgi-bin/ORF_infopage.cgi?orf=LOC_Os02g33110.1).

In preparation for rice transformation, callus of the rice cultivar Nipponbare is initiated from mature, dehusked, surface sterilized seeds on N6-basal salt callus induction media (N6-CI; contains per liter 3.9 g CHU (N₆) basal salt mix [Sigma Catalog #C1416]; 10 ml of 100× N6-vitamins [contains in final volume of 500 mL, 100 mg glycine, 25 mg nicotinic acid, 25 mg pyridoxine hydrochloride and 50 mg thiamin hydrochloride]; 0.1 g myo-inositol; 0.3 g casamino acid (casein hydrolysate); 2.88 g proline; 10 ml of 100× 2,4-dichlorophenoxyacetic acid (2,4-D), 30 g sucrose, pH 5.8 with 4 g gelrite or phytagel). Approximately 100 seeds are used for each transformation. The frequency of callus induction is scored after 21 days of culture in the dark at 27±1° C. Callus induction from the scutellum with a high frequency (of about 96% total callus induction) is observed.

Rice transformation vectors are transformed into Agrobacterium strain AGL1. Agrobacterium containing a vector is resuspended in 10 mL of MG/L medium (5 g tryptone, 2.4 g yeast extract, 5 g mannitol, 5 g Mg₂SO₄, 0.25 g K₂HPO₄, 1 g glutamic acid and 1 g NaCl) to a final OD600 of 0.3. Approximately twenty-one day old scutellar embryogenic callus are cut to about 2-3 mm in size and are infected with Agrobacterium containing the transformation vector for 5 min. After infection, the calli are blotted dry on sterile filter papers and transferred onto co-cultivation media (N6-CC; contains per liter 3.9 g CHU (N₆) basal salt mix; 10 ml of 100× N6-vitamins; 0.1 g myo-inositol; 0.3 g casamino acid; 10 ml of 100× 2,4-D, 30 g sucrose, 10 g glucose, pH 5.2 with 4 g gelrite or phytagel and 1 mL of acetosyringone [19.6 mg/mL stock]). Co-cultivated calli are incubated in the dark for 3 days at 25° C. After three days of co-cultivation, the calli are washed thoroughly in sterile distilled water to remove the bacteria. A final wash with a timentin solution (250 mg/L) is performed and calli are blotted dry on sterile filter paper. Callus are transferred to selection media (N6-SH; contains per liter 3.9 g CHU (N₆) basal salt mix, 10 ml of 100×N6-vitamins, 0.1 g myo-inositol, 0.3 g casamino acid, 2.88 g proline, 10 ml of 100×, 2,4-D, 30 g sucrose, pH 5.8 with 4 g phytagel and 500 μL of hygromycin (stock concentration: 100 mg/ml) and incubated in the dark for two-weeks at 27±1° C. The transformed calli that survived the selection pressure and that proliferated on N6-SH medium are sub-cultured on the same media for a second round of selection. These calli are maintained under the same growth conditions for another two-weeks. The number of plants regenerated after 30 days on N6-SH medium is scored and the frequency calculated. After 30 days, the proliferating calli are transferred to regeneration media (N6-RH medium; contains per liter 4.6 g MS salt mixture, 10 ml of 100× MS-vitamins [MS-vitamins contains in 500 mL final volume 250 mg nicotinic acid, 500 mg pyridoxine hydrochloride, 500 mg thiamine hydrochloride, 100 mg glycine], 0.1 g myo-inositol, 2 g casein hydrolysate, 1 ml of 1,000×1-naphtylacetic acid solution [NAA; contains in 200 mL final volume 40 mg NAA and 3 mL of 0.1 N NaOH], 20 ml of 50× kinetin [contains in 500 mL final volume 50 mg kinetin and 20 mL 0.1 N HCl], 30 g sucrose, 30 g sorbitol, pH 5.8 with 4 g phytagel and 500 μl of a 100 mg/mL hygromycin stock). The regeneration of plantlets from these calli occurs after about 4-6 weeks. Rooted plants are transferred into peat-pellets for one week to allow for hardening of the roots. The plants are then kept in zip-loc bags for acclimatization. Plants are transferred into pots and grown in a greenhouse to maturity prior to seed harvest (T1 generation). T1 seed is grown in a greenhouse to produce T2 seed. The mass of the total seed per plant is collected to compare seed yield of transgenics to wild-type control plants.

For transformation of maize, a binary vector containing a promoter, the LCIE gene, and a terminator is constructed and an expression cassette for a selectable marker, such as the bar gene imparting resistance to the herbicide bialophos, are included. In preparation for transformation, the binary vector is transformed into an Agrobacterium tumefaciens strain, such as A. tumefaciens strain EHA101. Agrobacterium-mediated transformation of maize can be performed following a previously described procedure (Frame et al., 2006, Agrobacterium Protocols Wang K., ed., Vol. 1, pp 185-199, Humana Press) as follows.

Plant Material: Plants grown in a greenhouse are used as an explant source. Ears are harvested 9-13 dafter pollination and surface sterilized with 80% ethanol.

Explant Isolation, Infection and Co-Cultivation: Immature zygotic embryos (1.2-2.0 mm) are aseptically dissected from individual kernels and incubated in A. tumefaciens strain EHA101 culture (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300mg/1 cysteine, 5 μM silver nitrate and 100 μM acetosyringone) and incubated at 20° C., in the dark for 3 d. Embryos are transferred to N6 resting medium containing 100 mg/1 cefotaxime, 100 mg/l vancomycin and 5 μM silver nitrate and incubated at 28° C., in the dark for 7 d.

Callus Selection:

All embryos are transferred on to the first selection medium (the resting medium described above supplemented with 1.5 mg/l bialaphos) and incubated at 28° C., in the dark for 2 weeks followed by subculture on a selection medium containing 3 mg/l bialaphos. Proliferating pieces of callus are propagated and maintained by subculture on the same medium every 2 weeks.

Plant Regeneration and Selection:

Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C., in the dark for 2 to 3 weeks. Mature embryos formed during this period are transferred on to regeneration medium II (the same as regeneration medium I with 3 mg/l bialaphos) for germination in the light (25° C., 80-100 μE/m²/s light intensity, 16/8-h photoperiod). Regenerated plants are ready for transfer to soil within 10-14 days. Plants are grown in a greenhouse to produce T1 seed. T1 seed is grown in soil in a greenhouse to produce T2 seed. The mass of the total seed per plant is collected to compare seed yield of transgenics to wild-type control plants.

### Example 7. Expression of LCID/E Genes in Plants in Combination with CCP1 Genes

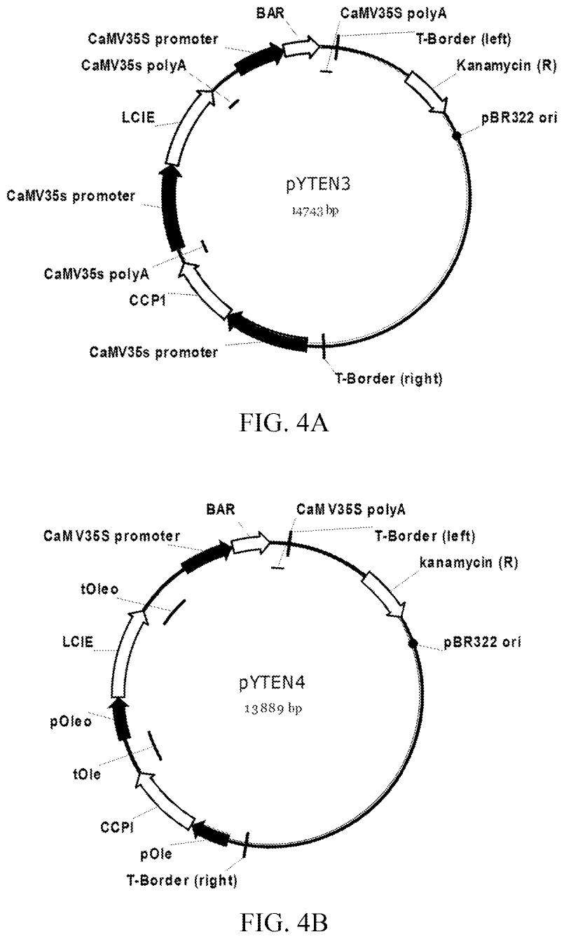

An LCID/E gene can be co-expressed in a plant with a CCP1 gene by placing expression cassettes for the LCID/E gene and the CCP1 gene on the same transformation vector and transforming the plant. It will be apparent to those skilled in the art that the gene cassettes can contain a variety of different promoters to control expression, including seed specific promoters, constitutive promoters, leaf specific promoters, or other tissue specific promoters. Two examples given here are pYTEN3 (FIG. 4A, SEQ ID 69), in which the expression of both genes is controlled by constitutive CaMV35S promoter and terminators, and pYTEN4 (FIG. 4B, SEQ ID 70), in which the expression of both genes is controlled by seed-specific oleosin promoters and matching oleosin terminators. Vectors pYTEN3 and pYTEN4 are designed for transformation into dicots, including Camelina and canola, whose transformation procedures are described above. Co-transformation of pYTEN3 and pYTEN4 into the same plant may provide enhanced yield benefits.

It will be apparent to those skilled in the art that co-expression of LCID/E and CCP1 genes can also be achieved by co-transformation of separate vectors that contain an LCID/E expression cassette on one plasmid and a CCP1 expression cassette on another plasmid and screening the transformants for the presence of both expression cassettes. It will also be apparent to those skilled in the art that co-expression of LCID/E and CCP1 genes can be achieved by crossing plants expressing the individual genes to obtain a plant expressing both genes.

Vectors pYTEN3 and pYTEN4 can be optimized for transformation into soybean by replacing the bar expression cassette with an expression cassette encoding the hygromycin gene. A DNA fragment(s) containing the CCP1, LCIE, and hygromycin resistance gene expression cassettes can be excised and introduced into soybean using the biolistics method described above. In some cases, it may be desirable to optimize the promoter's expression of CCP1 and LCIE. Promoters for expression of these transgenes can be selected from those listed in Table 1, depending on the desired tissue specificity for expression, or any other promoter that has provide goods expression in dicots.

Vectors pYTEN3 and pYTEN4 can be optimized for transformation into rice by replacing the bar expression cassette with an expression cassette encoding the hygromycin gene. In some instances, it may be desirable to optimize the promoters driving the expression of the LCIE and CCP1 genes using a monocot specific promoter, such as the ones described in TABLE 2, above, or any other promoter that provides good expression in monocots. The choice of the promoter may be dictated by the desired tissue specificity for expression. The modified binary vectors are introduced into an Agrobacterium strain, such as Agrobacterium strain AGL1, and the rice transformation procedure described above is followed.

Vectors pYTEN3 and pYTEN4 can be optimized for transformation into maize by using a monocot specific promoter, such as the ones described in TABLE 2, or any other promoter that provides good expression in monocots, to drive the expression of the LCIE and CCP1 genes. The choice of the promoter may be dictated by the desired tissue specificity for expression. The modified binary vectors are introduced into an Agrobacterium strain, such as A. tumefaciens strain EHA101, and the maize transformation procedure described above is followed.

### Example 8. Identification of LCD and LCIE Genes and Orthologs of Land Plants

Standard BLAST searches using C. reinhardtii LCIE as a query sequence, focused on “green plants,” and excluding “green algae,” reveal that certain land plants encode orthologs of Chlamydomonas reinhardtii LCID/E. As discussed above, this was surprising because, among other reasons, it is not apparent whether or to what extent LCID and/or LCIE may play roles in carbon-concentrating mechanisms to increase intracellular concentrations of dissolved inorganic carbon. This also was surprising because initial results suggest that only a small number of species of land plants encode the LCID/E orthologs, the various species of land plants that encode the LCID/E orthologs appear to be phylogenetically distant from each other, not closely related, and yet the LCID/E orthologs encoded by each of the various species of land plants appear to be highly similar to LCID and LCIE of a particular algal species, Ettlia oleoabundans.

As discussed above, U.S. Provisional Appl. No. 62/520,785 describes that plant CCP1-like mitochondrial transporter proteins appear to cluster into two distinct groups, termed Tier 1 CCP1 orthologs and Tier 2 CCP1 orthologs. U.S. Provisional Appl. No. 62/520,785 also describes that certain algal and plant CCP1 orthologs, termed “Tier 1B” CCP1 orthologs, seem to be more closely related to each other than to the other algal CCP1 orthologs, termed “Tier 1A.” Algal and plant Tier 1B orthologs include CCP1 orthologs from the alga Ettlia oleoabundans and plant Zea nicaraguensis, suggesting the intriguing possibility that the plant Tier 1B CCP1 orthologs may have resulted from horizontal gene transfer from Ettlia oleoabundans or related algae. The observation that the LCID/E orthologs encoded by the various species of land plants appear to be highly similar to the LCID/E ortholog of Ettlia oleoabundans suggests the possibility that the LCID/E proteins encoded by the various species of land plants also may have resulted from horizontal gene transfer from Ettlia oleoabundans or related algae. This also suggests that Zea nicaraguensis and the other plant species encoding Tier 1B CCP1 orthologs and/or LCID/E orthologs may serve as sources of CCP1 orthologs and LCID/E orthologs that are proximally derived from land plants, rather than from algae, thus decreasing regulatory concerns and risk associated with genetic modification of crops, while providing increases in crop yield comparable to those observed for CCP1 of Chlamydomonas reinhardtii and CCP1 orthologs derived from other algae, and that potentially may be observed for LCID and LCIE of Chlamydomonas reinhardtii and/or LCID/E orthologs of other eukaryotic algae.

Results from a BLAST search that reveals that various land plants encode LCID/E orthologs are shown in TABLE 7.

TABLE 7
Protein orthologs of C. reinhardtii LCIE identified in land plants.

| Accession | Sequence | Score | E value |
|---|---|---|---|
| GBZQ01030305.1 | TSA: Zea nicaraguensis | 365 | 6.00E−118 |
| | comp52926_c0_seq1 trans. . . | | |
| GEZQ01052238.1 | TSA: Cosmos bipinnatus | 305 | 3.00E−94 |
| | c78291.graph_c6 transcr. . . | | |
| GBJI01008921.1 | TSA: Arachis hypogaea var. | 270 | 3.00E−82 |
| | vulgaris Contig9000. . . | | |
| GEZQ01025791.1 | TSA: Cosmos bipinnatus | 248 | 3.00E−74 |
| | c34659.graph_c0 transcr. . . | | |
| GEZQ01020296.1 | TSA: Cosmos bipinnatus | 228 | 1.00E−66 |
| | c24362.graph_c0 transcr. . . | | |
| GEZT01009142.1 | TSA: Solanum prinophyllum | 222 | 9.00E−68 |
| | c8880_g1_i1 transcri. . . | | |
| GBZQ01047102.1 | TSA: Zea nicaraguensis | 194 | 7.00E−55 |
| | comp66612_c0_seq5 trans. . . | | |
| GCIB01149448.1 | TSA: Colobanthus quitensis | 179 | 1.00E−51 |
| | Colobanthus_quitens. . . | | |
| GEZQ01035651.1 | TSA: Cosmos bipinnatus | 164 | 2.00E−45 |
| | c59376.graph_c0 transcr. . . | | |
| GBZQ01003577.1 | TSA: Zea nicaraguensis | 161 | 6.00E−45 |
| | comp4315_c1_seq1 transc. . . | | |
| GEZQ01028890.1 | TSA: Cosmos bipinnatus | 159 | 8.00E−41 |
| | c40190.graph_c0 transcr. . . | | |
| GEZQ01052236.1 | TSA: Cosmos bipinnatus | 149 | 2.00E−37 |
| | c78291.graph_c3 transcr. . . | | |
| GEBH01256214.1 | TSA: Poa pratensis IXRNA007_ | 145 | 2.00E−39 |
| | contig_270617 tran. . . | | |
| GFBT01099195.1 | TSA: Nymphoides peltata | 140 | 1.00E−33 |
| | treatTR55412cOg1i1 tra. . . | | |
| GFBT01098813.1 | TSA: Nymphoides peltata | 134 | 1.00E−31 |
| | treatTR66405c1g1i1 tra. . . | | |
| GEZQ01049458.1 | TSA: Cosmos bipinnatus | 125 | 1.00E−28 |
| | c75738.graph_c0 transcr. . . | | |
| GFMV01082673.1 | TSA: Camellia sinensis | 124 | 8.00E−32 |
| | c181886.graph_c0 transc. . . | | |
| GCHX01036611.1 | TSA: Picea glauca PG29_ | 121 | 1.00E−29 |
| | BFMXY_Trinity_Pasafly_c. . . | | |
| GEZQ01040447.1 | TSA: Cosmos bipinnatus | 120 | 8.00E−29 |
| | c66104.graph_c0 transcr. . . | | |
| GCAA01033789.1 | TSA: Zea nicaraguensis | 118 | 3.00E−28 |
| | a451365_9 transcribed R. . . | | |
| GBIX01040188.1 | TSA: Arachis hypogaea var. | 115 | 4.00E−27 |
| | vulgaris Contig_403. . . | | |
| GCIB01142293.1 | TSA: Colobanthus quitensis | 108 | 3.00E−24 |
| | Colobanthus_quitens. . . | | |
| GCIB01120345.1 | TSA: Colobanthus quitensis | 107 | 2.00E−23 |
| | Colobanthus_quitens. . . | | |
| GEDP01309010.1 | TSA: Triticum polonicum | 103 | 1.00E−22 |
| | comp6750_c1 transcribe... | | |
| GEBH01365933.1 | TSA: Poa pratensis IXRNA007_ | 102 | 3.00E−23 |
| | contig_386926 tran. . . | | |
| GCKF01054446.1 | TSA: Araucaria cunninghamii | 102 | 3.00E−23 |
| | Ref_Hoop_Pine_Tran. . . | | |
| GEBH01110972.1 | TSA: Poa pratensis IXRNA007_ | 102 | 1.00E−23 |
| | contig_114479 tran. . . | | |
| GFMV01104148.1 | TSA: Camellia sinensis | 100 | 1.00E−22 |
| | c204739.graph_c0 transc. . . | | |
| GEZQ01052237.1 | TSA: Cosmos bipinnatus | 96.3 | 1.00E−21 |
| | c78291.graph_c5 transcr. . . | | |
| GFBT01128161.1 | TSA: Nymphoides peltata | 92.8 | 2.00E−19 |
| | treatTR27027c0g1i1 tra. . . | | |
| GCKF01013257.1 | TSA: Araucaria cunninghamii | 87 | 5.00E−18 |
| | Ref_Hoop_Pine_Tran... | | |
| GEBH01350073.1 | TSA: Poa pratensis IXRNA007_ | 85.9 | 1.00E−17 |
| | contig_370810 tran. . . | | |
| GACA01042850.1 | TSA: Pohlia nutans Moss_ | 85.1 | 4.00E−16 |
| | FIO_Consensus91451 mRN. . . | | |
| GEBH01216780.1 | TSA: Poa pratensis IXRNA007_ | 84.7 | 6.00E−16 |
| | contig_227368 tran. . . | | |
| GEZT01014111.1 | TSA: Solanum prinophyllum | 84.3 | 2.00E−15 |
| | c13770_g1_i1 transcr. . . | | |
| GEBH01238057.1 | TSA: Poa pratensis IXRNA007_ | 82 | 3.00E−16 |
| | contig_250559 tran. . . | | |
| GFBT01129453.1 | TSA: Nymphoides peltata | 81.6 | 2.00E−15 |
| | treatTR16147cOg1i1 tra. . . | | |
| GFBT01133705.1 | TSA: Nymphoides peltata | 79.3 | 8.00E−15 |
| | treatTR66405c2g1i1 tra. . . | | |
| GEZQ01025871.1 | TSA: Cosmos bipinnatus | 79 | 3.00E−14 |
| | c34815.graph_c0 transcr. . . | | |
| GBEN01078670.1 | TSA: Elodea nuttallii | 77.4 | 3.00E−13 |
| | Locus_25806_Transcript_1. . . | | |
| GFBT01118477.1 | TSA: Nymphoides peltata | 75.1 | 1.00E−12 |
| | treatTR66405cOg1i1 tra. . . | | |
| GEBH01212216.1 | TSA: Poa pratensis IXRNA007_ | 71.2 | 7.00E−12 |
| | contig_222432 tran. . . | | |
| GEBH01288613.1 | TSA: Poa pratensis IXRNA007_ | 65.9 | 3.00E−10 |
| | contig_306530 tran. . . | | |

Exemplary LCID/E orthologs from land plants include an LCID/E protein of Zea nicaraguensis of SEQ ID NO: 6, an LCID/E protein of Cosmos bipinnatus of SEQ ID NO: 7, and an LCID/E protein of Nymphoides peltata of SEQ ID NO: 8.

Results of a BLAST search that reveals that the LCID/E protein of Zea nicaraguensis is closely related to the LCID/E ortholog of Ettlia oleoabundans is shown in TABLE 8.

TABLE 8
Results of BLAST search to identify eukaryotic algal
sequences related to LCID/E protein of Zea nicaraguensis.

| | Total | Query | E | | |
|---|---|---|---|---|---|
| Description | score | cover | value | Ident. | Accession |
| TSA: Ettlia | 506 | 67% | 7e−177 | 52.1% | GEEU01065332.1 |
| oleoabundans | | | | | |
| 11004_c0_seq1 | | | | | |
| transcribed RNA | | | | | |
| sequence | | | | | |
| Chlamydomonas | 498 | 98% | 1e−171 | 50.6% | XM_001692140.1 |
| reinhardtii strain | | | | | |
| CC-503 cw92 | | | | | |
| mt + LCID | | | | | |
| Chlamydomonas | 498 | 98% | 1e−171 | 50.6% | DQ657195.1 |
| reinhardtii | | | | | |
| LciD mRNA, | | | | | |
| complete cds | | | | | |
| Volvox carteri f. | 482 | 98% | le−168 | 51.4% | XM_002954362.1 |
| nagariensis low-CO2 | | | | | |
| inducible protein | | | | | |
| (1ciB), mRNA | | | | | |
| Chlamydomonas | 498 | 98% | 2e−168 | 50.6% | DQ657194.1 |
| reinhardtii | | | | | |
| LciD mRNA, | | | | | |
| complete cds | | | | | |
| TSA: Chlorella | 416 | 78% | 2e−139 | 42.2% | GAPD01044302.1 |
| sorokiniana | | | | | |
| comp12448_c2_ | | | | | |
| seq14 transcribed | | | | | |
| RNA sequence | | | | | |
| TSA: Oophila | 417 | 91% | 3e−139 | 43.2% | GFMX01032203.1 |
| amblystomatis | | | | | |
| c19515_g1_i3 | | | | | |
| transcribed RNA | | | | | |
| sequence | | | | | |
| TSA: Chlorella | 417 | 78% | 5e−139 | 44.6% | GAPD01044303.1 |
| sorokiniana | | | | | |
| comp12448_c2_ | | | | | |
| seq15 transcribed | | | | | |
| RNA sequence | | | | | |
| TSA: Oophila | 414 | 91% | 3e−138 | 43.2% | GFMX01032201.1 |
| amblystomatis | | | | | |
| c19515_g1_i1 | | | | | |
| transcribed RNA | | | | | |
| sequence | | | | | |
| Chlamydomonas | 411 | 89% | 3e−137 | 48.1% | XM_001691171.1 |
| reinhardtii strain | | | | | |
| CC-503 cw92 mt+ | | | | | |
| Chlamydomonas | 411 | 89% | 4e−137 | 48.1% | AB168094.1 |
| reinhardtii LciC | | | | | |
| mRNA for | | | | | |
| low-CO2 inducible | | | | | |
| protein LCIC, | | | | | |
| complete cds | | | | | |
| Volvox carteri f. | 414 | 89% | 1e−136 | 47.1% | XM_002951460.1 |
| nagariensis GON30 | | | | | |
| protein (gon30), | | | | | |
| mRNA | | | | | |
| Chlamydomonas | 397 | 76% | 6e−136 | 44.2% | XM_001692241.1 |
| reinhardtii strain | | | | | |
| CC-503 cw92 | | | | | |
| mt + LCIE | | | | | |
| TSA: Oophila | 410 | 81% | 2e−135 | 45.8% | GFMX01032202.1 |
| amblystomatis | | | | | |
| c19515_g1_i2 | | | | | |
| transcribed RNA | | | | | |
| sequence | | | | | |
| TSA: Oophila | 410 | 81% | 5e−135 | 49.6% | GFMZ01016772.1 |
| amblystomatis | | | | | |
| c194112_g1_i1 | | | | | |
| transcribed RNA | | | | | |
| sequence | | | | | |
| TSA: Oophila | 407 | 80% | 2e−134 | 45.8% | GFMX01032204.1 |
| amblystomatis | | | | | |
| c19515_g1_i4 | | | | | |
| transcribed RNA | | | | | |
| sequence | | | | | |
| TSA: Chlamydomonas | 397 | 86% | 1e−133 | 46.3% | GBAH01004893.1 |
| acidophila | | | | | |
| contig07687 | | | | | |
| transcribed | | | | | |
| RNA sequence | | | | | |
| TSA: Chlorella | 414 | 78% | 6e−133 | 44.0% | GAPD01044291.1 |
| sorokiniana | | | | | |
| comp12448_c2_seq3 | | | | | |
| transcribed RNA | | | | | |
| sequence | | | | | |
| Volvox carteri f. | 395 | 90% | 6e−130 | 44.5% | XM_002957296.1 |
| nagariensis | | | | | |
| hypothetical protein, | | | | | |
| mRNA | | | | | |
| Chlamydomonas | 391 | 81% | 1e−129 | 44.6% | XM_001698292.1 |
| reinhardtii low-CO2- | | | | | |
| inducible protein | | | | | |
| (LCIB) mRNA, | | | | | |
| complete cds | | | | | |

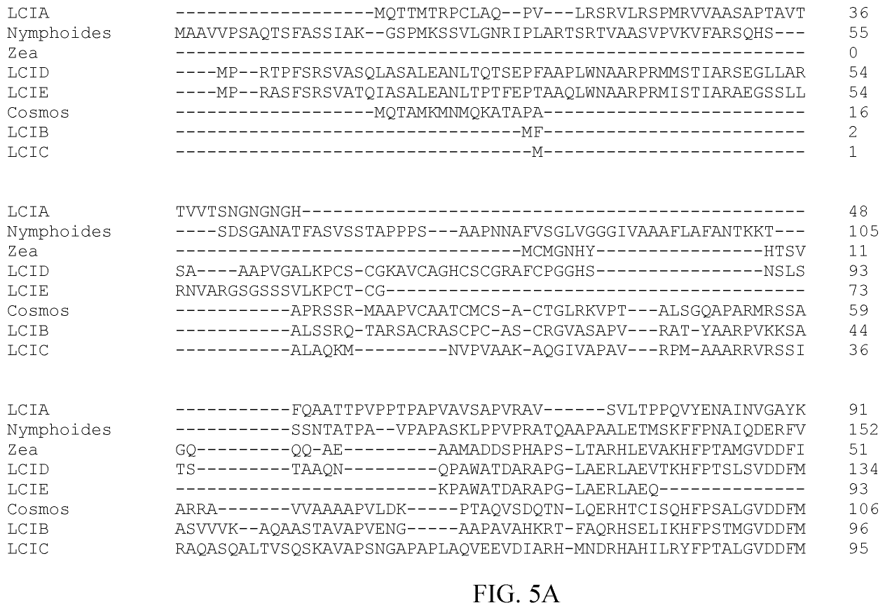

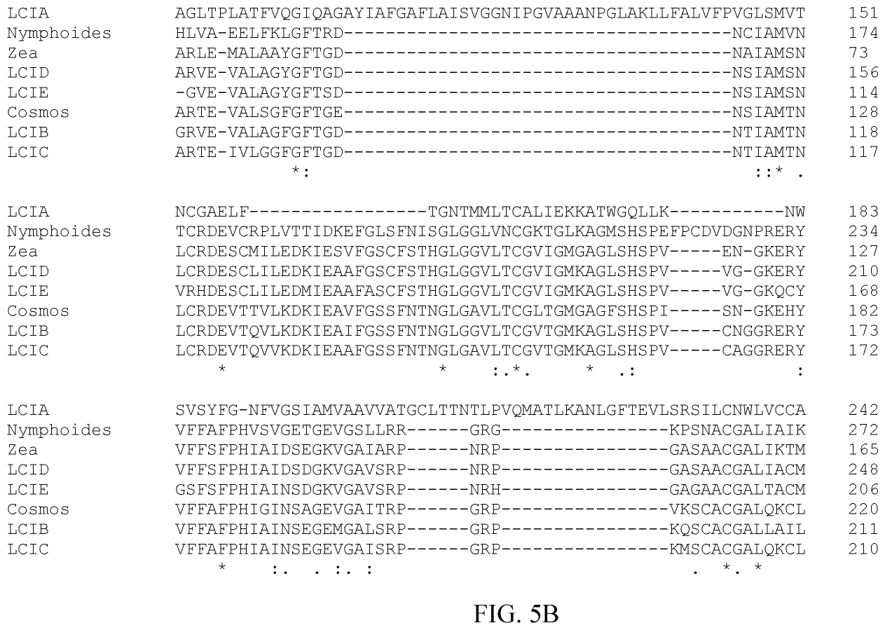

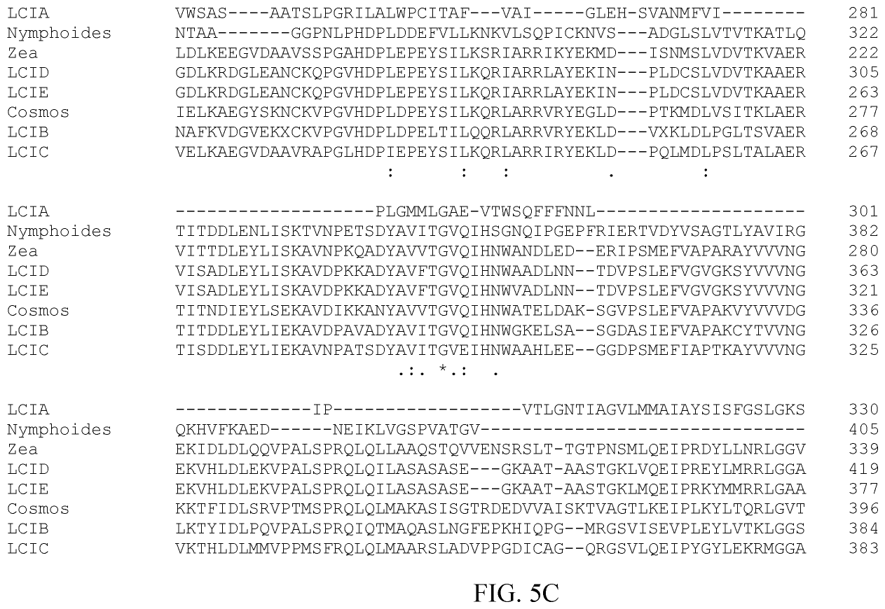

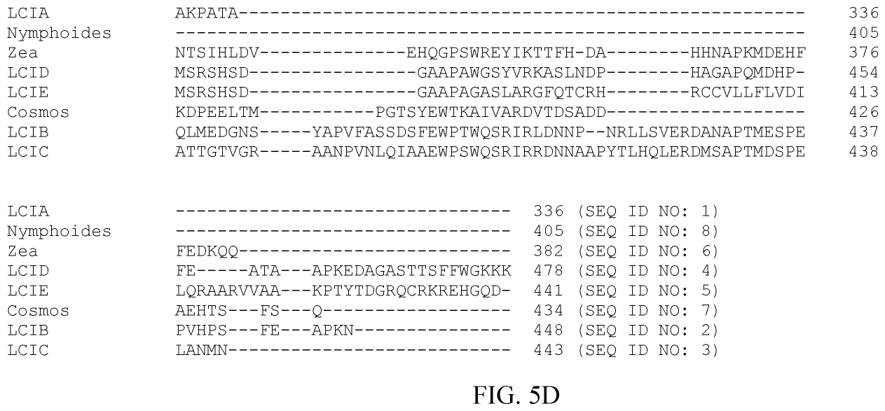

A multiple sequence alignment of the C. reinhardtii LCIA (SEQ ID NO: 1), LCIB (SEQ ID NO: 2), LCIC (SEQ ID NO: 3), LCD (SEQ ID NO: 4), and LCIE (SEQ ID NO: 5) proteins and LCID/E orthologs of Zea nicaraguensis (SEQ ID NO: 6), Cosmos bipinnatus (SEQ ID NO: 7), and Nymphoides peltata (SEQ ID NO: 8) is shown in FIG. 5.

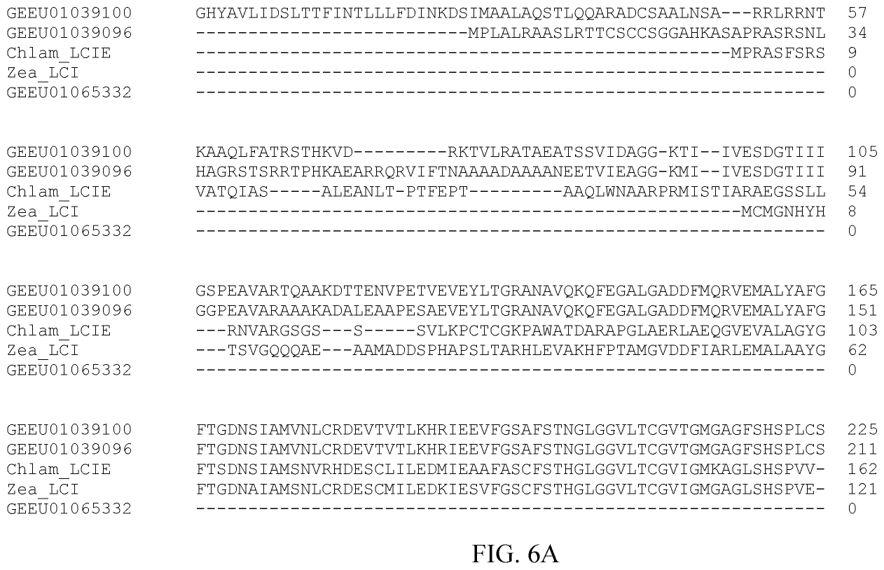

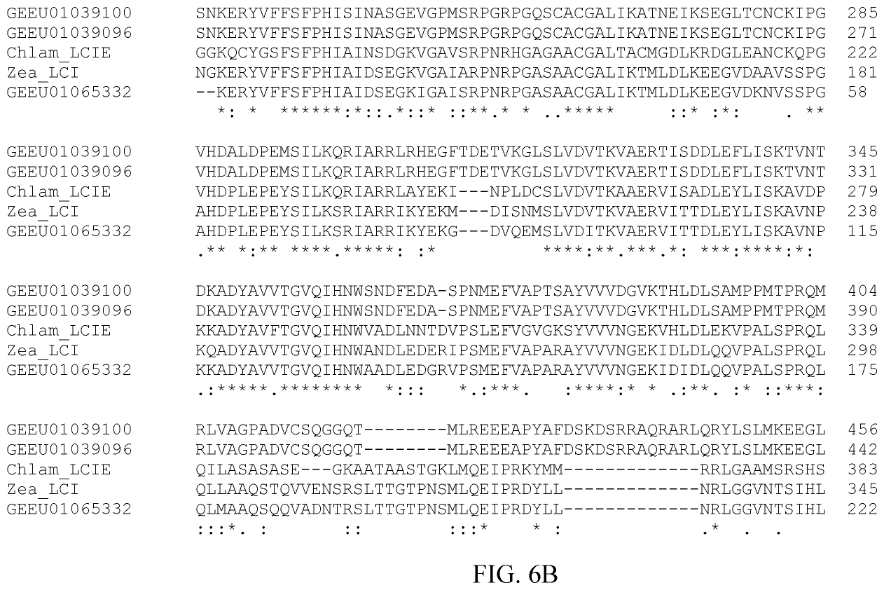

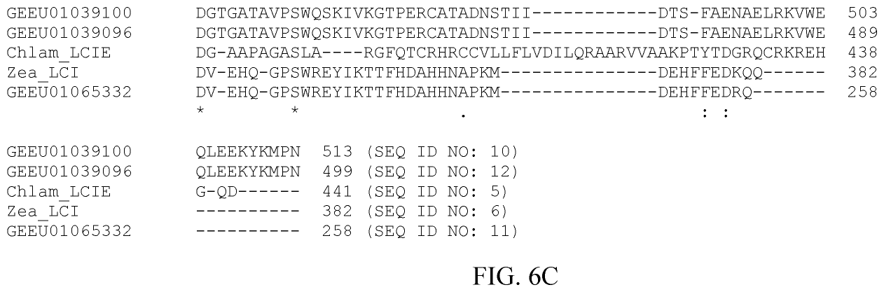

A multiple sequence alignment of the C. reinhardtii LCIE protein (SEQ ID NO: 5), LCID/E orthologs of Ettlia oleoabundans (SEQ ID NO: 10, SEQ ID NO: 11, and SEQ ID NO: 12), and the LCID/E ortholog of Zea nicaraguensis (SEQ ID NO: 6) is shown in FIG. 6.

### Example 9. Use of Pyruvate Carboxylase in C3 Leaf to Enhance CCP1 Function

Pyruvate carboxylase (also termed PYC, corresponding to EC 6.4.1.1) catalyzes the carboxylation of pyruvate to oxaloacetate (also termed OAA):

ATP+Pyruvate+HCO₃⁻→ADP+Pᵢ+OAA=ΔG′^{m}=5.9 kJ/mol

There are no conclusive reports of higher plants containing pyruvate carboxylase genes, though they are commonly found in algae, bacteria, fungi, and higher animals. Pyruvate carboxylase is commonly used in gluconeogenesis to effect the conversion of pyruvate to phosphoenolpyruvate (also termed PEP), in tandem with phosphoenolpyruvate carboxykinase (also termed PEPCK, corresponding to EC 4.1.1.49) or a similar enzyme:

ATP+OAA=ADP+PEP+CO₂ΔG′^{m}=−11.7 kJ/mol

If CCP1 expels malate from the mitochondrion into the cytosol in conjunction with oxaloacetate uptake into the mitochondrion under photorespiratory conditions in a C3 leaf, then ideally all of this malate would be used by the peroxisome to generate NADH for use by hydroxypyruvate reductase, a key enzyme in photorespiration. In reality, however, a significant amount of this malate might be converted to pyruvate by a form of malic enzyme in the cytosol, liberating CO₂ and NAD(P)H:

Malate+NAD(P)+=Pyruvate+CO₂+NAD(P)H ΔG′^{m}=−4.1 kJ/mol

This could occur if hydroxypyruvate reductase in the peroxisome cannot keep up with the influx of malate as its NADH source (largely because malate dehydrogenase is thermodynamically difficult to utilize in the electron-extracting direction). It also may occur because the plant is not accustomed to the increased flux to cytosolic malate that CCP1 provides and either has residual malic enzyme activity for defense response or biosynthetic purposes or actually upregulates cytosolic malic enzyme in response to the increased malate to prevent its accumulation.

If cytosolic malate is indeed converted to excess pyruvate by malic enzyme, the cell has little recourse but to use the TCA cycle to degrade the pyruvate to CO₂, given the difficulty of recovering pyruvate as PEP. Pyruvate carboxylase, however, can recycle the pyruvate to oxaloacetate as a partner for malate in the malate-oxaloacetate shuttle system (or ultimately to aspartate or asparagine for transport to the seed) and conserve CO₂ in the process.

Pyruvate carboxylase increases the theoretical yield in a photorespiring C3 leaf with or without a CCP1-like activity, according to flux-balance (stoichiometric) analysis. When pyruvate carboxylase is present, theoretical yields are not affected by malic enzyme flux. When pyruvate carboxylase is absent, however, a malic enzyme flux corresponding to half the CCP1 flux lowers the theoretical biomass yield in leaf or seed by more than 10% while necessitating about twice the initial flux through CCP1. The actual yield differential could be higher than 10% because the flux through malic enzyme could be greater than estimated here, or the higher CCP1 flux necessitated by malic enzyme may not be attainable.

There are also kinetic reasons why pyruvate carboxylase can contribute to biomass yield. During photosynthesis, 3-phosphoglycerate (also termed 3PG) can accumulate because of the unfavorability of the phosphoglycerate kinase reaction, which converts 3-phosphoglycerate to 1,3-bisphosphoglycerate (also termed 13BPG). Some of this 3-phosphoglycerate can be converted ultimately to pyruvate instead of sugar, and once again the plant cell is compelled to waste this carbon via the TCA cycle because it cannot recover the pyruvate.

Pyruvate carboxylase can thus be a valuable addition to a photosynthesizing leaf, especially when paired with CCP1 or a like activity.

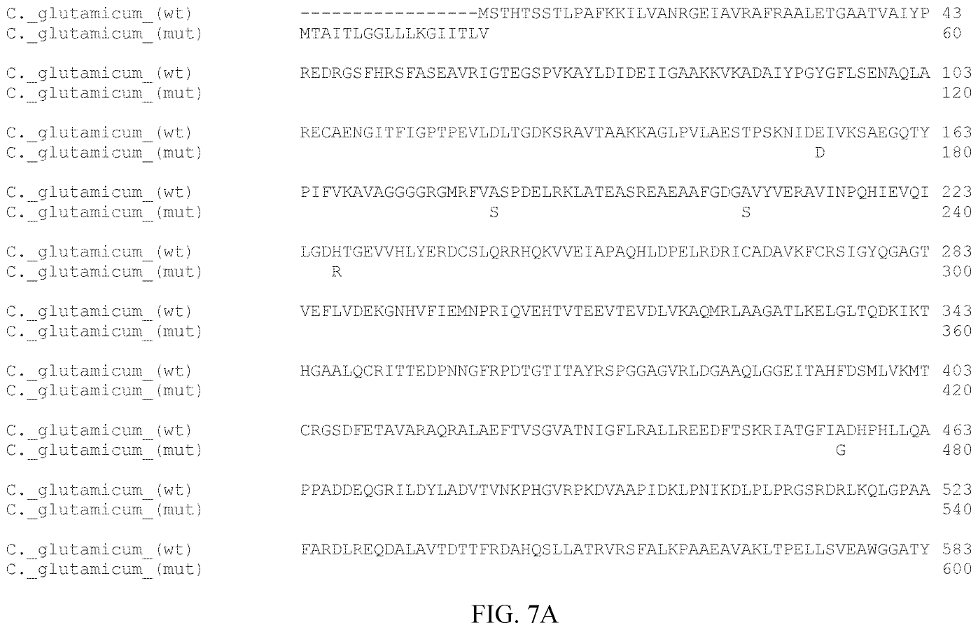

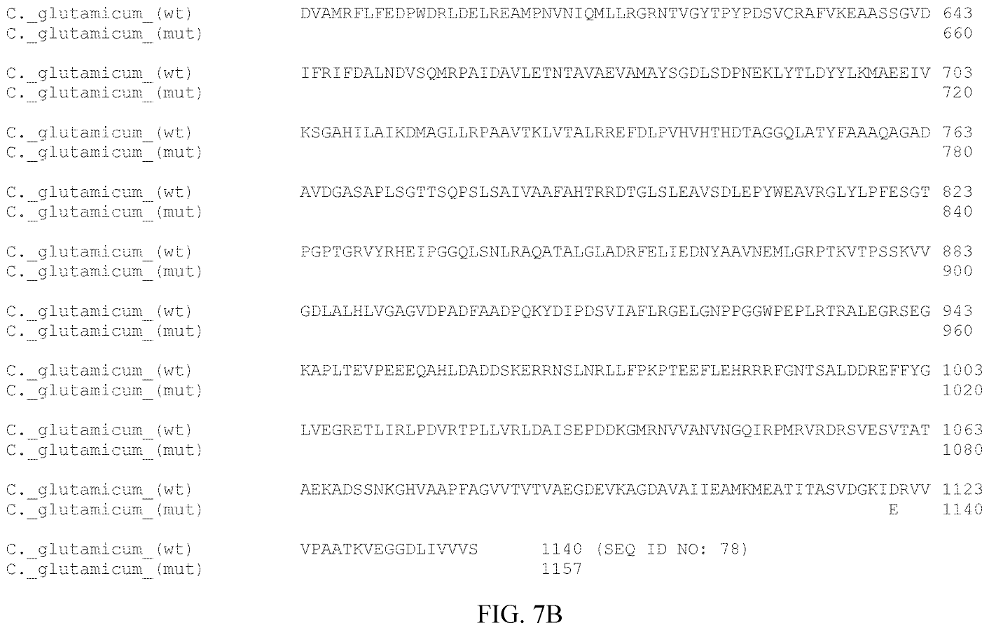

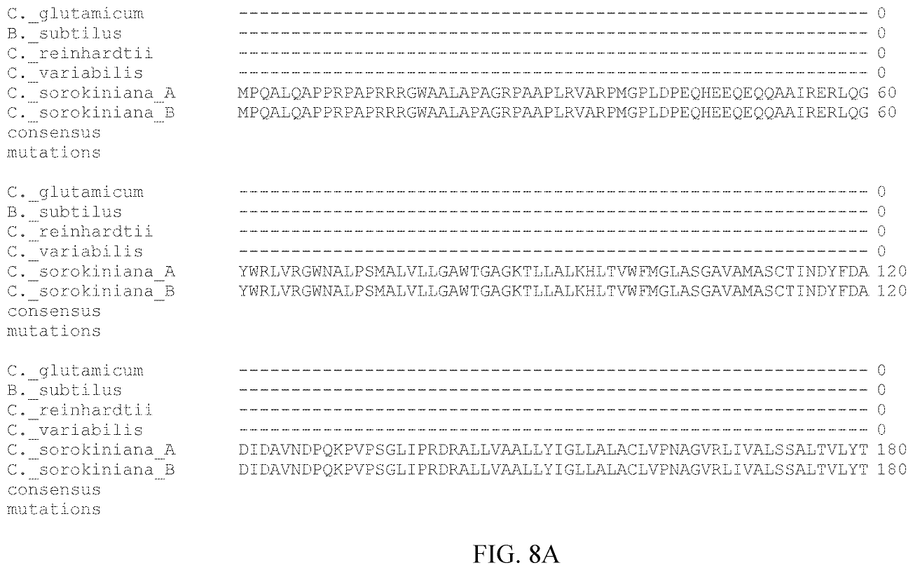

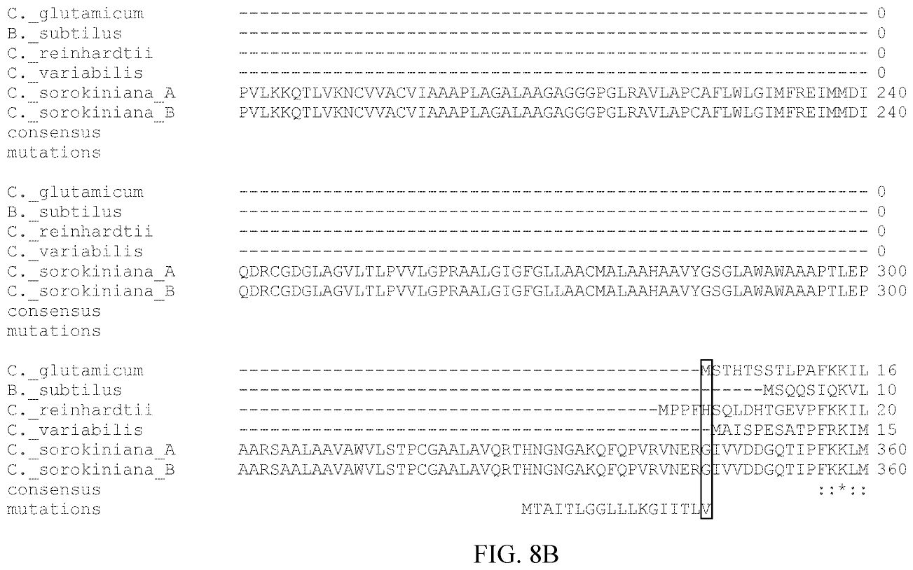

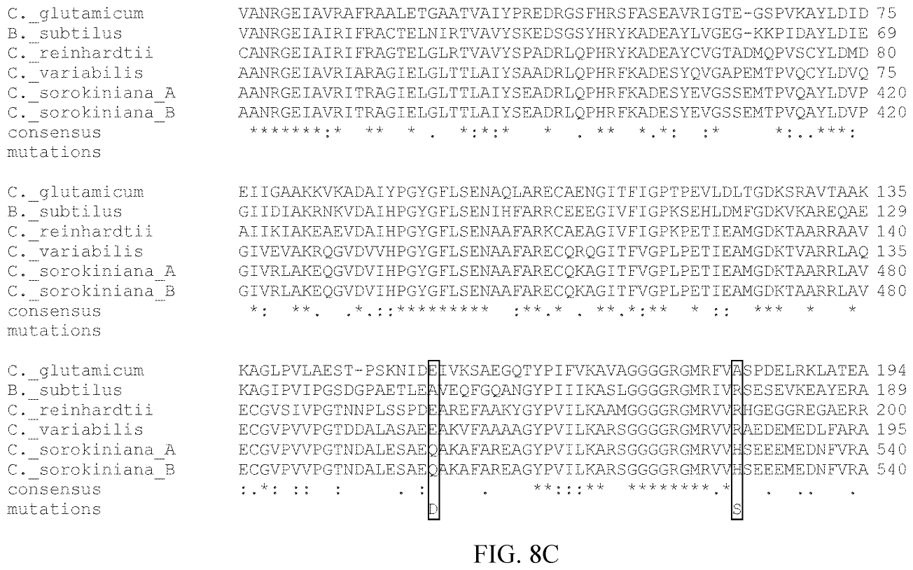

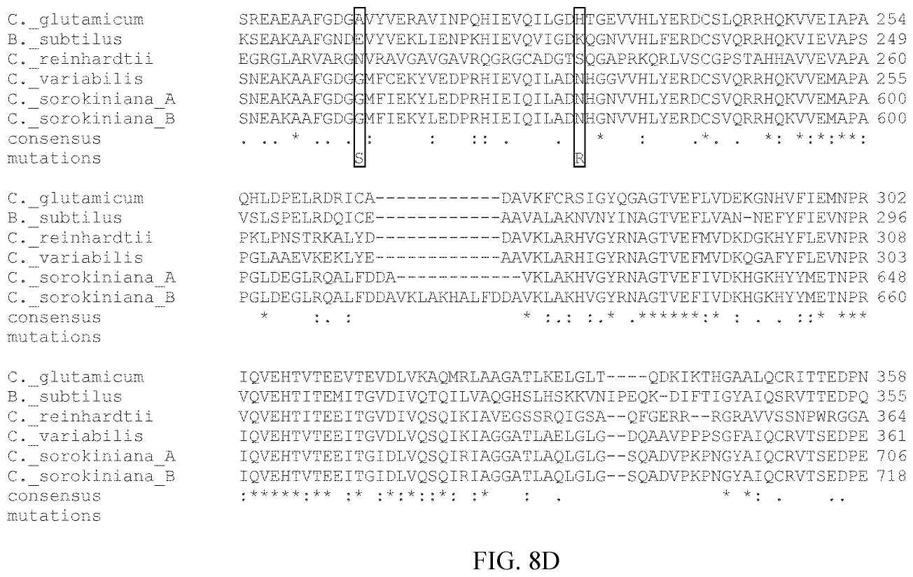

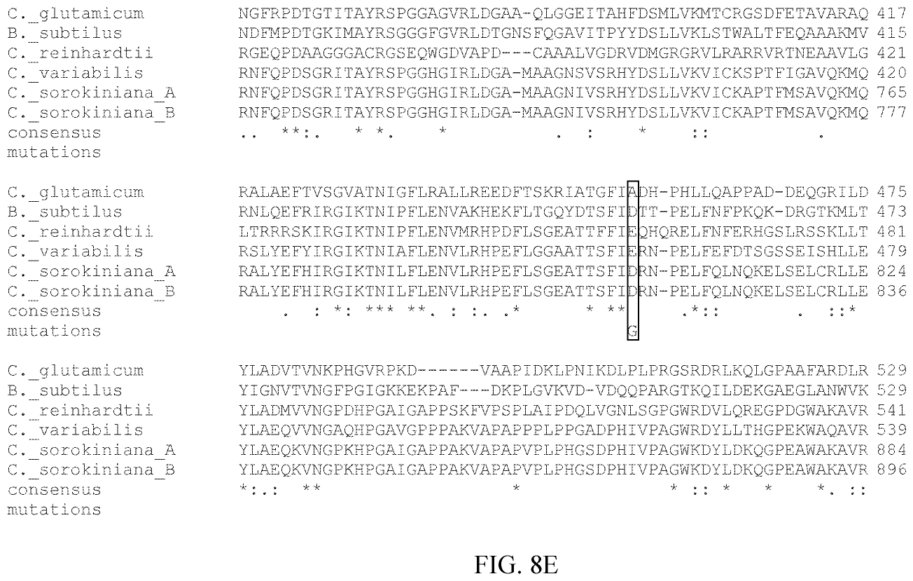

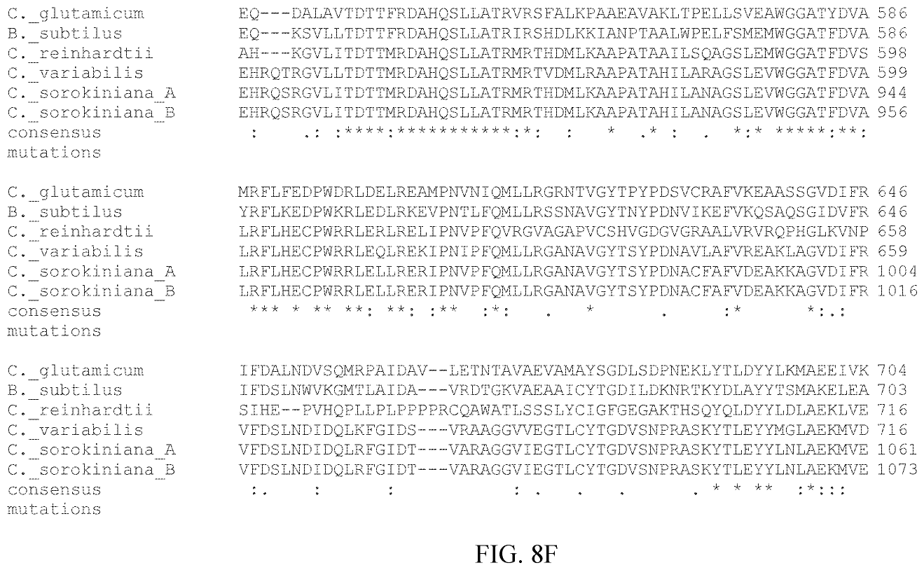

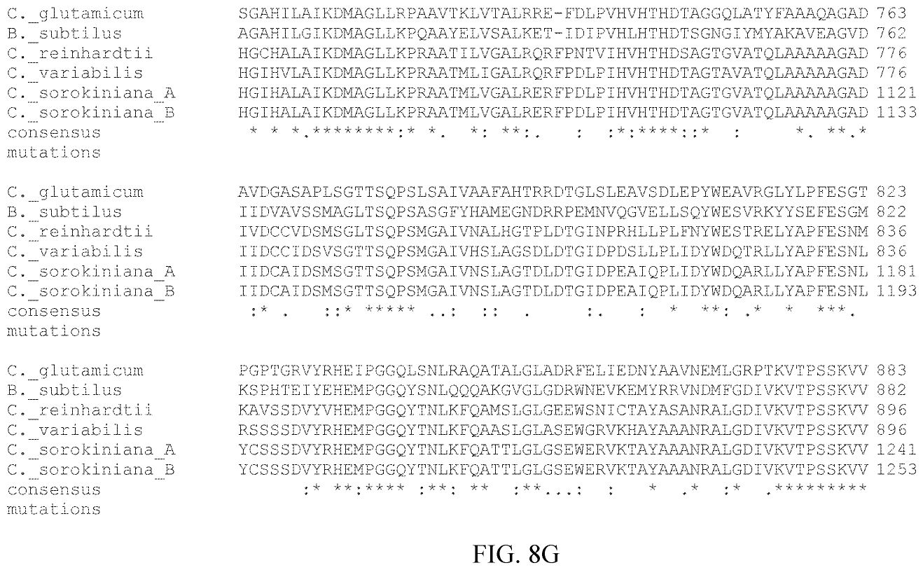

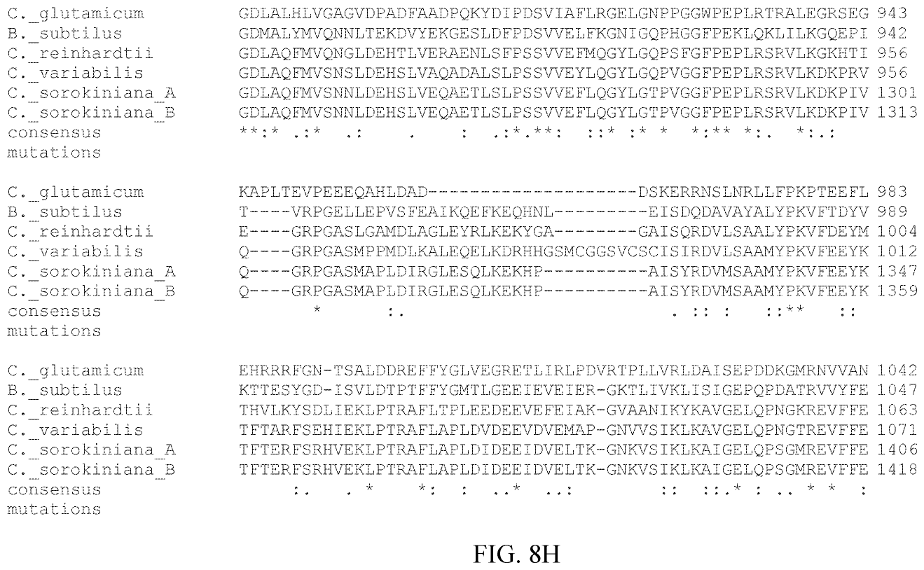

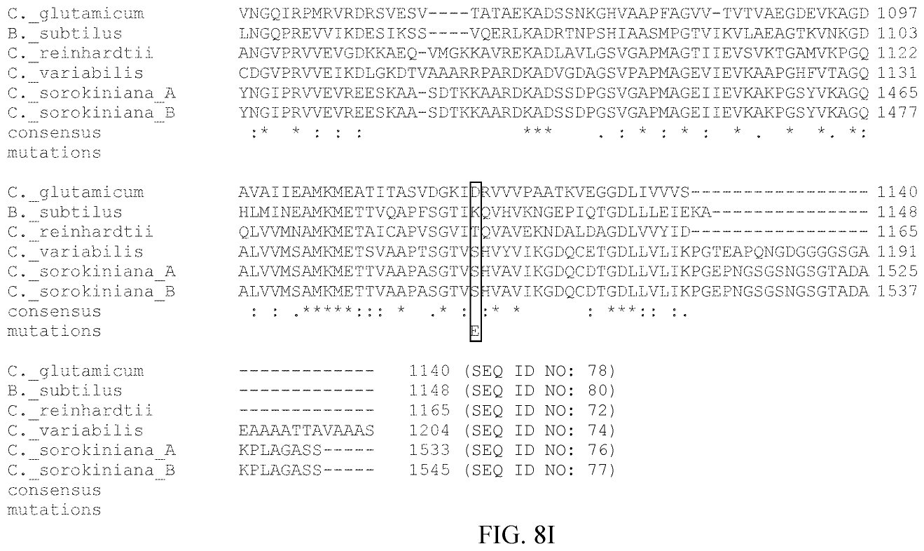

Exemplary pyruvate carboxylase genes and enzymes useful for contributing to biomass yield are provided in FIG. 7A-B, FIG. 8A-I, and TABLE 9.

TABLE 9
Pyruvate carboxylase (EC 6.4.1.1) genes and proteins from algae.

| | | GenBank |
|---|---|---|
| Organism | Locus | Accession |
| Chlamydomonas reinhardtii | CHLREDRAFT_112730 | XP_001696348.1 |
| | (PYC1) | |
| Chlorella variabilis | CHLNCDRAFT_138936 | XP_005844530.1 |
| Chlorella sorokiniana | C2E21_8932 | PRW20525.1 |
| | (isoform A) | |

FIG. 7A-B shows a pairwise alignment of wild-type pyruvate carboxylase of Corynebacterium glutamicum (SEQ ID NO. 78) and a mutated pyruvate carboxylase of Corynebacterium glutamicum that is desensitized to feedback inhibition from aspartic acid (SEQ ID NO. 79) according to CLUSTAL O(1.2.4). The wild-type pyruvate carboxylase of Corynebacterium glutamicum can be a valuable addition as discussed above. The mutated pyruvate carboxylase of Corynebacterium glutamicum that is desensitized to feedback inhibition from aspartic acid may provide a particular advantage when cells of a plant are making high amounts of aspartate to send to the phloem. The complete sequence of the wild-type pyruvate carboxylase and differences between the mutated pyruvate carboxylase and the wild-type pyruvate carboxylase are shown.

FIG. 8A-I shows a multiple sequence alignment of pyruvate carboxylase of Corynebacterium glutamicum (SEQ ID NO. 78), Bacillus subtilus (SEQ ID NO: 80), Chlamydomonas reinhardtii (SEQ ID NO: 72), Chlorella variabilis (SEQ ID NO: 74), Chlorella sorokiniana (isoform A) (SEQ ID NO: 76), and Chlorella sorokiniana (isoform B) (SEQ ID NO: 77) according to CLUSTAL 0(1.2.4). Positions of mutations of the mutated pyruvate carboxylase of Corynebacterium glutamicum that is desensitized to feedback inhibition from aspartic acid (SEQ ID NO: 79) relative to the other pyruvate carboxylase sequences also are shown.

TABLE 9 provides locus and GenBank Accession information for pyruvate carboxylate genes and proteins from Chlamydomonas reinhardtii, Chlorella variabilis, and Chlorella sorokiniana.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

### Reference to a “Seuqnce Listing,” a Table, or a Computer Program Listing Appendix Submitted as an ASCII Text File

The material in the ASCII text file, named “YTEN-57558WO-sequence-listing_ST25.txt”, created Oct. 26, 2018, file size of 319,488 bytes, is hereby incorporated by reference.

1. A genetically engineered land plant that expresses an LCID/E protein, the genetically engineered land plant comprising a modified gene for the LCID/E protein, wherein:
the LCID/E protein comprises (i) LCD of Chlamydomonas reinhardtii of SEQ ID NO: 4, (ii) LCIE of Chlamydomonas reinhardtii of SEQ ID NO: 5, or (iii) an algal or plant ortholog of LCID/E;
the LCID/E protein is localized to chloroplasts of the genetically engineered land plant based on a plastidial targeting signal;
the modified gene for the LCID/E protein comprises (i) a promoter and (ii) a nucleic acid sequence encoding the LCID/E protein;
the promoter is non-cognate with respect to the nucleic acid sequence encoding the LCID/E protein; and
the modified gene for the LCID/E protein is configured such that transcription of the nucleic acid sequence encoding the LCID/E protein is initiated from the promoter and results in expression of the LCID/E protein.

2. (canceled)

3. (canceled)

4. The genetically engineered land plant of claim 1, wherein the LCID/E protein comprises the algal or plant ortholog of LCID/E based on comprising: (i) one or more LCID/E signature sequences of (a) FSFPHI (SEQ ID NO: 13) at position 213-218, (b) ACGAL (SEQ ID NO: 14) at position 240-244, (c) ADYAV (SEQ ID NO: 15) at position 324-328, or (d) TGVQIHNW (SEQ ID NO: 16) at position 330-337, with numbering of positions relative to LCD of Chlamydomonas reinhardtii of SEQ ID NO: 4, and (ii) an overall identity of at least 60%.

5. The genetically engineered land plant of claim 1, wherein the LCID/E protein comprises at least one of (a) an LCID/E protein of Zea nicaraguensis, (b) an LCID/E protein of Cosmos bipinnatus, or (c) an LCID/E protein of Nymphoides peltata.

6. (canceled)

7. The genetically engineered land plant of claim 1, wherein the LCID/E protein comprises at least one of (a) an LCID/E protein of Zea nicaraguensis of SEQ ID NO: 6, (b) an LCID/E protein of Cosmos bipinnatus of SEQ ID NO: 7, or (c) an LCID/E protein of Nymphoides peltata of SEQ ID NO: 8.

8. The genetically engineered land plant of claim 7, wherein the LCID/E protein comprises an LCID/E protein of Zea nicaraguensis of SEQ ID NO: 6.

9-11. (canceled)

12. The genetically engineered land plant of claim 1, wherein the promoter is a constitutive promoter.

13. The genetically engineered land plant of claim 1, wherein the promoter is a seed-specific promoter.

14. (canceled)

15. (canceled)

16. The genetically engineered land plant of claim 1, wherein the genetically engineered land plant has a CO₂ assimilation rate that is at least 5% higher, at least 10% higher, at least 20% higher, or at least 40% higher, than for a corresponding reference land plant that does not comprise the modified gene for the LCID/E protein.

17. The genetically engineered land plant of claim 1, wherein the genetically engineered land plant has a transpiration rate that is at least 5% lower, at least 10% lower, at least 20% lower, or at least 40% lower, than for a corresponding reference land plant that does not comprise the modified gene for the LCID/E protein.

18. The genetically engineered land plant of claims 1, wherein the genetically engineered land plant has a seed yield that is at least 5% higher, at least 10% higher, at least 20% higher, at least 40% higher, at least 60% higher, or at least 80% higher, than for a corresponding reference land plant that does not comprise the modified gene for the LCID/E protein.

19. (canceled)

20. (canceled)

21. The genetically engineered land plant of claim 1, wherein the genetically engineered land plant is a food crop plant selected from the group consisting of maize, wheat, oat, barley, soybean, millet, sorghum, potato, pulse, bean, tomato, and rice.

22. (canceled)

23. The genetically engineered land plant of claim 1, wherein the genetically engineered land plant is a forage crop plant selected from the group consisting of silage corn, hay, and alfalfa.

24. (canceled)

25. The genetically engineered land plant of claim 1, wherein the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

26. The genetically engineered land plant of claim 1, wherein the genetically engineered land plant further expresses a CCP1 mitochondrial transporter protein, the genetically engineered land plant comprising a modified gene for the CCP1 mitochondrial transporter protein, further wherein:
the CCP1 mitochondrial transporter protein comprises: (i) CCP1 of Chlamydomonas reinhardtii of SEQ ID NO: 9 or (ii) an ortholog of CCP1;
the CCP1 mitochondrial transporter protein is localized to mitochondria of the genetically engineered land plant based on a mitochondrial targeting signal;
the modified gene for the CCP1 mitochondrial transporter protein comprises (i) another promoter and (ii) a nucleic acid sequence encoding the CCP1 mitochondrial transporter protein;
the other promoter is non-cognate with respect to the nucleic acid sequence encoding the CCP1 mitochondrial transporter protein; and
the modified gene for the CCP1 mitochondrial transporter protein is configured such that transcription of the nucleic acid sequence encoding the CCP1 mitochondrial transporter protein is initiated from the other promoter and results in expression of the CCP1 mitochondrial transporter protein.

27. (canceled)

28. (canceled)

29. The genetically engineered land plant of claim 26, wherein the ortholog of CCP1 comprises a plant CCP1 ortholog.

30. The genetically engineered land plant of claim 29, wherein the plant CCP1 ortholog comprises a CCP1 ortholog of Erigeron breviscapus of SEQ ID NO: 57, Zea nicaraguensis of SEQ ID NO: 58, Poa pratensis of SEQ ID NO: 59, Cosmos bipinnatus of SEQ ID NO: 60, Glycine max of SEQ ID NO: 61, Zea mays of SEQ ID NO: 62, Oryza sativa of SEQ ID NO: 63, Triticum aestivum of SEQ ID NO: 64, Sorghum bicolor of SEQ ID NO: 65, or Solanum tuberosum of SEQ ID NO: 66.

31. The genetically engineered land plant of claim 1, wherein the genetically engineered land plant further expresses a pyruvate carboxylase, the genetically engineered land plant comprising a modified gene for the pyruvate carboxylase, further wherein:
the modified gene for the pyruvate carboxylase comprises (i) a further promoter and (ii) a nucleic acid sequence encoding the pyruvate carboxylase;
the further promoter is non-cognate with respect to the nucleic acid sequence encoding the pyruvate carboxylase; and
the modified gene for the pyruvate carboxylase is configured such that transcription of the nucleic acid sequence encoding the pyruvate carboxylase is initiated from the further promoter and results in expression of the pyruvate carboxylase.

32. The genetically engineered land plant of claim 31, wherein the pyruvate carboxylase comprises a bacterial pyruvate carboxylase.

33. (canceled)

34. The genetically engineered land plant of claim 31, wherein the pyruvate carboxylase comprises an algal pyruvate carboxylase.

35. (canceled)

36. The genetically engineered land plant of claim 31, wherein the pyruvate carboxylase comprises a pyruvate carboxylase that is desensitized to feedback inhibition from aspartic acid.

37-43. (canceled)


SEQUENCE LISTING (80 sequences)

SEQ ID NO: 1 - protein, 336 aa, Chlamydomonas reinhardtii
MQTTMTRPCLAQPVLRSRVLRSPMRVVAASAPTAVTTVVTSNGNGNGHFQAATTPVPPTPAPVAVSAPVRAVSVLTPPQVYENAINVGAYKAGLTPLATFVQGIQAGAYIAFGAFLAISVGGNIPGVAAANPGLAKLLFALVFPVGLSMVTNCGAELFTGNTMMLTCALIEKKATWGQLLKNWSVSYFGNFVGSIAMVAAVVATGCLTTNTLPVQMATLKANLGFTEVLSRSILCNWLVCCAVWSASAATSLPGRILALWPCITAFVAIGLEHSVANMFVIPLGMMLGAEVTWSQFFFNNLIPVTLGNTIAGVLMMAIAYSISFGSLGKSAKPATA

SEQ ID NO: 2 - protein, 448 aa, Chlamydomonas reinhardtii
  misc_feature (222)..(222): Xaa can be any naturally occurring amino acid
  misc_feature (255)..(255): Xaa can be any naturally occurring amino acid
MFALSSRQTARSACRASCPCASCRGVASAPVRATYAARPVKKSAASVVVKAQAASTAVAPVENGAAPAVAHKRTFAQRHSELIKHFPSTMGVDDFMGRVEVALAGFGFTGDNTIAMTNLCRDEVTQVLKDKIEAIFGSSFNTNGLGGVLTCGVTGMKAGLSHSPVCNGGRERYVFFAFPHIAINSEGEMGALSRPGRPKQSCACGALLAILNAFKVDGVEKXCKVPGVHDPLDPELTILQQRLARRVRYEKLDVXKLDLPGLTSVAERTITDDLEYLIEKAVDPAVADYAVITGVQIHNWGKELSASGDASIEFVAPAKCYTVVNGLKTYIDLPQVPALSPRQIQTMAQASLNGFEPKHIQPGMRGSVISEVPLEYLVTKLGGSQLMEDGNSYAPVFASSDSFEWPTWQSRIRLDNNPNRLLSVERDANAPTMESPEPVHPSFEAPKN

SEQ ID NO: 3 - protein, 443 aa, Chlamydomonas reinhardtii
MALAQKMNVPVAAKAQGIVAPAVRPMAAARRVRSSIRAQASQALTVSQSKAVAPSNGAPAPLAQVEEVDIARHMNDRHAHILRYFPTALGVDDFMARTEIVLGGFGFTGDNTIAMTNLCRDEVTQVVKDKIEAAFGSSFNTNGLGAVLTCGVTGMKAGLSHSPVCAGGRERYVFFAFPHIAINSEGEVGAISRPGRPKMSCACGALQKCLVELKAEGVDAAVRAPGLHDPIEPEYSILKQRLARRIRYEKLDPQLMDLPSLTALAERTISDDLEYLIEKAVNPATSDYAVITGVEIHNWAAHLEEGGDPSMEFIAPTKAYVVVNGVKTHLDLMMVPPMSFRQLQLMAARSLADVPPGDICAGQRGSVLQEIPYGYLEKRMGGAATTGTVGRAANPVNLQIAAEWPSWQSRIRRDNNAAPYTLHQLERDMSAPTMDSPELANMN

SEQ ID NO: 4 - protein, 478 aa, Chlamydomonas reinhardtii
MPRTPFSRSVASQLASALEANLTQTSEPFAAPLWNAARPRMMSTIARSEGLLARSAAAPVGALKPCSCGKAVCAGHCSCGRAFCPGGHSNSLSTSTAAQNQPAWATDARAPGLAERLAEVTKHFPTSLSVDDFMARVEVALAGYGFTGDNSIAMSNLCRDESCLILEDKIEAAFGSCFSTHGLGGVLTCGVIGMKAGLSHSPVVGGKERYVFFSFPHIAIDSDGKVGAVSRPNRPGASAACGALIACMGDLKRDGLEANCKQPGVHDPLEPEYSILKQRIARRLAYEKINPLDCSLVDVTKAAERVISADLEYLISKAVDPKKADYAVFTGVQIHNWAADLNNTDVPSLEFVGVGKSYVVVNGEKVHLDLEKVPALSPRQLQILASASASEGKAATAASTGKLVQEIPREYLMRRLGGAMSRSHSDGAAPAWGSYVRKASLNDPHAGAPQMDHPFEATAAPKEDAGASTTSFFWGKKK

SEQ ID NO: 5 - protein, 441 aa, Chlamydomonas reinhardtii
MPRASFSRSVATQIASALEANLTPTFEPTAAQLWNAARPRMISTIARAEGSSLLRNVARGSGSSSVLKPCTCGKPAWATDARAPGLAERLAEQGVEVALAGYGFTSDNSIAMSNVRHDESCLILEDMIEAAFASCFSTHGLGGVLTCGVIGMKAGLSHSPVVGGKQCYGSFSFPHIAINSDGKVGAVSRPNRHGAGAACGALTACMGDLKRDGLEANCKQPGVHDPLEPEYSILKQRIARRLAYEKINPLDCSLVDVTKAAERVISADLEYLISKAVDPKKADYAVFTGVQIHNWVADLNNTDVPSLEFVGVGKSYVVVNGEKVHLDLEKVPALSPRQLQILASASASEGKAATAASTGKLMQEIPRKYMMRRLGAAMSRSHSDGAAPAGASLARGFQTCRHRCCVLLFLVDILQRAARVVAAKPTYTDGRQCRKREHGQD

SEQ ID NO: 6 - protein, 382 aa, Zea nicaraguensis
MCMGNHYHTSVGQQQAEAAMADDSPHAPSLTARHLEVAKHFPTAMGVDDFIARLEMALAAYGFTGDNAIAMSNLCRDESCMILEDKIESVFGSCFSTHGLGGVLTCGVIGMGAGLSHSPVENGKERYVFFSFPHIAIDSEGKVGAIARPNRPGASAACGALIKTMLDLKEEGVDAAVSSPGAHDPLEPEYSILKSRIARRIKYEKMDISNMSLVDVTKVAERVITTDLEYLISKAVNPKQADYAVVTGVQIHNWANDLEDERIPSMEFVAPARAYVVVNGEKIDLDLQQVPALSPRQLQLLAAQSTQVVENSRSLTTGTPNSMLQEIPRDYLLNRLGGVNTSIHLDVEHQGPSWREYIKTTFHDAHHNAPKMDEHFFEDKQQ

SEQ ID NO: 7 - protein, 434 aa, Cosmos bipinnatus
MQTAMKMNMQKATAPAAPRSSRMAAPVCAATCMCSACTGLRKVPTALSGQAPARMRSSAARRAVVAAAAPVLDKPTAQVSDQTNLQERHTCISQHFPSALGVDDFMARTEVALSGFGFTGENSIAMTNLCRDEVTTVLKDKIEAVFGSSFNTNGLGAVLTCGLTGMGAGFSHSPISNGKEHYVFFAFPHIGINSAGEVGAITRPGRPVKSCACGALQKCLIELKAEGYSKNCKVPGVHDPLDPEYSILKQRLARRVRYEGLDPTKMDLVSITKLAERTITNDIEYLSEKAVDIKKANYAVVTGVQIHNWATELDAKSGVPSLEFVAPAKVYVVVDGKKTFIDLSRVPTMSPRQLQLMAKASISGTRDEDVVAISKTVAGTLKEIPLKYLTQRLGVTKDPEELTMPGTSYEWTKAIVARDVTDSADDAEHTSFSQ

SEQ ID NO: 8 - protein, 405 aa, Nymphoides peltata
MAAVVPSAQTSFASSIAKGSPMKSSVLGNRIPLARTSRTVAASVPVKVFARSQHSSDSGANATFASVSSTAPPPSAAPNNAFVSGLVGGGIVAAAFLAFANTKKTSSNTATPAVPAPASKLPPVPRATQAAPAALETMSKFFPNAIQDERFVHLVAEELFKLGFTRDNCIAMVNTCRDEVCRPLVTTIDKEFGLSFNISGLGGLVNCGKTGLKAGMSHSPEFPCDVDGNPRERYVFFAFPHVSVGETGEVGSLLRRGRGKPSNACGALIAIKNTAAGGPNLPHDPLDDEFVLLKNKVLSQPICKNVSADGLSLVTVTKATLQTITDDLENLISKTVNPETSDYAVITGVQIHSGNQIPGEPFRIERTVDYVSAGTLYAVIRGQKHVFKAEDNEIKLVGSPVATGV

SEQ ID NO: 9 - protein, 358 aa, Chlamydomonas reinhardtii
MSSDAMTINESLMEVEHTPAVHKRILDILPGISGGVARVMIGQPFDTIKVRLQVLGQGTALAAKLPPSEVYKDSMDCIRKMIKSEGPLSFYKGTVAPLVGNMVLLGIHFPVFSAVRKQLEGDDHYSNFSHANVLLSGAAAGAAGSLISAPVELVRTKMQMQRRAALAGTVAAGAAASAGAEEFYKGSLDCFKQVMSKHGIKGLYRGFTSTILRDMQGYAWFFLGYEATVNHFLQNAGPGVHTKADLNYLQVMAAGVVAGFGLWGSMFPIDTIKSKLQADSFAKPQYSSTMDCLKKVLASEGQAGLWRGFSAAMYRAIPVNAGIFLAVEGTRQGIKWYEENVEHIYGGVIGPATPTAAQ

SEQ ID NO: 10 - protein, 513 aa, Ettlia oleoabundans
GHYAVLIDSLTTFINTLLLFDINKDSIMAALAQSTLQQARADCSAALNSARRLRRNTKAAQLFATRSTHKVDRKTVLRATAEATSSVIDAGGKTIIVESDGTIIIGSPEAVARTQAAKDTTENVPETVEVEYLTGRANAVQKQFEGALGADDFMQRVEMALYAFGFTGDNSIAMVNLCRDEVTVTLKHRIEEVFGSAFSTNGLGGVLTCGVTGMGAGFSHSPLCSSNKERYVFFSFPHISINASGEVGPMSRPGRPGQSCACGALIKATNEIKSEGLTCNCKIPGVHDALDPEMSILKQRIARRLRHEGFTDETVKGLSLVDVTKVAERTISDDLEFLISKTVNTDKADYAVVTGVQIHNWSNDFEDASPNMEFVAPTSAYVVVDGVKTHLDLSAMPPMTPRQMRLVAGPADVCSQGGQTMLREEEAPYAFDSKDSRRAQRARLQRYLSLMKEEGLDGTGATAVPSWQSKIVKGTPERCATADNSTIIDTSFAENAELRKVWEQLEEKYKMPN

SEQ ID NO: 11 - protein, 258 aa, Ettlia oleoabundans
KERYVFFSFPHIAIDSEGKIGAISRPNRPGASAACGALIKTMLDLKEEGVDKNVSSPGAHDPLEPEYSILKSRIARRIKYEKGDVQEMSLVDITKVAERVITTDLEYLISKAVNPKKADYAVVTGVQIHNWAADLEDGRVPSMEFVAPARAYVVVNGEKIDIDLQQVPALSPRQLQLMAAQSQQVADNTRSLTTGTPNSMLQEIPRDYLLNRLGGVNTSIHLDVEHQGPSWREYIKTTFHDAHHNAPKMDEHFFEDRQ

SEQ ID NO: 12 - protein, 499 aa, Ettlia oleoabundans
MPLALRAASLRTTCSCCSGGAHKASAPRASRSNLHAGRSTSRRTPHKAEARRQRVIFTNAAAADAAAANEETVIEAGGKMIIVESDGTIIIGGPEAVARAAAKADALEAAPESAEVEYLTGRANAVQKQFEGALGADDFMQRVEMALYAFGFTGDNSIAMVNLCRDEVTVTLKHRIEEVFGSAFSTNGLGGVLTCGVTGMGAGFSHSPLCSSNKERYVFFSFPHISINASGEVGPMSRPGRPGQSCACGALIKATNEIKSEGLTCNCKIPGVHDALDPEMSILKQRIARRLRHEGFTDETVKGLSLVDVTKVAERTISDDLEFLISKTVNTDKADYAVVTGVQIHNWSNDFEDASPNMEFVAPTSAYVVVDGVKTHLDLSAMPPMTPRQMRLVAGPADVCSQGGQTMLREEEAPYAFDSKDSRRAQRARLQRYLSLMKEEGLDGTGATAVPSWQSKIVKGTPERCATADNSTIIDTSFAENAELRKVWEQLEEKYKMPN

SEQ ID NO: 13 - protein, 6 aa, Chlamydomonas reinhardtii
FSFPHI

SEQ ID NO: 14 - protein, 5 aa, Chlamydomonas reinhardtii
ACGAL

SEQ ID NO: 15 - protein, 5 aa, Chlamydomonas reinhardtii
ADYAV

SEQ ID NO: 16 - protein, 8 aa, Chlamydomonas reinhardtii
TGVQIHNW

SEQ ID NO: 17 - DNA, 1500 bp, Zea mays
agttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 18 - DNA, 1500 bp, Zea mays
caacttacaagcgatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtaatttgcagttggaaatcattcttcgatcaatagggaaactctgagtcacagcgaaaggaaggttaattgcctacgttgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 19 - DNA, 1500 bp, Zea mays
tctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 20 - DNA, 1500 bp, Zea mays
cgagaatatatgttatcttcgtcgttagagaaatctagacagtatacaacaagatccacgtactacaggtaaacttttaggggtattgtgaacaagaggatgagtaaactctaaaagaacaaagctccaatgaaaatttaggtttttatgtggttagtcatagggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 21 - DNA, 1500 bp, Zea mays
cgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 22 - DNA, 1694 bp, Zea mays
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 23 - DNA, 1500 bp, Zea mays
tttaaatttggaacgtcgatccaacatctaacagaagcaccaattttacaaagaacccctttcaccttcctcacttggtgggacggttcttaatcaaattaactgcagccgctggtatacatgtacatgtgggcccgcctagcccggcacggcacaggcccacaaaaacacggtccacaaaagcacgacccacaaaagcacatatctaattatgggccgtgccgtgccagcacgtgtgcccagtcatcggcccacaattagttatgtgtgccaggccgacccaaatagcccaaaataccttaatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 24 - DNA, 1500 bp, Zea mays
caaaattttctattttttaaaaaatatgaattctagatttgggattgaacacatctaggctacaacgttgaattgatgaacaatagtgcttgttaataaattgctcacattcacattgtcgctcttacttcaaccatcatacatccatctacagtggtcacccatatttaatcctatggactaaagatgacagatgaacttctctcgttatatatatcactgtcctacatatatgagaaatgatatgtcctaaactcacctaaaaacaacaacatagtttaaatttaatcatagatgagcctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacg

SEQ ID NO: 25 - DNA, 1500 bp, Zea mays
acacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatct

SEQ ID NO: 26 - DNA, 1500 bp, Oryza sativa
gcagctgttttcgcggtacagggtgcaacaaaagcccatgacggcccacacctgcctctctccgctccaaacaccgaaacaagggggtgggtgcaatgggccggcgctcgaagaccgcgaactctttccaacagcccagcgcattagcccctcctcctactctctctaccttctttttaacatgcgactttctttctgtggacgacggcatcaacgacgggagcaggagcgggggctgaagcacggtgcgtgggctcctggagtggcgacggcctctccggcgagcttcctctggcgaactccctccgctcctcctatggcgaaatccaaacaagggtcagtttcgactccaaccttctcccaccaccacctcctgaccgtgccaccacccggccttgtcggcactgaaaggcgtcaacttgtcagcgcgggcctgctcggtcggtctcctcctcccctatttcgtttagctttgcccccgccaccaacaccggcccacggcccatggccgaccccgcggctttggcgccgccatcgctatctcgccgctgtccttttttcatgaccttcggtgccatccctctaaattcgatgcacctccctggctctatctccctttacctccgaaatcctaaccctacccataatctctagtgagtcttgtctttatttatggcctctttgaatcgcaggattgataaaacgtaggattttgataggaatgtaagtgtaaaacacatgattgtaaaatagaggaaaaacataggaatggccgtttgattgaaccgcagaaaaaacacaggaattagatgagagagatagactcaaagttactaagagattgaagcttttgctaaatttcctccaaaatctctataggattggccattccatagaaatttcaaaagatttaataggattcaatcctttgtttcaaaaaacttcatagaaaatttttctatagaattaaaatcctctaaaattcctatgttttttctccaattcaaaggggcccttaggttggaatttggaaagtgttcgcgagaaatcaagcggtcgcacgttagcgaattaggatttccggaaacaaaggaccgactccgcctatccatcgtcacgagcacagtgtagaacctcccagacctcaagagaccgttcaaaaagcgcgcgcccaagcggggcccaccaacgcgtccccaccgtgtcgcctcctgattggttgtcccctcttcctttcacgcgaaccggcaccctcccgacccttccagaacccccaatccgacggccaggatcgcccgcgcgcgaacgttctagacccccgccacctccgccacaaaacctctgcccctcccctctccccccgcttcgtctcgttcgagaaatcagaaagagagagaaattcccacgcagcagcaagcaatccaatccgagagcgcgcgtttgcgattattcgctttcgattccgcgaggtttttggagagggaggagaaggaggaggag

SEQ ID NO: 27 - DNA, 1500 bp, Oryza sativa
acagcatttattgtagtctggtcaagcgtgtcacgctgcatgcaacgcagtacagcgcgttcctttacccggtctgtgaccagtcacagaccggtcagatcacgggttaggtggcgactggcggtctgacgcacgccttgccccatcccgtcaagacgaaagcctctaggcactcgtctcaagccggagctagcgtgttatctcttagagatggcacgttagccctggttagatttataccaggcttcatcctaaccattacaggcaaggtgttacacgaagaagggcaaaacatgcacgttgttaaactgacgcgtgggggacaagaatgaccggtctgacactggtcgcatcagcaacgggcagccacgatcccgcgtcatctccgtctccgccgggagtggaggtaggtgtgggctgtcccatcagaagggctcccggatggaaaccgtaccgatctccgcccattaaagagaaaaagaacagtccagtttggaaagagaagggtgcatgtggtatccccttgaagtataaaaggaggaccttgcccatagagaagggggttgattctttccagattcagagcctagaacgagggagaggtgggctcacactttgtaacttgtccatacacaaatccacaaaaacacaggagtagggtattacgcttccgagcggcccgaacctgtatagatcgtccgtgtctcgcgtttcttgctggctgacgatccttccacatacagagagagagagagcttgggatctcaccctaagcccccggccgaaccggcaaaggggggcctgcgcggtctcccggtgaggagcctcgagctccgtcagacatgttcagtttcattatattatgaaatgtcacgtactgtttgttctagttagtgaattgtcatatggtaagaatatataaaaattaggttttctggactctatcttccaatgtatttttggatcctataacaaaatattttcataaatatattttttaagaatctaaacttttttgaaataaaagagcaacaaagaaaataaaaacgctctctcgtaagtaactcgtgaagatccatcgagagccactcgtttgaatcgtcgacacaaaagaacacttcattgattgcttttcgtcaattagccgcacagcacagtactctccaatctgctaaaccaaaaccaatctcatccatccatacccttcttgacaccaagtggcaactcctgattggacgcgccctatcctacatggcacccccaagattctctcgataggctacaggggccacaccgaccctccacgtcatcgtccacgtcaccctcatcccggcccatccagccaatcccagcccagcaaaaaatcttcccaagtggccaccagataagcctctccacgtattaatacgccaagtgttcgtcgccatgacacagcacgcacacacaccccaccagcagcagcagcagtagctgagcttgaagcagcagagcgaggtagac

SEQ ID NO: 28 - DNA, 1500 bp, Oryza sativa
tccacctctgttggttgcatcgacgtcgcttccctagctcccgtctctagtccggatcctattcctccttggagaccgaagctaccgcaaccattgctcggtggttagcgagcgtggagctgtcctccccactttcgcgtcctcgttcgccaccacagccatacttcgcatggtgatgtcttctccttcactcaccgctaaactcagtgcaaccgtttctaccctagccccggccgccgctctcatagaggtgaaagttcatttacatgtaggtcccacatgttttatgttttttatttttcttttactgattagcatgccacgtaaatcaaaacaacaatccatagtgttttaagtatttttatttaatacgtgagatggagtacaaaaacgagagatgcaaagtgaacttgctaaaacacattttctggttgattacagtcgcttgttgagccattggatcggtcataggattcgtgctagcatacttaattacgcgtaactagttgtgctttataggttacaggtcgctaattagcggtctactggagaactttgctactatttttttcttcactgcatgcactcgatcaagtatgagtatttgtaccgaccagcgaaacacatatgtaattaaagtataaatatgtaattagtatatattagtagtatatttagacagtagttacaccctacatacacaccacttacatatataattagtatgtaattttgtaacttacatatgtaattttagtacttacatatgtaattttgagacttacattgtaaatacactaaaattacatatgtaatttagtaacctacaatgtaaatacatgccgactaacttttgatgaaaaatatggtgttataaatatagctactcccgaactttattccttctctgtgagatatcagtggaaacgctcggtggaatcgggggagtatttgggagcacgcgccgacgcgcgcgtcgtgcgtgccgtcgtctttgtcgcggtggagcggagcgcgcccacttgcgcgcctgggccggaggcgggcgcgccgggggttcgggaatcccctggagccacacgtaaaggcgcgggcgggagggagggaggggccagctaggataaggcacgcgcggccgctgcgattggggcgcttgtgaacaccggggcgccacgtggagaggacgttacactccagccgccaaatttccactcccacacccgcgctcccctcccctctcttttccgtgatcgcacctcgcccacgcgccccccgccacacacaatctctgcagctctccagcttcgttggaactcgcgaatctctctccgatcccaggtaaagcagcgaacgacgtcacgcacgacgctgctcggtggatttcgttccttgctggggaaaaccatgcagagacgaaggtgaatgatctgcttttgtgtacttgcgtttaccaggtgaagcgcgagcttggagttggaggggagatcgatcagggccagg

SEQ ID NO: 29 - DNA, 1500 bp, Oryza sativa
ataattaattaattaatcaatcacttttcgtgctgtaaaaaatctcacccgatttgctgaaacgaactgagccgggcgactgtgatattctttcacgatttctgtttgtggcagtgggacattgctgtttattcgaaacaattttcaagtaaaaaaaaatactcaatggtaaggttgctagtaatagtttaacagtttgtttgcagctcagcaaatttcgtttcctcacagatgacacataactgaaagcactcaatgtaatgttgtgcttagctgctaaagcatgtcacgtcttagaaaacaactactccaccatggagaatttttcctcctacttactcctcacatacttaccatctccatataagttcccttgtcgtatcatatgtcttattcttcttgagcacagttattacagcagattttgtagaatagttatcgcatcaaaattttcctatgtcacctttgatcatgtgttatgtgtgcctcttgagtcttagggttaatgtggttgtaatgtgtttaaaaaactatatgaaagctcgtgtgttgctacgggagagagatacctcgaatgaatgtgagagatctccatttgagttgtgtaccttgagagagtgaaagatcacactatttatagacggttaataatggttactgaggtcgattcaccacatcgtcttaaacatttaatgagcatcctccacgtgaaaagtagagatgatagcgtgtaagagtggttcggccgatatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcataag

SEQ ID NO: 30 - DNA, 1945 bp, Oryza sativa
aaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattc

SEQ ID NO: 31 - DNA, 2315 bp, Oryza sativa
gttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtggga

SEQ ID NO: 32 - DNA, 1194 bp, Oryza sativa
ttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttct

SEQ ID NO: 33 - DNA, 1500 bp, Oryza sativa
gcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgttaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctct

SEQ ID NO: 34 - DNA, 1148 bp, Glycine max
gcaacagaagacccaaaactcaaaaaagttagtttcgggccaacatttcctcttgagggatgacacgtgacctgctactctggcccttatctggcatgtccatccttcttggcgcgacatttaattcgtcgtcagaaataactgaaggacaccttgcttgtttctcttttggccgccaccggtcttgtcatcgtcgaaggcgcccttgcgcttgtcggcagaacctttttcggcgacctccttgccttttcctttggccttgttcgtcatttctacagagaatgcaatgagaccaacgccaattgcatggttagagttagagaaatggagagaggaagaagtgcgtgactagagtgtgtgtaactgtgaagaacgacgagtccaaaatgaattttactgtaaataatttgaggaaaaaagtgatcaatacatatcatgcggtgcatacaagaatcggccattggtcaacttgtgagaggaaaaaatcatttaactaataccaaataatcttaaaattaataaaataatttaactaattaacccacggaagaaccttcttccgttgactctggcggaagaagttcttccgcatagttccatggaagatggttcttccgcagttcttctttcgttgacactcgcggaagaaatgttccacgggcgtccgcggaagaactttcttccgcaaagctaaagagcatttttgccatgtcgaaatcatcgccaatgaccagggtaacagaaccacgccctcttatgttggtttcaccgattcagagcgtttgatcggtgatgccgccaagaatcaggtcgccatgaaccccgtcaacaccgtcttcggtaagatccctagccgacacttcgccttttcaggatttgcattgttcctagatttttggatctgttgtttgaaactccacttttctattttggtaatttttagttttattttgtaatcctgctgtttatatgtcttattgttattattaatcgttgcatggtctgaactggtttagaactctacttgtattgtttgttaaaatcttatttgaaatcgaatagtaatataattttaatcgaatggtgatatgcataaacatcgtatttgttcgtcgaattctggttttgaattgaataatattgttatg

SEQ ID NO: 35 - DNA, 1378 bp, Glycine max
ctagaaattaaatgtttttaacaggtaatttgagaaaaatgtacttcaaaataattagttttaccagtttatgtcttctttttctcttttttatctttattctatgtttcaaattctaataatacatcatttaaatatttttaatttaaaagtgcttactaaattttaaaaaaatcatatttatcaaataacttctactttaaatttaaacttcattatttttaacttaaaaataacttttaaattaaaaaaatgaaaacaaacactacctaaaccctaaacactatctatctaagtcacattacttaatgattcttaatttatgttctttgtaaactttcatttcttcctccttttggctatacatgttcatttctgtgtactttactatattattagtaaaagccttttatataggtatatcaaatcaaataattaatataatatataattctcttaatttcatttcttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaag

SEQ ID NO: 36 - DNA, 1500 bp, Glycine max
gatcctcacaaacctcacttggagacataggtgtgagggtaacctttttccctttatgtacaaatgaaaatttgtttgtgacaccattatggacaacatccttacactactaaaaaagcttttttttacgacatcatatttacgacagtcatacaaaaacgtcttagtatgtataaggatggcaatttcgtaaatatttcaaacatttcaaaggcagtttcagaaaaccgtctttgaatgcggccattttaatttttaacgcgcccctcgcatccgttcctcttctttccgcaaatgtggtgctcgttccttttctttcccagctggcatctgttcctctccccactcgctagctatcttctgcttctcctcttctctcctcttcccattacatttctccaccttctccctggtaccaccaccgccccccactccacattcgtcctccgcccccattcccctatcctccagtaaaattacaaaaaaccctaacaccaaaaaaacccaaacccctgtcgcaatgaaatctccacccccaaatagctctttggaatagaatcaaggaacttaccaaatccattatatgctattggggttttggcatgtttccggtgtgaaagaaggaaaaagaaatgcgtatgcgatggtgatgtacgtaggtacgccgaaggactacgaattctacatagccatactcgtgcttctcaaatcgctggctacgctcgacgttgaaattgatcttgctgtgattgcttcccttgatgttcctcctcgatggattcgagctctgtaagtctcactccttcaccatcatttgccactttatttttatgtacttttactttattattatttgtaacctgtatttttatttggtttcggatatctgttgctttattattcaccctggaatttggttgattttattatttttgaaaaataaggaaagagatttatttgttagcttaattgttttaattggcgaatatgtttttcttttcccttttttgcacagagtgaagctttgttcttagggtaatggattcccttttttgtgatgctagtggatgatttgactgattagtgtttagtggaatgaagaaccagaactagtagtaggtagagggaatcacttttggttttggatgtaaacttagaaatgtgcagcactgcacagaattgatatttgatcgtgggtcaaattgtcaaaatgtgcaaagaatacaaaggcacaggtgatatcattccattttacgttttttaacgaagctgttagtttcaattcaattatttacatatataataaatatattgatacttgctttagtttcatgaattaaaagaatttgattttgtaaatttcatttgaatttgtttttgtacaagctctcaacttttattatatgaacgagaagtttcttttttcctttttgagtttatttgaacttgtggtgttctaattgtatatatttttgtgcaggtgtcaatcggtactactac

SEQ ID NO: 37 - DNA, 1261 bp, Glycine max
atctctcgacagttgcgaactgaacgctgagttggtaatgctatgccctatcgctttttgcaccgtcccatgatcatttcccccacaccaccccatcaacctctaaaaagttaagagtgaaaattacacacacccgaggagaagaaaagctgcttcttctaagcatcacaacctagttactttacttgtagggccttttccatttcccctaaattacccctcttttcatcatatgataataatatccagctcagactatagtatgatattatgatgtcagcataataggttggcactaaagtcttaaagggcattgtacatgttgcacctggcattcaaattcataaatactaacactgtgaaatagattataaatcctcaaataaatgtcacacggttggggttcgaatccactcaaaaaggctaatgggatgggatttaagtgccaaggaatataccatggactttaacagcaacacaatttacaatctaaaatgtattacttttttttttcaaaaaagatatacaaaataaggtaccaagaataaaaggagtatttagaaacagtggcaccaatttaataaattatttatataaaatgacacttatttaatttatcaatgataaaagtaatattgatttattctctgattaactgttcaattaatagtgttattatcataatctgtcgcaaaagttatttttatcaacaacaataattgatacaagtagtataaaattaagcctcttagttaatatagactacttgatactaaaaccatgttacaccaaaaagtaatttttatgtcacttgtctatataataattacgactaaattaataatttttaaaaatattactgaatccattaaccgaacttttataatgaaagtatttttatgctttaaaatcacaaacattgaataaactaaaaatgataccacggaattggaacaagagacgttccacacaaaagaaaaaaatatgttgaataattgaaacggtgacaagaaaagtggaataataatacaaagatggcagatggggttattgttattggaggagatgagtgaaataatgagtgaggggggtgtaactggaaagcaagaaaaagcgcaagagtgccagctatttccaacaacaaacgtggcccgtgggatgcgatattcgtaacgaacggcgaggatggaaggacgtgcaatttgcgcttcatttgaggcgaatttcatttggccagaccttccttttttaaaccacaggg

SEQ ID NO: 38 - DNA, 1094 bp, Glycine max
tgtgtcaatgttgtttctggtgaattgacataatgaattctacctgtacggagtagagaataactatttacccaacaagaatgattatctcattaatttttgaagtagacgcaataacgaatatattatacattcagaaaaatttcaccatattattctcaaatcacaacaataatttgttttttttttgcttgatataaaaccaatactctatactttttaaggttaatttaaacttaaagagtatttttaagatgcatgtactttaaggaataatagaaacatgacaacatcataaaagaatgaagaaactgaatcataacgtagtttgttacgccttccatttggtggttgatttggatacaatctagattggtttgctaaatggtttataagttatgtagacgtttttattactactattttagacaaatcaaatacacaccttcactttattctattcaaataacatgatttttcctaacattttttaaaaaaattactttttaaatataaactaattattttagaaatagttttataaaaatccacgccaaaaaaattaagttgtttttataaatataaacatcgggcttcaatcttaaatttataaatgtacgaaataatttgacagttaaatggaaattgctagcatggaagtgtttttatcatttatcaaactcaaccaaactgaacatcagaataattattagtgacaaattttgcagcatatgaagtggcttgcatagctccaaggctggcgatcatatgtcagattagagcaggctctctttggtactatgatacatttcaagcaaataacaaccgtaaaaattcacgccaaaatttttggaacgaatctatatattattattttatttcttttgatttcatgtacgtacagtgcccgtaattgacatgtctttgttccttaatgcctttcccacgtggaacaggcacctagaaacttggactaagtagggaattgagggccatggactatagtgccaaaccaacatcattttatatatatatatatatatatatatatatatgctattgttttctatagtttttggaaattaatacttatc

SEQ ID NO: 39 - DNA, 1449 bp, Glycine max
atttgtactaaaaaaaaatatgtagattaaattaaactccaattttaattggagaacaatacaaacaacacttaaaacctgtaattaatttttcttctttttaaaagtggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 40 - DNA, 1321 bp, Glycine max
aaaaacacaaaaaaaaattatacaaaaatgtttctcacaacatgagaagtaaaatccctcaaagaatttcacatcatcatatcagaatcaaaggaatcaaaatcataggtcaaaaatacaaaaacaccaagaacactcaatttattaactaatttgcatcatgacatcaattggtccatcaaacacaacaatcttgtaattataatcgtaacgaaagaattacaatgcaataaacatcccaaaataaacctcaatttaatcctctaaggatccctatacatgttcattctaaccccaattgtgataaattcatcccttacctctaagcaggctcacgtgtgtagtctggcagtgatagaggcatctctagtggttttctaatagtcctcaagcttgtttttcctctagttgttctgttaggattttcaagcgttagagagaagaagaagagattggagcctctatttcactgttaccgtacaagggatatttttctcaccataaacattattttgcaaatcccaacgaaggagatgtccgtacataagttcgaaacctggtgctcgaatttcacgacgattcaatggttaacaagtccaagattgtatttttactgtgacagatttgagtgtatacaagaaaaagagagctccatgcgaggaatatttctctcacagtagacattatttcataaatcccaatggtaaaaatatgcaaaaatgagtttcaaacctgcttttaaaatttcatgacgactcaacggttaacgtgtccgggattatattttcactggaacaagtttgagtgcatgcgggaaaagagagggttttgggagaggaaaaaaggaaaacaaatttaagaggaagagagagcgtaaaaatttatcgtaaatgtaaaaaatgacctaatatatctctatttataactagggtactctcaatctattatttactcatttttttattttattattttataaaaaagaattttattttacttcctatcaaattaataaataaaacattcttcttattttctaagatcacatatttattttatttaccttaaaatcatcattttaattaataaaattatttcttcttatttatttaattacaaaaatcttattatttttttaaaattttatttatttttaaataaaatattttttaatttattttataaaaaatgagatgttacattgaattataaaataaatagccaacaataaatagccgacttgcttttgcattgactaaggaagtcaagtcatcaataaatataatttccagttggcaatattctcaaagttggtctatat

SEQ ID NO: 41 - DNA, 514 bp, Glycine max
agatttgatcgatacttcattaaattgacattttattttaacacataatacattattaaaaatataaataaacatttacagcgaagttatataattaaaagcctggtctatgtaatggtaggaaatttgaaaatctaaaagcaaacaaaaattgttgtttatggtgctaagttgcacctggaaagatgcattgtttagctaaaacattcacgtcgagtacttggtttgggaaaaaaagccattcaagcttagctggtcctctctcctgtctctctctctctgtctgtctctctctgtctgtctctctctcaagcacatacacaaacaaagtaagggctataaataggagggatggaagtggaagaaagtctatagcgaagtttcatttctttggattagaaatttttcccaaagctgatcgagaagccagccaggccaggtctgtagttttctttttttctttttaatattaattcattattgtgttcttcatcatataatataattaagcctt

SEQ ID NO: 42 - DNA, 702 bp, Glycine max
cgcgccgtacgtaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgc

SEQ ID NO: 43 - DNA, 579 bp, Glycine max
acgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgc

SEQ ID NO: 44 - protein, 356 aa, Gonium pectorale
MVSMTMNDTLNQVEHTPVNPPHKKVLELLPGISGGVARVMIGQPFDTIKVRLQVLGAGTALAAKLPPSEVYKDSMDCVRKMIRTEGPLSFYKGTVAPLIGNMILLGIHFPTFSSVRKQLEGDDHYSNFSYTNTLIAGAAAGAAGSLVSTPVELVRTKMQMQRRAALAGSVAGSAASSGAEEFYKGSVDCFKQVLSKHGIKGLYRGFTSTVLRDMQGYAWFFLGYEATVNYFLQNAGPGVHSKADLNYLQVMAAGVVAGFGLWGSMFPIDTIKSKMQADSLAKPQYTTTMDCLRKVLKTEGQVGLWRGFSAAMYRAIPVNAGIFLAVEGSRQGIKWYEENVEHIYGGVVGAAPGAAS

SEQ ID NO: 45 - protein, 354 aa, Gonium pectorale
MSSMTVNDTLNEVEHTPKDPPHKRVLELLPGISGGVARVMIGQPFDTIKTRLQVLGAGTALAAKLPPSEVYKDSMDCVRKMVRSEGPLSFYKGTVAPLFGNMILLGIHFPVFSHVRKQLEGDDHYSNFSYTNALISGAAAGAAGSLVSTPVELVRTKMQMQRRAALAGSAGSAAASSGAEVFYKGSVDCFKQVLSKHGVKGLYRGVTSTVLRDMQGYAWFFLGYEATVNYFLQNAGPGVHSKADLNYLQVMAAGVVAGFGLWGSMFPIDTIKSKMQADSLVKPQYSTTYDCVRKVLKTEGNNGLWRGFSAAMYRAIPVNAGIFLAVEATRQGIKLYEENVEHIYGGVVGTTTAA

SEQ ID NO: 46 - protein, 339 aa, Volvox carteri
MNDTLNQVEHTPPVHKRILDILPGISGGVARVMIGQPFDTIKVRLQVLGQGTALAAQLPPSEVYKDSLDCVRKMVRNEGPLSFYKGTVAPLVGNMVLLGIHFPTFSYVRKQLEGDDHYTNFSYTNTLLSGAAAGAAGSLVSTPVELVRTKMQLQSAASSASDEFYKGSVDCFKQVLSKYGIKGLYRGFTATVLRDMQGYAWFFLGYESTVNYFLQKAGPGLHSKADLNYMQVMSAGVVAGFGLWGSMFPIDTVKSKLQADTLATPQYRSTYDCLSKVLKSEGQAGLWRGFSAAMYRAIPVNAGIFLAVEGTRQGIKWYEENVEHLYGGVVGPATPAATS

SEQ ID NO: 47 - protein, 339 aa, Volvox carteri; identical to SEQ ID NO: 46

SEQ ID NO: 48 - protein, 353 aa, Ettlia oleoabundans
MPATAQVMNDTLMEVEHTPPVHKRILDILPGVSGGVARIMVGQPFDTIKTRLQVLGKGTIGAAGMPPEMVYNSGMDCVRKMMKSEGPMSLYKGTVAPLLGNMVLLGIHFPTFTKTRAYLEAGDAPGSFSPWKILAAGAAAGAAGSVVSSPTELIRTKMQMVRKNNILAQIKGSAAGGLNPEENYKGNWDCAKKIFRNHGLRGMYSGYLSTLLRDMQGYAWFFFGYEATIHYLAGPGKTKADLDYSQVMLAGVMAGFGLWGSMFPIDTIKSKIQADSLSKPEFKGTLDCVRRSVQIEGYGGLWRGVTAALWRAIPVNAAIFLAVEGTRQLIADTEESIDAFVDQVSGKTSEAAL

SEQ ID NO: 49 - protein, 353 aa, Chlorella sorokiniana
MVARTINETLMEVEHTPPVHKRVLDVLPGVSGGVTRVLVGQPFDTIKTRLQVMGQGTALAKMLPPSDVYINSSDCLKKMVRNEGALSLYRGVVAPLLGNMVLLGIHFPTFSNTRKYLESVDATPAGEFPYWKVLAAGGAAGLAGSFISCPSEHIRTKMQLQRRAALAAQMGLKAQGLETYKGSWDCAVQILRNHGIKGLYRGMTSTVLRDIQGYAWFFLCYEATLHALAGPAHTRSELDYKHVLGAGVMAGFGLWGSMFPIDTIKSKMQGDSLSNPQYRNTLDCLRQSVAVEGFGGLFRGFGAAMYRAIPVNAGIFLAVEGTRQLLNKYEGYIDEKLGISVPASAATVPAPAQ

SEQ ID NO: 50 - protein, 303 aa, Chlorella variabilis
MRTGVAVDLASGTAAGAAQLLVGHPFDTIKVNMQVGSADTTAMGAARRIVGTHGPLGMYRGLAAPLATVAAFNAVLFSSWGATERMLSPDGGCCPLTVGQAMLAGGLAGVPVSLLATPTELLKCRLQAQGGARPPPGMVYSLADIRAGRALFNGPLDVLRHVVRHEGGWLGAYRGLGATLLREVPGNAAYFGVYEGCKYGLARWQCIPTSELGPASLMTAGGVGGAAFWIVTYPFDVVKSRLQTQNIHALDRYHGTWDCMTRLYSAQGWQALWRGFGPCMARSVPANAVAFLAFEQVRAALSH

SEQ ID NO: 51 - protein, 323 aa, Chlorella variabilis
MQEIQMPAVPAPPTLAAPQPASGFVRFAKDSFAGTVGGIAVTMVGHPFDTVKVRLQTQPSVNPIYNGAIDCVKKTLQWEGVPGLYKGVTSPLAGQMFFRATLFSAFGASKRWLGTNADGTTRDLTTADYYKAGFITGAAAAFTEAPIDFYKSQIQVQMVRAKADPTYKAPYTSVGECIKATVRYSGFKAPFQGLSATLLRNAPANAIYLGSFEVLKQQASKYYGCAPKDLSAPVVMAAGGTGGILYWLAIFPVDVIKSAMMTDSIDPAQRKYPTIPSTAKALWAEGGLSRFYRGFSPCIMRAAPANAVMLFTVDRVSHLLSDH

SEQ ID NO: 52 - protein, 323 aa, Chlorella variabilis
MTAGKSGLHPAADYVAGAIAGSANIALGFPADTVKVRLQNRLNPYNGAWHCATSMLRNEGARSLYRGMSPQLVGGAVETGVNYAVYQAMLGLTQGPRLALPEAAAVPLSAAAAGAVLSVVLSPAELVKCRLQLGGTERYHSYRGPVDCLRQTVQQEGLRGLMRGLSGTMAREIPGNAIYFSTYRLLRYWVSGGDPAATAAAASGATVAAASQPRSLLAFLVDSASAVVCGGLAGMVMWAAVLPLDVAKTRIQTAYPGSYQDVGVARQLHMVYREGGIQALYAGLSPTLARAFPANAAQWLAWELCMQQMQQWGGGGGRGGSST

SEQ ID NO: 53 - protein, 328 aa, Chondrus crispus
MPSTTPLVDATSPAAATPDASATAVPAPVSIAAAAGPVYPPYAHALAGAGGGLATVTLLHPLDTLRTRLQSVERRAVLARRGDAVRAFKEILVREGAPALYRGVVPAAFGSVLSWACYFHWFQRARTIVKPAITHETGSHLLAGTIAGLMTSFATNPIWVVKVRLQLQRTGKSVAPGFKPYSGFFDGLKSITREEGVRGLYRGIGPSVWLVSHGAVQFTMYERFKERLRQDADPQSGTTVFHSLIASTGSKLVASLATYPLQVARTRMQERFADGRRYGNFHTAFMYIFRTEGIRGLYRGLSANVIRVTPQAAVTFITYEQILKLCAN

SEQ ID NO: 54 - protein, 306 aa, Chlorella variabilis
MPHNETTPAALPFYKTFAASAAAACTGEVATIPMDTVKVRLQVQGASGAPAKYKGTLGTLAKVAREEGVASLYKGLVPGLHRQILLGGVRIATYDPIRDFYGRLMKEEAGHTSIPTKIAAALTAGTFGVLVGNPTDVLKVRMQAQGKLPAGTPSRYPSAMAAYGMIVRQEGVKALWTGTTPNIARNSVVNAAELATYDQIKQLLMASFGFHDNVYCHLSASLCAGFLAVAAGSPFDVIKSRAMALSATGGYQGVGHVVMQTMRNEGLLAFWSGFSANFLRLGSWNIAMFLTLEKLRHLMGAPSAKH

SEQ ID NO: 55 - protein, 233 aa, Chondrus crispus
VSREGAAGLYAGIQAPLPFVAVFNATLFAANSTMRKVVGKGRPDDDLSIAQIGLAGAGAGAAVSFVACPTELVKCRLQAQPGAFNGAIDCTRQVVANRGMGGLFTGMGATMVREMPGNALMFMTYNATMRALCSPGQATKDLSASQLMFAGGMAGLAFWMPCYPIDFAKTLIQTDSETNPRYRGLLDCMRKTVKAEGVGGLYKGIGPCLARAVPANAVTFLIYQWTLQLLGHS

SEQ ID NO: 56 - protein, 194 aa, Chondrus crispus
MGRPDDDLSIAQIGLAGAGAGPAVSFVACPTELIKCRLQAQPGAFNGAIDCTRQVVANRGMGGLFTGMGATMVREMPGNALMFMTYNATMRALCSPGQATKDLSASQLMFAGGMACLAFWMPCYPIDFAKTLIQTDSETNPRYRGLLDCMRKTVKAEGVGGLYKGIGPCLARAVPANAVTFLIDQCTLQLLGHS

SEQ ID NO: 57 - protein, 352 aa, Erigeron breviscapus
MPATPQLMNETLMEVEHTPAVHKRILDILPGVSGGVARIMVGQPFDTIKTRLQVLGKGTIGAAGMPPEMVYTSGMDCVRKMIKSEGPLSLYKGTIAPLLGNMVLLGIHFPTFHKTRAYLEREDAPGTHTPWKILAAGATAGAAGSIVSTPTELIRTKMQMVRKNNILQQIKGAGAGGLNPEENYKGNWDCAKKIFRNHGVRGLYSGYLSTLLRDMQGYAWFFFGYEATIHYLAGPGKTKADLDYTQVMLAGVIAGFGLWGSMFPIDTIKSKIQADSLSKPEFKGTLDCLKRSLAVEGQRGLWRGVTAALWRAIPVNAAIFLAVEGTRQLIADTEESVDKFVNNLTGKETAAV

SEQ ID NO: 58 - protein, 354 aa, Zea nicaraguensis
MPIATGQVMNDTLMEVEHTPPVHKRILDILPGVSGGVARIMVGQPFDTIKTRLQVLGAGTIGAQGMPADMVYNNGMDCVRKMIKSEGPGSLYKGTVAPLLGNMVLLGIHFPTFTKTRAYLEQGDAPGTFSPWKILAAGAAAGAAGSVVSTPTELIRTKMQMVRKNNLMAQMKGAAATLNPEENYKGNWDCAKKILRNHGLRGIYSGYVSTLLRDMQGYAWFFFGYEATIHMMCTEGKTKADLNFLQVMGAGVIAGFGLWGSMFPIDTIKSKIQADSLSKPEFKGTMDCLKRSLAVEGHAGLWRGVTAALWRAIPVNAAIFVAVEGTRQLIADTEESVDAFVNNLTGSGSTAAAV

SEQ ID NO: 59 - protein, 141 aa, Poa pratensis
YKGNWDCAKKILRNHGLRGIYSGYVSTLLRDMQGYAWFFFGYEATIHYLAGQHGKTKADLEYWQVMGAGVMAGFGLWGSMFPIDTIKSKIQADSLSKPEFKGTIDCLKRSLAVEGYAGMWRGVTAALWRAIPVNAAIFLAV

SEQ ID NO: 60 - protein, 354 aa, Cosmos bipinnatus
MPSATPQVINDTLMEVEHTPAVHKRILDILPGVSGGVARIMVGQPFDTIKTRLQVLGKGTIGAKGMPADMVYNNGMDCVRKMIKSEGAGSLYKGTVAPLLGNMVLLGIHFPTFTKTRAYLEQGDAPGTFSPAKILAAGAAAGAAGSVVSTPTELIRTKMQMVRKNNILAQMKGAAATLNPEENYKGNWDCAKKILRNHGLRGIYSGYVSTLLRDMQGYAWFFFGYEATIHMMCTDGKTKADLNFLQVMGAGVIAGFGLWGSMFPIDTIKSKIQADSLSKPEFKGTMDCLKRSLAVEGHAGLWRGVTAALWRAIPVNAAIFVAVEGTRQLIADTEESVDAFVNNLTGSSSTTAAV

SEQ ID NO: 61 - protein, 297 aa, Glycine max
MGDVAKDLTAGTVGGAAQLIVGHPFDTIKVKLQSQPTPLPGQLPKYSGAIDAVKQTVAAEGPRGLYKGMGAPLATVAAFNAVLFTVRGQMEALLRSHPGATLTINQQVVCGAGAGVAVSFLACPTELIKCRLQAQSVLAGTGTAAVAVKYGGPMDVARQVLRSEGGVKGLFKGLVPTMAREVPGNAAMFGVYEALKRLLAGGTDTSGLGRGSLMLAGGVAGAAFWLMVYPTDVVKSVIQVDDYKNPKFSGSIDAFRRISASEGIKGLYKGFGPAMARSVPANAACFLAYEMTRSALG

SEQ ID NO: 62 - protein, 296 aa, Zea mays
MGDVAKDLTAGTVGGAANLIVGHPFDTIKVKLQSQPTPAPGQLPKYAGAIDAVKQTVAAEGPRGLYKGMGAPLATVAAFNAVLFSVRGQMEAFLRSEPGVPLTVKQQVVAGAGAGIAVSFLACPTELIKCRLQAQSSLAEAATASGVALPKGPIDVAKHVVRDAGAKGLFKGLVPTMGREVPGNALMFGVYEATKQYLAGGPDTSGLGRGSQVLAGGLAGAAFWLSVYPTDVVKSVIQVDDYKKPKYSGSLDALRKIVAADGVKGLYKGFGPAMARSVPANAATFVAYEITRSALG

SEQ ID NO: 63 - protein, 296 aa, Oryza sativa
MGDVVKDLVAGTVGGAANLIVGHPFDTIKVKLQSQPTPAPGQFPKYAGAVDAVKQTIATEGPRGLYKGMGAPLATVAAFNALLFTVRGQMEALLRSEPGQPLTVNQQVVAGAGAGVAVSFLACPTELIKCRLQAQSALAEAAAASGVALPKGPIDVAKHVVREAGMKGLFKGLVPTMGREVPGNAVMFGVYEGTKQYLAGGQDTSNLGRGSLILSGGLAGAVFWLSVYPTDVVKSVIQVDDYKKPRYSGSVDAFKKILAADGVKGLYKGFGPAMARSVPANAATFLAYEITRSALG

SEQ ID NO: 64 - protein, 324 aa, Triticum aestivum
MEFWPEFLASSGGHEFVAGGVGGMAGVLAGHPLDTLRIRLQQPPRPVSPGITAARVTRPPSAVALLRGILRAEGPSALYRGMGAPLASVAFQNAMVFQVYAILSRSLDRRMSTSEPPSYTSVALAGVGTGALQTLILSPVELVKIRLQLEAAGRKRQGPVDMARDIMRREGLRGIYRGLTVTALRDAPSHGVYFWTYEYARERLHPGCRRTGQESLATMLVSGGLAGVASWVCCYPLDVVKSRLQAQTQTHPPSPRYRGVVDCFRKSVREEGLPVLWRGLGTAVARAFVVNGAIFSAYELALRFLVRNNGRQTLVMEEMKCHDH

SEQ ID NO: 65 - protein, 296 aa, Sorghum bicolor
MGDVARDLTAGTVGGVANLVVGHPFDTIKVKLQSQPTPAPGQLPKYAGAIDAVKQTIAAEGPRGLYKGMGAPLATVAAFNALLFSVRGQMEALLRSEPGVPLTVKQQVVAGAGAGIAVSFLACPTELIKCRLQAQSSLAEAAAASGVALPKGPIDVAKHVVRDAGAKGLFKGLVPTMGREVPGNAMMFGVYEATKQYLAGGPDTSNLGRGSQILAGGLAGAAFWLSVYPTDVVKSVIQVDDYKKPRYSGSLDALRKIVAADGVKGLYKGFGPAMARSVPANAATFVAYEITRSALG

SEQ ID NO: 66 - protein, 323 aa, Solanum tuberosum
MCDELSRCLIWCCLRSASISPISVFSQMDIMKDLTAGTVGGAAQLIVGHPFDTIKVKLQSQPTPLPGQPPKYAGAIDAVRKTVASEGPRGLYKGMGAPLATVAAFNALLFTVRGQTEALLRSEPGAPLTVKQQILCGAVAGTAASFLACPTELIKCRLQAHSALASVGSASVAIKYTGPMDVARHVLRSEGGVRGLFKGMCPTLAREVPGNAVMFGVYEALKQYFAGGMDTSGLGRGSLIVAGGLAGGSVWFAVYPTDVIKSVIQVDDYRSPKYSGSFDALKKILASEGVKGLYKGFGPAITRSIPANAACFLAYEMTRSSLG

SEQ ID NO: 67 - DNA, 11295 bp, Artificial Sequence
  Synthetic construct pYTEN1
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgaaaatgcctcgagcgtcattttcccgtagcgtagctactcaaattgcgtcagctctggaggctaaccttacaccgacttttgaacccactgcagcccagctgtggaacgcagcccgtcccaggatgatatcaactatagcgagagcggaggggtccagcctactgcgaaacgtagctcgtggaagcggcagtagttcagttcttaaaccttgcacctgtggaaaaccggcttgggctacggatgctcgtgctccagggttagcagagagattggcagaacagggggtggaggtggcgctagccgggtatgggtttacttcagacaatagtatagctatgtctaatgtaaggcacgacgagtcctgcttgatactggaagatatgatcgaagcggccttcgcatcatgcttctccactcatggtctgggaggggtgcttacgtgtggggtaataggcatgaaggctgggctcagtcactcccccgtagtgggcgggaagcaatgttacgggtctttctccttcccacacatagccatcaacagtgacggcaaagtgggcgcagtctcacgtccaaatcgacatggggcaggggctgcttgtggcgccttaactgcctgtatgggcgacttgaaacgagacggacttgaggcgaactgcaaacagcccggcgttcatgaccccctcgagcccgaatacagtatccttaagcaacgtatagctcgaaggctagcttacgaaaagataaatcccttagactgcagtcttgtagacgtgacgaaggcagccgagcgagttatctcagccgatcttgaatatctgatctccaaagctgtagaccccaagaaggcagattatgccgtttttacaggagtgcaaatacacaactgggtggcggatttgaataacaccgatgtgccttcccttgagtttgtaggcgtaggaaaatcatatgtagtggtcaatggagaaaaggtccatctcgatttagaaaaggttcccgcactatcaccaaggcagcttcagatattagcgtctgcctctgcctccgagggcaaagcagcaacggcggcgtccacaggcaaattaatgcaagaaatacctcgtaagtacatgatgcgaaggctaggtgccgctatgtcaaggtcccattctgatggtgcggcaccagcgggtgccagcctggcaagaggttttcagacatgtcgtcacagatgctgcgtccttctatttttggtagacattttacaaagagccgcccgagtagtagctgcaaagccaacttatacggacggaaggcagtgccgaaaaagagaacatggtcaggactgaggatccatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 68 - DNA, 11629 bp, Artificial Sequence
  Synthetic construct pYTEN2
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgcctcgagcgtcattttcccgtagcgtagctactcaaattgcgtcagctctggaggctaaccttacaccgacttttgaacccactgcagcccagctgtggaacgcagcccgtcccaggatgatatcaactatagcgagagcggaggggtccagcctactgcgaaacgtagctcgtggaagcggcagtagttcagttcttaaaccttgcacctgtggaaaaccggcttgggctacggatgctcgtgctccagggttagcagagagattggcagaacagggggtggaggtggcgctagccgggtatgggtttacttcagacaatagtatagctatgtctaatgtaaggcacgacgagtcctgcttgatactggaagatatgatcgaagcggccttcgcatcatgcttctccactcatggtctgggaggggtgcttacgtgtggggtaataggcatgaaggctgggctcagtcactcccccgtagtgggcgggaagcaatgttacgggtctttctccttcccacacatagccatcaacagtgacggcaaagtgggcgcagtctcacgtccaaatcgacatggggcaggggctgcttgtggcgccttaactgcctgtatgggcgacttgaaacgagacggacttgaggcgaactgcaaacagcccggcgttcatgaccccctcgagcccgaatacagtatccttaagcaacgtatagctcgaaggctagcttacgaaaagataaatcccttagactgcagtcttgtagacgtgacgaaggcagccgagcgagttatctcagccgatcttgaatatctgatctccaaagctgtagaccccaagaaggcagattatgccgtttttacaggagtgcaaatacacaactgggtggcggatttgaataacaccgatgtgccttcccttgagtttgtaggcgtaggaaaatcatatgtagtggtcaatggagaaaaggtccatctcgatttagaaaaggttcccgcactatcaccaaggcagcttcagatattagcgtctgcctctgcctccgagggcaaagcagcaacggcggcgtccacaggcaaattaatgcaagaaatacctcgtaagtacatgatgcgaaggctaggtgccgctatgtcaaggtcccattctgatggtgcggcaccagcgggtgccagcctggcaagaggttttcagacatgtcgtcacagatgctgcgtccttctatttttggtagacattttacaaagagccgcccgagtagtagctgcaaagccaacttatacggacggaaggcagtgccgaaaaagagaacatggtcaggactgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 69 - DNA, 14743 bp, Artificial Sequence
  Synthetic construct pYTEN3
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacggatccaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgaatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagacccgggatacctgcaggttaccatggcacaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacggaattcaaaatgcctcgagcgtcattttcccgtagcgtagctactcaaattgcgtcagctctggaggctaaccttacaccgacttttgaacccactgcagcccagctgtggaacgcagcccgtcccaggatgatatcaactatagcgagagcggaggggtccagcctactgcgaaacgtagctcgtggaagcggcagtagttcagttcttaaaccttgcacctgtggaaaaccggcttgggctacggatgctcgtgctccagggttagcagagagattggcagaacagggggtggaggtggcgctagccgggtatgggtttacttcagacaatagtatagctatgtctaatgtaaggcacgacgagtcctgcttgatactggaagatatgatcgaagcggccttcgcatcatgcttctccactcatggtctgggaggggtgcttacgtgtggggtaataggcatgaaggctgggctcagtcactcccccgtagtgggcgggaagcaatgttacgggtctttctccttcccacacatagccatcaacagtgacggcaaagtgggcgcagtctcacgtccaaatcgacatggggcaggggctgcttgtggcgccttaactgcctgtatgggcgacttgaaacgagacggacttgaggcgaactgcaaacagcccggcgttcatgaccccctcgagcccgaatacagtatccttaagcaacgtatagctcgaaggctagcttacgaaaagataaatcccttagactgcagtcttgtagacgtgacgaaggcagccgagcgagttatctcagccgatcttgaatatctgatctccaaagctgtagaccccaagaaggcagattatgccgtttttacaggagtgcaaatacacaactgggtggcggatttgaataacaccgatgtgccttcccttgagtttgtaggcgtaggaaaatcatatgtagtggtcaatggagaaaaggtccatctcgatttagaaaaggttcccgcactatcaccaaggcagcttcagatattagcgtctgcctctgcctccgagggcaaagcagcaacggcggcgtccacaggcaaattaatgcaagaaatacctcgtaagtacatgatgcgaaggctaggtgccgctatgtcaaggtcccattctgatggtgcggcaccagcgggtgccagcctggcaagaggttttcagacatgtcgtcacagatgctgcgtccttctatttttggtagacattttacaaagagccgcccgagtagtagctgcaaagccaacttatacggacggaaggcagtgccgaaaaagagaacatggtcaggactgaaattctctagagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggagctcttatacgtatactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 70 - DNA, 13889 bp, Artificial Sequence
  Synthetic construct pYTEN4
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcaccatggatacctgcaggaaaggatcctataagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatcttaacaattgataaaaatgcctcgagcgtcattttcccgtagcgtagctactcaaattgcgtcagctctggaggctaaccttacaccgacttttgaacccactgcagcccagctgtggaacgcagcccgtcccaggatgatatcaactatagcgagagcggaggggtccagcctactgcgaaacgtagctcgtggaagcggcagtagttcagttcttaaaccttgcacctgtggaaaaccggcttgggctacggatgctcgtgctccagggttagcagagagattggcagaacagggggtggaggtggcgctagccgggtatgggtttacttcagacaatagtatagctatgtctaatgtaaggcacgacgagtcctgcttgatactggaagatatgatcgaagcggccttcgcatcatgcttctccactcatggtctgggaggggtgcttacgtgtggggtaataggcatgaaggctgggctcagtcactcccccgtagtgggcgggaagcaatgttacgggtctttctccttcccacacatagccatcaacagtgacggcaaagtgggcgcagtctcacgtccaaatcgacatggggcaggggctgcttgtggcgccttaactgcctgtatgggcgacttgaaacgagacggacttgaggcgaactgcaaacagcccggcgttcatgaccccctcgagcccgaatacagtatccttaagcaacgtatagctcgaaggctagcttacgaaaagataaatcccttagactgcagtcttgtagacgtgacgaaggcagccgagcgagttatctcagccgatcttgaatatctgatctccaaagctgtagaccccaagaaggcagattatgccgtttttacaggagtgcaaatacacaactgggtggcggatttgaataacaccgatgtgccttcccttgagtttgtaggcgtaggaaaatcatatgtagtggtcaatggagaaaaggtccatctcgatttagaaaaggttcccgcactatcaccaaggcagcttcagatattagcgtctgcctctgcctccgagggcaaagcagcaacggcggcgtccacaggcaaattaatgcaagaaatacctcgtaagtacatgatgcgaaggctaggtgccgctatgtcaaggtcccattctgatggtgcggcaccagcgggtgccagcctggcaagaggttttcagacatgtcgtcacagatgctgcgtccttctatttttggtagacattttacaaagagccgcccgagtagtagctgcaaagccaacttatacggacggaaggcagtgccgaaaaagagaacatggtcaggactgatctagaaatgagctcgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagctcgcgaattcccgggactagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 71 - DNA, 3495 bp, Chlamydomonas reinhardtii
atgccccctttccattcacagcttgaccacaccggcgaggtgccgttcaagaagattctgtgcgctaaccgcggtgaaatcgccatccgcatcttccgcgcgggcactgagctgggcctgcgcacggtggctgtgtactcgccggcggaccggctgcagccgcaccgctacaaggcggacgaggcctactgcgtgggcaccgccgacatgcagccggtgagctgctacctggacatggacgccatcatcaagatcgccaaggaggcggaggtggacgccatccaccccggctacggcttcctgtccgagaacgccgcctttgcgcgcaagtgcgcggaggcgggcatcgtgttcatcggccccaagccggagaccatcgaggcgatgggcgacaagaccgccgcccgccgcgccgctgtggagtgcggcgtgtccattgtgcccggcaccaacaacccgctgtcgtcgcccgacgaggcgcgcgagttcgcggccaagtacggctaccccgttatcctgaaggcggccatgggcggcggcggccgcggcatgcgcgtggtgcgccacggagagggggggagagaaggagcggagaggagggaaggcagagggctggcacgcgtggcgcggggcaacgtccgggcggtcggggcagtaggagctgtgcggcagggacgcgggtgtgcggacggaaccagccaaggcgcccctcgcaagcagcggctcgtcagctgcggcccctcaactgcccatcatgccgtggtggaggtggcccccgcgcccaagctgcccaacagcacccgcaaggcgctgtacgacgacgcggtgaagctggcgcggcacgtgggctaccgcaacgccggcactgtggagttcatggtggacaaggacggcaagcactacttcctggaggtcaacccgcgcgtgcaggtggagcataccatcacggaggagatcacgggtgtggacatcgtgcagagccagatcaagatcgccgtggagggaagcagcaggcagattgggtctgcgcagttcggtgaacggcgccggggccgcgctgtggtctccagcaatccatggcgggggggggcgaggggcgaacagcctgatgccgcgggtgggggcgcctgtcggggtagtgagcagtggggtgatgtggccccggactgcgcggcggcgctggtaggagacagagtggatatggggagggggagggttttgagagcgaggagagtgagaacgaacgaggcggcggtgctgggcttgactaggaggcgctcaaaaatccgcggcatcaagaccaacatccccttcctggagaacgtgatgcgccaccccgacttcctgtccggcgaggccaccaccttcttcatcgagcagcaccagcgcgagctgttcaactttgagcgccacggctcgctgcgctcctccaagctgctcacctacctggcggacatggtggtgaacggccccgaccacccgggtgccatcggcgcgcccccctccaagttcgtgccctccccgctggccatcccggaccagctggtgggcaacctgagcggccccggctggcgcgacgtgctgcagcgcgaggggccggacggctgggccaaggcggtgcgcgcgcacaagggcgtgctcatcacagacaccaccatgcgtgacgcgcaccagtcgctgctggccacccgcatgcgcacgcacgacatgctgaaggccgcgcccgccaccgccgccatcctgagccaggccggctcgctggagatgtggggcggcgccacctttgacgtgtcgctgcgcttcctgcacgagtgcccctggcgccgcctggagcgcctgcgcgagctcatccccaacgtgccgttccaggtgcggggcgtggcaggagccccagtttgctcgcatgtgggtgatggcgttgggagggcggcgctcgtgcgtgtgcgtcaaccccacgggttgaaagtaaatccctcaatacatgaacccgtccatcaaccccttctgcccctgccccctcccccccgttgtcaagcatgggccacgctctcctcctcgctctattgcattgggtttggggagggggccaagactcactcgcagtaccagctggactactacctggacctggcggagaagctggtggagcacggctgccacgcactggccatcaaggacatggctggcctgctcaagccgcgtgccgccaccatcctggtgggggcgctgcgccagcgcttccccaacaccgtcattcacgtgcacacacacgactccgcgggcacgggtgtggccacccagctggccgccgcggccgccggcgccgacatcgtggactgctgcgtggacagcatgtcgggcctgaccagccagcccagcatgggcgccatcgtgaacgcgctgcacggcacgccgctggacaccggcatcaacccgcgccacctgctgccgctgttcaactactgggagtccactcgcgagctgtacgcgcccttcgagtccaacatgaaggctgtgagcagcgatgtgtacgtgcacgagatgccgggcggccagtacaccaacctcaagttccaggccatgagcctgggcctgggcgaggagtggtccaacatctgcaccgcatacgcctcggccaaccgcgccctgggagacatcgtcaaggtgacccccagctccaaggtggtgggcgacctggcccagttcatggtgcagaacgggctggacgagcacaccctggtggagcgcgccgagaacctgtccttccccagctcggtcgtggagttcatgcagggctacctgggccagccgtccttcggcttcccggagcctctgcgcagccgcgtgctcaagggcaagcacaccatcgagggccgccccggcgccagcctgggcgccatggacctggcgggcctggagtaccgcctcaaggagaagtacggcgcaggcgccatcagccagcgcgacgtgctgtccgcggcgctgtaccccaaggtgtttgacgagtacatgacgcacgtgctcaagtacagcgacctcatcgagaagctgcccacgcgcgccttcctgacgcctctggaggaggacgaggaggtggagttcgagatcgccaagggtgtggccgccaacatcaagtacaaggcggtgggcgagctgcagccgaacggcaagcgcgaggtgttctttgaggccaacggcgtgccgcgtgtggtggaggtgggcgacaagaaggcggagcaggtcatgggcaagaaggcggtgcgcgagaaggccgacctggcggtgctgggcagcgtgggcgcgcccatggccggaaccatcatcgaggtgtcggtgaagaccggcgccatggtgaagccgggtcagcagctggtggtgatgaacgccatgaagatggagacggccatctgcgcgccggtgtcgggcgtgatcacgcaggtggcggtggagaagaacgacgcgctggacgccggcgacctggtggtgtacatcgac

SEQ ID NO: 72 - protein, 1165 aa, Chlamydomonas reinhardtii
MPPFHSQLDHTGEVPFKKILCANRGEIAIRIFRAGTELGLRTVAVYSPADRLQPHRYKADEAYCVGTADMQPVSCYLDMDAIIKIAKEAEVDAIHPGYGFLSENAAFARKCAEAGIVFIGPKPETIEAMGDKTAARRAAVECGVSIVPGTNNPLSSPDEAREFAAKYGYPVILKAAMGGGGRGMRVVRHGEGGREGAERREGRGLARVARGNVRAVGAVGAVRQGRGCADGTSQGAPRKQRLVSCGPSTAHHAVVEVAPAPKLPNSTRKALYDDAVKLARHVGYRNAGTVEFMVDKDGKHYFLEVNPRVQVEHTITEEITGVDIVQSQIKIAVEGSSRQIGSAQFGERRRGRAVVSSNPWRGGARGEQPDAAGGGACRGSEQWGDVAPDCAAALVGDRVDMGRGRVLRARRVRTNEAAVLGLTRRRSKIRGIKTNIPFLENVMRHPDFLSGEATTFFIEQHQRELFNFERHGSLRSSKLLTYLADMVVNGPDHPGAIGAPPSKFVPSPLAIPDQLVGNLSGPGWRDVLQREGPDGWAKAVRAHKGVLITDTTMRDAHQSLLATRMRTHDMLKAAPATAAILSQAGSLEMWGGATFDVSLRFLHECPWRRLERLRELIPNVPFQVRGVAGAPVCSHVGDGVGRAALVRVRQPHGLKVNPSIHEPVHQPLLPLPPPPRCQAWATLSSSLYCIGFGEGAKTHSQYQLDYYLDLAEKLVEHGCHALAIKDMAGLLKPRAATILVGALRQRFPNTVIHVHTHDSAGTGVATQLAAAAAGADIVDCCVDSMSGLTSQPSMGAIVNALHGTPLDTGINPRHLLPLFNYWESTRELYAPFESNMKAVSSDVYVHEMPGGQYTNLKFQAMSLGLGEEWSNICTAYASANRALGDIVKVTPSSKVVGDLAQFMVQNGLDEHTLVERAENLSFPSSVVEFMQGYLGQPSFGFPEPLRSRVLKGKHTIEGRPGASLGAMDLAGLEYRLKEKYGAGAISQRDVLSAALYPKVFDEYMTHVLKYSDLIEKLPTRAFLTPLEEDEEVEFEIAKGVAANIKYKAVGELQPNGKREVFFEANGVPRVVEVGDKKAEQVMGKKAVREKADLAVLGSVGAPMAGTIIEVSVKTGAMVKPGQQLVVMNAMKMETAICAPVSGVITQVAVEKNDALDAGDLVVYID

SEQ ID NO: 73 - DNA, 3615 bp, Chlorella variabilis
atggcaatctccccagaatctgccacgcccttccgcaagatcatggctgccaaccgcggcgagattgccgtgcgcatcgcccgtgccggcatcgaactgggcctcacgacgctggccatctacagcgctgccgaccggctgcagccccaccgcttcaaggcggatgagtcgtaccaggtgggggctcccgagatgacacctgtgcagtgctacctggatgttcaagggatcgtggaggtggccaagaggcagggagtggacgtcgtgcacccggggtacggcttcctgtcggagaatgcggcctttgcacgcgagtgccagaggcagggcatcacgtttgtggggccgctgccagaaacgattgaggcgatgggcgacaagacggtggcgcggcgcctggcgcaggagtgcggcgtgcctgtggtgcccggcacggacgatgccctggccagcgcagaggaggcgaaggtgtttgcggcggcagcaggctacccggtgatcctcaaggcccgcagcggcggcggcggccgcggcatgcgcgtcgtgcgcgcggaggatgagatggaggacctctttgcccgcgcctcgaacgaggccaaggccgcctttggcgacggcggcatgttctgcgaaaagtatgttgaggatccgcggcacatcgaggtgcagatcctggcagacaaccacggaggcgtggtgcacctgtacgagcgggactgctccgtgcagcggcgccaccaaaaggtggtggagatggcgcccgcccccggcctggcggcggaagtcaaggagaagctgtacgaggcggcggtgaagctggccaggcacattggataccgcaatgccggcaccgttgagttcatggtggacaagcagggcgctttctacttcctggaggtcaacccgcgcatccaggtggagcacacggtgaccgaggagatcaccggggtggacctggtgcagagccagatcaagattgcaggaggcgcgaccctggcggagctgggcctgggcgaccaggccgcggtgccgccccccagcggcttcgccatccagtgccgcgtcacttccgaggaccccgagcgcaacttccagcctgattcggggcgcatcacggcgtaccgctcgcccggcgggcacggtatccgcctggacggcgccatggcggcgggaaactcggtatcccgccactacgattccctgctggtcaaggtcatctgcaagtcgcccaccttcattggcgcggtgcagaagatgcagcgctcgctgtacgagttctacatccgaggcatcaagaccaacatcgccttcctggagaacgtgctgcgccaccccgagttcctgggcggcgccgccaccacatccttcatcgagcgcaacccggagctgtttgagttcgacacctcgggctccagcgagatttcccacctgctggagtacctggctgagcaggtggtgaacggggcgcagcacccgggcgccgtgggccctccgccggccaaggtggcccctgccccgccgccgctgccccccggggcagacccccacatcgtgcccgcgggctggcgggactacctgctgacccacgggccggagaagtgggcgcaggcggtgcgggagcaccgccagacgcggggcgtgctgctcaccgacacaaccatgcgtgatgcccaccagagcctgctggccacacgcatgcggacggtagacatgctgcgtgcggcccccgccaccgcgcacatcctggcgcgggcgggcagcctggaggtgtggggcggggctacctttgacgtggcgctcaggttcctgcacgagtgcccctggaggcgcctggagcagctgcgggagaagatccccaacatccctttccagatgctgctgcgcggcgccaacgcggtgggctacaccagctacccagacaacgcggtgctggcgtttgtgcgggaggccaagctggcgggggtggacatcttccgcgtcttcgactccctcaacgacatagaccagctcaagtttggcatagactcggtgcgtgcggcggggggcgtggtggagggcacgctgtgctacacgggcgatgtgagcaacccgcgggcatccaagtacactctggaatactacatgggactggcagagaaaatggtggaccacggtatccacgtgctggccatcaaggacatggcgggcctgctgaagccgcgcgccgctaccatgctcatcggcgccctgcgccagaggttccctgacctccccatccacgtgcacacccacgacaccgccggcactgccgtggccacccagctggcggcggcggccgcgggcgcagacatcatcgactgctgcatcgactcggtcagcggcaccaccagccagccgtccatgggggcgatcgtgcactcgctggcgggctcagacctggacacaggcatcgaccccgactcgctgctgccgttgatcgactattgggaccagacgcgcctgctctacgcgcccttcgaatccaacctgcgcagttcctcctccgatgtgtaccgccacgagatgcctggcgggcagtacaccaacctcaagttccaggcagcttccctgggcctcgcctctgagtggggccgcgtcaagcacgcctacgccgccgccaaccgcgccctgggcgacatcgtcaaggtcacccccagctccaaggttgtcggcgacctggcccagttcatggtttccaacagcctggatgagcacagcctggtggcgcaggcagacgcgctgtccctgccatccagcgttgtggagtacctgcagggctacctgggccagcccgtgggcggcttccccgagccgctgcggtcgcgcgtgctcaaggacaagccgcgggtgcaggggcggccgggcgcctccatgcctcccatggatctcaaggccttggagcaggagctcaaggaccgccaccacgggtcgatgtgcggcggctcagtctgctcctgcatcagcatacgcgacgtgctgtctgcggccatgtaccccaaggtgtttgaggagtacaagaccttcaccgcgcgcttcagcgagcatatcgagaagctgcccacccgcgccttcctggcgccgctggacgtggatgaggaggtggatgtggagatggcgccgggcaacgtggtcagtatcaagctgaaagcggtgggggagctgcagcccaatggcacgcgggaggtgttcttcgaatgcgatggtgtgcctcgcgtggtggaaatcaaggatctgggcaaagacacggtggctgccgcccgccgcccggctcgcgacaaggccgacgtcggcgacgccggctcggtgccggctcccatggccggggaggtgatcgaggtgaaggccgcgccggggcactttgtgaccgcagggcaggccctggtggtgatgagcgccatgaagatggagacgtcggtggcggcgcccaccagcggtaccgtatcccacgtatacgtcatcaagggcgaccagtgcgagacgggtgacctgcttgtgctcatcaagcccggcacagaggcgccgcaaaacggcgacggcggcggcggcagcggcgccgaggcggctgcagccacgacggccgtcgctgcggcctcctga

SEQ ID NO: 74 - protein, 1204 aa, Chlorella variabilis
MAISPESATPFRKIMAANRGEIAVRIARAGIELGLTTLAIYSAADRLQPHRFKADESYQVGAPEMTPVQCYLDVQGIVEVAKRQGVDVVHPGYGFLSENAAFARECQRQGITFVGPLPETIEAMGDKTVARRLAQECGVPVVPGTDDALASAEEAKVFAAAAGYPVILKARSGGGGRGMRVVRAEDEMEDLFARASNEAKAAFGDGGMFCEKYVEDPRHIEVQILADNHGGVVHLYERDCSVQRRHQKVVEMAPAPGLAAEVKEKLYEAAVKLARHIGYRNAGTVEFMVDKQGAFYFLEVNPRIQVEHTVTEEITGVDLVQSQIKIAGGATLAELGLGDQAAVPPPSGFAIQCRVTSEDPERNFQPDSGRITAYRSPGGHGIRLDGAMAAGNSVSRHYDSLLVKVICKSPTFIGAVQKMQRSLYEFYIRGIKTNIAFLENVLRHPEFLGGAATTSFIERNPELFEFDTSGSSEISHLLEYLAEQVVNGAQHPGAVGPPPAKVAPAPPPLPPGADPHIVPAGWRDYLLTHGPEKWAQAVREHRQTRGVLLTDTTMRDAHQSLLATRMRTVDMLRAAPATAHILARAGSLEVWGGATFDVALRFLHECPWRRLEQLREKIPNIPFQMLLRGANAVGYTSYPDNAVLAFVREAKLAGVDIFRVFDSLNDIDQLKFGIDSVRAAGGVVEGTLCYTGDVSNPRASKYTLEYYMGLAEKMVDHGIHVLAIKDMAGLLKPRAATMLIGALRQRFPDLPIHVHTHDTAGTAVATQLAAAAAGADIIDCCIDSVSGTTSQPSMGAIVHSLAGSDLDTGIDPDSLLPLIDYWDQTRLLYAPFESNLRSSSSDVYRHEMPGGQYTNLKFQAASLGLASEWGRVKHAYAAANRALGDIVKVTPSSKVVGDLAQFMVSNSLDEHSLVAQADALSLPSSVVEYLQGYLGQPVGGFPEPLRSRVLKDKPRVQGRPGASMPPMDLKALEQELKDRHHGSMCGGSVCSCISIRDVLSAAMYPKVFEEYKTFTARFSEHIEKLPTRAFLAPLDVDEEVDVEMAPGNVVSIKLKAVGELQPNGTREVFFECDGVPRVVEIKDLGKDTVAAARRPARDKADVGDAGSVPAPMAGEVIEVKAAPGHFVTAGQALVVMSAMKMETSVAAPTSGTVSHVYVIKGDQCETGDLLVLIKPGTEAPQNGDGGGGSGAEAAAATTAVAAAS

SEQ ID NO: 75 - DNA, 4704 bp, Chlorella sorokiniana
atgagcttggcggcggccctgcggcccagcagaccgccccaggctgtcggtaggcgggcatgccagatgccgcaggcgctgcaggcgccgccacggccggcgccgcggcggagaggctgggcggcgcttgcgcctgctgggcgccctgctgctccgctgcgtgttgcacggccgatggggcccctggatcccgagcagcatgaggagcaggagcagcaggctgccatccgcgagcggctgcagggctactggcggctggtgcgcggctggaatgcactgccttcaatggcgcttgtactgctgggtgcatggacgggcgccggcaagacgctgcttgccctcaagcacctcacagtttggttcatgggcctggcctcgggcgccgtggcaatggccagctgtacaatcaatgactatttcgatgctgacattgacgcagtgaacgacccgcagaagccggtgccgtcgggcctcattcctcgcgaccgtgcgctgctcgtggctgccctgctctacatcggtctgctggctctggcgtgcctggttcccaatgcaggcgtgcggctcatagtggcactttcttcggctctcactgtgctgtacactcccgtgctgaagaagcagacgttggtcaagaactgcgttgtggcgtgcgtcattgcggcagcgccgctggcgggcgccctcgcggctggcgcgggcggcgggccgggcctgcgggcggtgctggcgccctgtgccttcctgtggctgggcatcatgtttcgcgaaataatgatggatatccaggaccggtgcggcgacgggctagctggcgtgctcacgctgcctgttgtgctgggcccgcgtgccgccttgggcatcggcttcggcttgctggccgcctgcatggccctggcagcacacgccgcagtgtacggcagcggcctggcgtgggcctgggcggcagcacccactctggagcccgcagcgcgctcagcggcgctggctgccgtggcatgggtgctgtccacgccttgcggcgctgcactggcggtgcagcgcacccataatggaaatggagccaagcagttccagcccgttcgcgtgaacgagcggggcatcgtggtggacgatgggcagactatccccttcaagaagctgatggcggccaaccgcggcgagatcgcagtgcgcatcacccgcgcaggcattgagctggggctcaccacgctggccatttacagcgaggcggaccggctgcagccgcaccgcttcaaggccgacgagtcgtacgaggtgggcagctccgagatgacgcctgtgcaggcgtacctggacgtgcccggcattgtgcggctggccaaggagcagggcgtggacgtcatccaccccggctacggcttcctgtcggagaacgcggcatttgcgcgcgagtgccagaaggcgggcatcacttttgtgggccccctgcctgagaccattgaggccatgggcgacaaaaccgcagcccgccgcctggctgtggagtgcggcgtgccggtggtgcccggcaccaatgatgccctggagtcagcggagcaggccaaggcgtttgcgcgggaggcgggataccccgtcatcctcaaggcgcgctccggcggcggcggccgcggcatgcgcgtcgtgcacagcgaggaggagatggaggacaactttgtgcgggcgtccaacgaggccaaggccgcctttggcgacggcggcatgttcatcgagaagtacctggaggacccgcgccacatcgagatccagatcctggctgacaaccacggcaacgtggtgcacctctacgagcgcgactgctccgtgcagcgccgccaccaaaaggtggtggagatggcgcctgcccccggccttgacgaggggctgcgccaggcgctgtttgacgacgccgtcaagctggccaagcacgcgctttttgacgacgccgtcaagctggccaagcacgtgggctacaggaacgcgggaactgtggagttcattgtggacaagcacggcaagcactactacatggaaaccaacccgcgcatccaggtggagcacacggtgactgaggagatcacaggcatcgacctggtgcagtcccagatccgcatcgcgggcggcgccaccctggcgcagctgggcctgggcagccaggcggacgtgcccaagcccaacggctatgccatccagtgccgtgtgacgagcgaggaccctgagcgcaacttccagcccgactctggccgcatcaccgcctaccgctcccccggcgggcacggcatccgcctggacggcgccatggcggccggcaacattgtgtcccgccactacgactcgctgctggtcaaggtgatctgcaaggcacccaccttcatgtcggcggtgcagaagatgcagcgcgcgctctacgagttccacatccgcggcatcaagaccaacatcctgttcctggagaacgtgctgcgccaccccgagttcctgagcggcgaggccaccacctccttcatcgaccgcaaccccgagctgttccagctcaaccagaaggagctgtctgagctgtgccgcctgctggagtacctggcggagcagaaggtcaacgggcccaagcaccctggtgccattggcgcgccacccgccaaggtggcgcctgcccccgtgccgctgccgcacggctctgacccacacatcgtgcctgcgggctggaaggactacctcgacaagcagggccctgaggcctgggccaaggctgtgcgggagcaccgtcagagccggggtgtgctgatcacggataccaccatgcgggacgcccaccagtcgctgctggccacccgcatgcgcacgcacgacatgctcaaggcggcccccgccaccgcccacatcctggccaacgcgggctcgttggaggtgtggggcggcgccacctttgacgtcgcactgcgcttcctgcatgagtgcccctggaggcgcctggagctgctgcgcgagcggatccccaacgtccccttccagatgctgctgcgcggcgccaacgcggtggggtacacctcctaccccgacaacgcctgctttgcctttgtggacgaggccaagaaggcgggcgtggacatcttccgcgtctttgactccctcaacgacattgaccagctgcgcttcggcattgacacggtggcgcgagcgggcggcgtaattgagggcacgctgtgctacacgggcgatgtgtccaacccccgcgcatccaagtataccttggagtactacctcaacctggcagagaagatggtggagcacggcattcacgcgctggccatcaaggacatggcgggcctgctcaagccgcgggcagccaccatgctggtgggggcgctgcgggagcgcttccctgacctgcccatccacgtgcacacgcacgacacagcgggcacaggcgtggccacgcagctggcggcggcggcggcgggtgccgacatcattgactgcgccattgacagcatgagcggcaccacctcccagccgtccatgggcgccattgtcaactcgctggccggcacggacctggacacgggcatcgaccccgaggccatccagccgctgatcgactactgggaccaggcgcgcctgctctacgcacccttcgagtccaacctgtactgctcatcctcggatgtgtatcgccatgagatgccgggcgggcagtacaccaaccttaagttccaggccaccaccctgggcctgggcagcgagtgggagcgcgtcaagacagcgtatgcagctgccaaccgcgcgctgggagacattgtcaaggtcaccccctcctccaaggtggtgggcgacctggctcagttcatggtgtccaacaacctggacgagcactcgctggtggagcaggcagagacgctctccctgcccagcagtgtggtggagttcctgcagggatacctgggcacccccgtgggcggcttcccggagcccctgcgctcccgcgtcctcaaggacaagcccatagttcaggggcggccgggcgcgagcatggcgccgctggatatccgcggcctcgagtcgcagctgaaggagaagcacccagccatctcctaccgcgacgtcatgtccgccgccatgtaccccaaggtctttgaggagtacaagaccttcacggagcggttcagccggcatgtggagaagctgcccacgcgcgccttcctggcgccgctggacattgacgaggagattgacgtggaactgaccaagggcaacaaggtcagcatcaagctcaaggccatcggggagctgcagccctcgggcatgcgcgaggtgttctttgagtacaacggcatcccgcgcgtggtggaggtgcgagaggagtccaaggcggcatccgacaccaagaaggctgcgcgtgacaaggcggacagcagcgaccccggctccgtgggtgcgcccatggccggcgagatcatcgaggtcaaggccaagccgggatcgtatgtgaaggctggccaggcgctggtggtcatgtcagccatgaagatggagacgactgtggcggcccccgcctccggcactgtgtcccacgtggcggtcatcaagggagaccagtgcgacaccggcgacctgctggtcctgatcaagccaggagagcccaacggcagcggcagcaacggcagcggcacggcagacgccaagcccctggcgggcgcctcatcctga

SEQ ID NO: 76 - protein, 1533 aa, Chlorella sorokiniana
MPQALQAPPRPAPRRRGWAALAPAGRPAAPLRVARPMGPLDPEQHEEQEQQAAIRERLQGYWRLVRGWNALPSMALVLLGAWTGAGKTLLALKHLTVWFMGLASGAVAMASCTINDYFDADIDAVNDPQKPVPSGLIPRDRALLVAALLYIGLLALACLVPNAGVRLIVALSSALTVLYTPVLKKQTLVKNCVVACVIAAAPLAGALAAGAGGGPGLRAVLAPCAFLWLGIMFREIMMDIQDRCGDGLAGVLTLPVVLGPRAALGIGFGLLAACMALAAHAAVYGSGLAWAWAAAPTLEPAARSAALAAVAWVLSTPCGAALAVQRTHNGNGAKQFQPVRVNERGIVVDDGQTIPFKKLMAANRGEIAVRITRAGIELGLTTLAIYSEADRLQPHRFKADESYEVGSSEMTPVQAYLDVPGIVRLAKEQGVDVIHPGYGFLSENAAFARECQKAGITFVGPLPETIEAMGDKTAARRLAVECGVPVVPGTNDALESAEQAKAFAREAGYPVILKARSGGGGRGMRVVHSEEEMEDNFVRASNEAKAAFGDGGMFIEKYLEDPRHIEIQILADNHGNVVHLYERDCSVQRRHQKVVEMAPAPGLDEGLRQALFDDAVKLAKHVGYRNAGTVEFIVDKHGKHYYMETNPRIQVEHTVTEEITGIDLVQSQIRIAGGATLAQLGLGSQADVPKPNGYAIQCRVTSEDPERNFQPDSGRITAYRSPGGHGIRLDGAMAAGNIVSRHYDSLLVKVICKAPTFMSAVQKMQRALYEFHIRGIKTNILFLENVLRHPEFLSGEATTSFIDRNPELFQLNQKELSELCRLLEYLAEQKVNGPKHPGAIGAPPAKVAPAPVPLPHGSDPHIVPAGWKDYLDKQGPEAWAKAVREHRQSRGVLITDTTMRDAHQSLLATRMRTHDMLKAAPATAHILANAGSLEVWGGATFDVALRFLHECPWRRLELLRERIPNVPFQMLLRGANAVGYTSYPDNACFAFVDEAKKAGVDIFRVFDSLNDIDQLRFGIDTVARAGGVIEGTLCYTGDVSNPRASKYTLEYYLNLAEKMVEHGIHALAIKDMAGLLKPRAATMLVGALRERFPDLPIHVHTHDTAGTGVATQLAAAAAGADIIDCAIDSMSGTTSQPSMGAIVNSLAGTDLDTGIDPEAIQPLIDYWDQARLLYAPFESNLYCSSSDVYRHEMPGGQYTNLKFQATTLGLGSEWERVKTAYAAANRALGDIVKVTPSSKVVGDLAQFMVSNNLDEHSLVEQAETLSLPSSVVEFLQGYLGTPVGGFPEPLRSRVLKDKPIVQGRPGASMAPLDIRGLESQLKEKHPAISYRDVMSAAMYPKVFEEYKTFTERFSRHVEKLPTRAFLAPLDIDEEIDVELTKGNKVSIKLKAIGELQPSGMREVFFEYNGIPRVVEVREESKAASDTKKAARDKADSSDPGSVGAPMAGEIIEVKAKPGSYVKAGQALVVMSAMKMETTVAAPASGTVSHVAVIKGDQCDTGDLLVLIKPGEPNGSGSNGSGTADAKPLAGASS

SEQ ID NO: 77 - protein, 1545 aa, Chlorella sorokiniana
MPQALQAPPRPAPRRRGWAALAPAGRPAAPLRVARPMGPLDPEQHEEQEQQAAIRERLQGYWRLVRGWNALPSMALVLLGAWTGAGKTLLALKHLTVWFMGLASGAVAMASCTINDYFDADIDAVNDPQKPVPSGLIPRDRALLVAALLYIGLLALACLVPNAGVRLIVALSSALTVLYTPVLKKQTLVKNCVVACVIAAAPLAGALAAGAGGGPGLRAVLAPCAFLWLGIMFREIMMDIQDRCGDGLAGVLTLPVVLGPRAALGIGFGLLAACMALAAHAAVYGSGLAWAWAAAPTLEPAARSAALAAVAWVLSTPCGAALAVQRTHNGNGAKQFQPVRVNERGIVVDDGQTIPFKKLMAANRGEIAVRITRAGIELGLTTLAIYSEADRLQPHRFKADESYEVGSSEMTPVQAYLDVPGIVRLAKEQGVDVIHPGYGFLSENAAFARECQKAGITFVGPLPETIEAMGDKTAARRLAVECGVPVVPGTNDALESAEQAKAFAREAGYPVILKARSGGGGRGMRVVHSEEEMEDNFVRASNEAKAAFGDGGMFIEKYLEDPRHIEIQILADNHGNVVHLYERDCSVQRRHQKVVEMAPAPGLDEGLRQALFDDAVKLAKHALFDDAVKLAKHVGYRNAGTVEFIVDKHGKHYYMETNPRIQVEHTVTEEITGIDLVQSQIRIAGGATLAQLGLGSQADVPKPNGYAIQCRVTSEDPERNFQPDSGRITAYRSPGGHGIRLDGAMAAGNIVSRHYDSLLVKVICKAPTFMSAVQKMQRALYEFHIRGIKTNILFLENVLRHPEFLSGEATTSFIDRNPELFQLNQKELSELCRLLEYLAEQKVNGPKHPGAIGAPPAKVAPAPVPLPHGSDPHIVPAGWKDYLDKQGPEAWAKAVREHRQSRGVLITDTTMRDAHQSLLATRMRTHDMLKAAPATAHILANAGSLEVWGGATFDVALRFLHECPWRRLELLRERIPNVPFQMLLRGANAVGYTSYPDNACFAFVDEAKKAGVDIFRVFDSLNDIDQLRFGIDTVARAGGVIEGTLCYTGDVSNPRASKYTLEYYLNLAEKMVEHGIHALAIKDMAGLLKPRAATMLVGALRERFPDLPIHVHTHDTAGTGVATQLAAAAAGADIIDCAIDSMSGTTSQPSMGAIVNSLAGTDLDTGIDPEAIQPLIDYWDQARLLYAPFESNLYCSSSDVYRHEMPGGQYTNLKFQATTLGLGSEWERVKTAYAAANRALGDIVKVTPSSKVVGDLAQFMVSNNLDEHSLVEQAETLSLPSSVVEFLQGYLGTPVGGFPEPLRSRVLKDKPIVQGRPGASMAPLDIRGLESQLKEKHPAISYRDVMSAAMYPKVFEEYKTFTERFSRHVEKLPTRAFLAPLDIDEEIDVELTKGNKVSIKLKAIGELQPSGMREVFFEYNGIPRVVEVREESKAASDTKKAARDKADSSDPGSVGAPMAGEIIEVKAKPGSYVKAGQALVVMSAMKMETTVAAPASGTVSHVAVIKGDQCDTGDLLVLIKPGEPNGSGSNGSGTADAKPLAGASS

SEQ ID NO: 78 - protein, 1140 aa, Corynebacterium glutamicum
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 79 - protein, 1157 aa, Corynebacterium glutamicum
MTAITLGGLLLKGIITLVSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDDIVKSAEGQTYPIFVKAVAGGGGRGMRFVSSPDELRKLATEASREAEAAFGDGSVYVERAVINPQHIEVQILGDRTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIGDHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIERVVVPAATKVEGGDLIVVVS

SEQ ID NO: 80 - protein, 1148 aa, Bacillus subtilis
MSQQSIQKVLVANRGEIAIRIFRACTELNIRTVAVYSKEDSGSYHRYKADEAYLVGEGKKPIDAYLDIEGIIDIAKRNKVDAIHPGYGFLSENIHFARRCEEEGIVFIGPKSEHLDMFGDKVKAREQAEKAGIPVIPGSDGPAETLEAVEQFGQANGYPIIIKASLGGGGRGMRIVRSESEVKEAYERAKSEAKAAFGNDEVYVEKLIENPKHIEVQVIGDKQGNVVHLFERDCSVQRRHQKVIEVAPSVSLSPELRDQICEAAVALAKNVNYINAGTVEFLVANNEFYFIEVNPRVQVEHTITEMITGVDIVQTQILVAQGHSLHSKKVNIPEQKDIFTIGYAIQSRVTTEDPQNDFMPDTGKIMAYRSGGGFGVRLDTGNSFQGAVITPYYDSLLVKLSTWALTFEQAAAKMVRNLQEFRIRGIKTNIPFLENVAKHEKFLTGQYDTSFIDTTPELFNFPKQKDRGTKMLTYIGNVTVNGFPGIGKKEKPAFDKPLGVKVDVDQQPARGTKQILDEKGAEGLANWVKEQKSVLLTDTTFRDAHQSLLATRIRSHDLKKIANPTAALWPELFSMEMWGGATFDVAYRFLKEDPWKRLEDLRKEVPNTLFQMLLRSSNAVGYTNYPDNVIKEFVKQSAQSGIDVFRIFDSLNWVKGMTLAIDAVRDTGKVAEAAICYTGDILDKNRTKYDLAYYTSMAKELEAAGAHILGIKDMAGLLKPQAAYELVSALKETIDIPVHLHTHDTSGNGIYMYAKAVEAGVDIIDVAVSSMAGLTSQPSASGFYHAMEGNDRRPEMNVQGVELLSQYWESVRKYYSEFESGMKSPHTEIYEHEMPGGQYSNLQQQAKGVGLGDRWNEVKEMYRRVNDMFGDIVKVTPSSKVVGDMALYMVQNNLTEKDVYEKGESLDFPDSVVELFKGNIGQPHGGFPEKLQKLILKGQEPITVRPGELLEPVSFEAIKQEFKEQHNLEISDQDAVAYALYPKVFTDYVKTTESYGDISVLDTPTFFYGMTLGEEIEVEIERGKTLIVKLISIGEPQPDATRVVYFELNGQPREVVIKDESIKSSVQERLKADRTNPSHIAASMPGTVIKVLAEAGTKVNKGDHLMINEAMKMETTVQAPFSGTIKQVHVKNGEPIQTGDLLLEIEKA